SEQUENCE LISTING (41 sequences)

SEQ ID NO: 1 - protein, 11 aa, Artificial Sequence
  Description of Artificial Sequence Synthetic peptide
  CDR-L1 Antibody 1
QSLLNSDGNTY

SEQ ID NO: 2 - protein, 3 aa, Artificial Sequence
  Description of Artificial Sequence Synthetic peptide
  CDR-L2 Antibody 1
LVS

SEQ ID NO: 3 - protein, 7 aa, Artificial Sequence
  Description of Artificial Sequence Synthetic peptide
  CDR-L3 Antibody 1
VQGTHDP

SEQ ID NO: 4 - protein, 8 aa, Artificial Sequence
  Description of Artificial Sequence Synthetic peptide
  CDR-H1 Antibody 1
YHTITSGY

SEQ ID NO: 5 - protein, 7 aa, Artificial Sequence
  Description of Artificial Sequence Synthetic peptide
  CDR-H2 Antibody 1
ISYSGNT

SEQ ID NO: 6 - protein, 13 aa, Artificial Sequence
  Description of Artificial Sequence Synthetic peptide
  CDR-H3 Antibody 1
ASMMVPHYYVMDA

SEQ ID NO: 7 - protein, 16 aa, Artificial Sequence
  Description of Artificial Sequence Synthetic peptide
  CDR-L1 (Kabat) Antibody 1
RSSQSLLNSDGNTYLY

SEQ ID NO: 8 - protein, 7 aa, Artificial Sequence
  Description of Artificial Sequence Synthetic peptide
  CDR-L2  (Kabat) Antibody 1
LVSKLGS

SEQ ID NO: 9 - protein, 9 aa, Artificial Sequence
  Description of Artificial Sequence Synthetic peptide
  CDR-L3 (Kabat) Antibody 1
VQGTHDPWT

SEQ ID NO: 10 - protein, 6 aa, Artificial Sequence
  Description of Artificial Sequence Synthetic peptide
  CDR-H1 (Kabat) Antibody 1
SGYDWT

SEQ ID NO: 11 - protein, 16 aa, Artificial Sequence
  Description of Artificial Sequence Synthetic peptide
  CDR-H2 (Kabat) Antibody 1
YISYSGNTNYNPSLKS

SEQ ID NO: 12 - protein, 11 aa, Artificial Sequence
  Description of Artificial Sequence Synthetic peptide
  CDR-H3(Kabat) Antibody 1
MMVPHYYVMDA

SEQ ID NO: 13 - protein, 6 aa, Artificial Sequence
  Description of Artificial Sequence Synthetic peptide
  CDR-L1 Antibody 2
SALSYM

SEQ ID NO: 14 - protein, 3 aa, Artificial Sequence
  Description of Artificial Sequence Synthetic peptide
  CDR-L2 Antibody 2
GTS

SEQ ID NO: 15 - protein, 7 aa, Artificial Sequence
  Description of Artificial Sequence Synthetic peptide
  CDR-L3 Antibody 2
HHWSNTQ

SEQ ID NO: 16 - protein, 8 aa, Artificial Sequence
  Description of Artificial Sequence Synthetic peptide
  CDR-H1 Antibody 2
GYTFTNYF

SEQ ID NO: 17 - protein, 9 aa, Artificial Sequence
  Description of Artificial Sequence Synthetic peptide
  CDR-H2 Antibody 2
VDPEQGRAD

SEQ ID NO: 18 - protein, 11 aa, Artificial Sequence
  Description of Artificial Sequence Synthetic peptide
  CDR-H3 Antibody 2
RRAMDNYGFAY

SEQ ID NO: 19 - protein, 10 aa, Artificial Sequence
  Description of Artificial Sequence Synthetic peptide
  CDR-L1  (Kabat) Antibody 2
SANSALSYMY

SEQ ID NO: 20 - protein, 7 aa, Artificial Sequence
  Description of Artificial Sequence Synthetic peptide
  CDR-L2 (Kabat) Antibody 2
GTSNLAS

SEQ ID NO: 21 - protein, 9 aa, Artificial Sequence
  Description of Artificial Sequence Synthetic peptide
  CDR-L3 (Kabat) Antibody 2
HHWSNTQWT

SEQ ID NO: 22 - protein, 5 aa, Artificial Sequence
  Description of Artificial Sequence Synthetic peptide
  CDR-H1 (Kabat) Antibody 2
NYFMN

SEQ ID NO: 23 - protein, 17 aa, Artificial Sequence
  Description of Artificial Sequence Synthetic peptide
  CDR-H2 (Kabat) Antibody 2
RVDPEQGRADYAEKFKK

SEQ ID NO: 24 - protein, 10 aa, Artificial Sequence
  Description of Artificial Sequence Synthetic peptide
  CDR-H3 (Kabat) Antibody 2
RAMDNYGFAY

SEQ ID NO: 25 - protein, 113 aa, Artificial Sequence
  Description of Artificial Sequence Synthetic polypeptide
  VL domain (Antibody 1)
DIVMTQTPLSLSVTPGQPASISCRSSQSLLNSDGNTYLYWYLQKPGQSPQLLIYLVSKLGSGVPNRFSGSGSGTDFTLKISRVEAEDVGVYYCVQGTHDPWTFGGGTKVEIKR

SEQ ID NO: 26 - protein, 121 aa, Artificial Sequence
  Description of Artificial Sequence Synthetic polypeptide
  VH domain (Antibody 1)
QVQLQESGPGLVKPSETLSLTCSVTYHTITSGYDWTWIRKPPGKGMEWIGYISYSGNTNYNPSLKSRVTISRDTSKNQFFLKLSSVTAADTAVYYCASMMVPHYYVMDAWGQGTLVTVSSA

SEQ ID NO: 27 - protein, 219 aa, Artificial Sequence
  Description of Artificial Sequence Synthetic polypeptide
  Light chain (Antibody 1)
DIVMTQTPLSLSVTPGQPASISCRSSQSLLNSDGNTYLYWYLQKPGQSPQLLIYLVSKLGSGVPNRFSGSGSGTDFTLKISRVEAEDVGVYYCVQGTHDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 450 aa, Artificial Sequence
  Description of Artificial Sequence Synthetic polypeptide
  Heavy chain (Antibody 1)
QVQLQESGPGLVKPSETLSLTCSVTYHTITSGYDWTWIRKPPGKGMEWIGYISYSGNTNYNPSLKSRVTISRDTSKNQFFLKLSSVTAADTAVYYCASMMVPHYYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 450 aa, Artificial Sequence
  Description of Artificial Sequence Synthetic polypeptide
  Heavy chain (Antibody 1-S239D, I332E)
QVQLQESGPGLVKPSETLSLTCSVTYHTITSGYDWTWIRKPPGKGMEWIGYISYSGNTNYNPSLKSRVTISRDTSKNQFFLKLSSVTAADTAVYYCASMMVPHYYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 106 aa, Artificial Sequence
  Description of Artificial Sequence Synthetic polypeptide
  Antibody B variable light chain
EIVLTQSPDFQSVTPKEKVTITCSANSALSYMYWYQQKPDQSPKLWVHGTSNLASGVPSRFSGSGSGTDFTLTINSLEAEDAATYYCHHWSNTQWTFGGGTKVEIK

SEQ ID NO: 31 - protein, 119 aa, Artificial Sequence
  Description of Artificial Sequence Synthetic polypeptide
  Antibody B variable heavy chain
QVQLVQSGAEVKKPGSSVKVSCKASGYTFTNYFMNWVRQAPGQGLEWMGRVDPEQGRADYAEKFKKRVTITADKSTSTAYMELSSLRSEDTAVYYCARRAMDNYGFAYWGQGTLVTVSS

SEQ ID NO: 32 - protein, 213 aa, Artificial Sequence
  Description of Artificial Sequence Synthetic polypeptide
  Light chain (Antibody 2)
EIVLTQSPDFQSVTPKEKVTITCSANSALSYMYWYQQKPDQSPKLWVHGTSNLASGVPSRFSGSGSGTDFTLTINSLEAEDAATYYCHHWSNTQWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 449 aa, Artificial Sequence
  Description of Artificial Sequence Synthetic polypeptide
  Heavy chain (Antibody 2)
QVQLVQSGAEVKKPGSSVKVSCKASGYTFTNYFMNWVRQAPGQGLEWMGRVDPEQGRADYAEKFKKRVTITADKSTSTAYMELSSLRSEDTAVYYCARRAMDNYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 449 aa, Artificial Sequence
  Description of Artificial Sequence Synthetic polypeptide
  Heavy chain (Antibody 2-S239D, I332E)
QVQLVQSGAEVKKPGSSVKVSCKASGYTFTNYFMNWVRQAPGQGLEWMGRVDPEQGRADYAEKFKKRVTITADKSTSTAYMELSSLRSEDTAVYYCARRAMDNYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 329 aa, Artificial Sequence
  Description of Artificial Sequence Synthetic polypeptide
  Heavy Chain Constant Domain
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 329 aa, Artificial Sequence
  Description of Artificial Sequence Synthetic polypeptide
  Heavy Chain Constant Domain S239D
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 329 aa, Artificial Sequence
  Description of Artificial Sequence Synthetic polypeptide
  Heavy Chain Constant Domain I332E
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 329 aa, Artificial Sequence
  Description of Artificial Sequence Synthetic polypeptide
  Heavy Chain Constant Domain S239D, I332E
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 106 aa, Artificial Sequence
  Description of Artificial Sequence Synthetic polypeptide
  Light chain Constant Domain
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 107 aa, Artificial Sequence
  Description of Artificial Sequence Synthetic polypeptide
  VL domain (Antibody 2)
EIVLTQSPDFQSVTPKEKVTITCSANSALSYMYWYQQKPDQSPKLWVHGTSNLASGVPSRFSGSGSGTDFTLTINSLEAEDAATYYCHHWSNTQWTFGGGTKVEIKR

SEQ ID NO: 41 - protein, 120 aa, Artificial Sequence
  Description of Artificial Sequence Synthetic polypeptide
  VH domain (Antibody 2)
QVQLVQSGAEVKKPGSSVKVSCKASGYTFTNYFMNWVRQAPGQGLEWMGRVDPEQGRADYAEKFKKRVTITADKSTSTAYMELSSLRSEDTAVYYCARRAMDNYGFAYWGQGTLVTVSSA

(19) United States
(12) Patent Application Publication
(10) Pub. No.: US 2022/0306743 A1
(43) Pub. Date: Sep. 29, 2022

(54) COMBINATION OF CTLA4 AND PD1/PDL1 ANTIBODIES FOR TREATING CANCER

(71) Applicant: Xilio Development, Inc., Waltham, MA (US)

(72) Inventors: Jennifer O'Neil, Waltham, MA (US); Ugur Eskiocak, Waltham, MA (US)

(21) Appl. No.: 17/684,211

(22) Filed: Mar. 1, 2022

Related U.S. Application Data
(60) Provisional application No. 63/155,180, filed on Mar. 1, 2021.

(51) Int. Cl. C07K 16/28; A61P 35/00; A61K 47/68

(52) U.S. Cl. CPC C07K 16/2818; A61P 35/00; A61K 47/6849; C07K 16/2827; A61K 2039/507

(57) ABSTRACT

The invention provides anti-CTLA4 binding proteins (e.g., antibodies, bispecific antibodies, and chimeric receptors) and their use in combination with PD-1 signaling agents (e.g., PD-1 inhibitors or PD-L1 inhibitors) for treating and preventing cancer, as well as compositions and kits comprising the anti-CTLA4 binding proteins and PD-1 signaling agents.

This application claims priority to, and the benefit of, U.S. provisional application No. 63/155,180, filed on Mar. 1, 2021, the contents of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Jun. 8, 2022, is named XTX_CTLA4_04US1_SL.txt and is 46,947 bytes in size.

### FIELD OF THE INVENTION

This invention relates to anti-cytotoxic T-lymphocyte-associated protein 4(CTLA4) binding proteins (e.g., anti-CTLA4 antibodies) and methods related to use of the same in combination with a PD-1 signaling agents (e.g. PD-1 or PD-L1 inhibitors).

### BACKGROUND OF THE INVENTION

Cancer is the second leading cause of death in the United States, accounting for more deaths than the next five leading causes (chronic respiratory disease, stroke, accidents, Alzheimer's disease and diabetes). While great strides have been made especially with targeted therapies, there remains a great deal of work to do in this space. Immunotherapy and a branch of this field, immuno-oncology, is creating viable and exciting therapeutic options for treating malignancies. Specifically, it is now recognized that one hallmark of cancer is immune evasion and significant efforts have identified targets and developed therapies to these targets to reactivate the immune system to recognize and treat cancer. The anti-cytotoxic T-lymphocyte-associated protein 4 (CTLA4) antibody, ipilimumab, has led to long-term survival of patients suffering from stage III/IV malignant melanoma. Ipilimumab is an immune checkpoint antagonist that interrupts the inhibition of T cells by blocking CTLA4, and may lead to the depletion of T Regulatory cells (Treg) (Korman, A., et al., 2005. Tumor immunotherapy: preclinical and clinical activity of anti-CTLA4 antibodies. Current Opinion in Investigational Drugs 6:582-591; Quezada et al., J. Exp. Med., 206(8):1717-1725, 2009; Selby et al. Cancer Immunol Res., 1(1); 32-42, 2013. Unfortunately, ipilimumab causes generalized (not tumor-specific) activation of T-cell dependent immune responses that leads to immune-related adverse effects which can be life-threatening and are often dose and treatment duration-limiting (Weber, J. S., et al., 2008. Phase I/II study of ipilimumab for patients with metastatic melanoma. Journal of Clinical Oncology 26:5950-5956). These include enterocolitis, dermatitis, hypophysitis, uveitis, hepatitis, nephritis and death. Enterocolitis is the most common major toxicity (affecting approximately 20% of patients). The severe safety risks related to immune-mediated adverse reactions prompted the FDA to approve ipilimumab with a Risk Evaluation and Mitigation Strategy (REMS). Recently, coadministration of ipilimumab and a second immune checkpoint modulator targeting PD1 (e.g., nivolumab) has been shown to significantly increase efficacy of immunotherapy of melanoma when compared to ipilimumab alone. This gain, however, was associated with increased frequencies of grade 3/4 adverse effects, which affected more than 50% of patients receiving combination treatment (Wolchok, J. D., et al. 2013. Nivolumab plus Ipilimumab in Advanced Melanoma. N Engl J Med).

These findings illustrate the need for developing anti-CTLA4 protein therapeutics that effectively target tumors without the side effects associated with certain anti-CTLA4 antibodies such as ipilimumab. Provided herein are anti-CTLA binding proteins, compositions thereof and methods of use thereof for addressing this need.

All references cited herein, including patent applications, patent publications, and scientific literature, are herein incorporated by reference in their entirety, as if each individual reference were specifically and individually indicated to be incorporated by reference.

### SUMMARY OF THE INVENTION

Provided herein are anti-cytotoxic T-lymphocyte-associated protein 4(CTLA4) binding proteins for use in combination with PD-1 signaling agents (e.g., PD-1 or PD-L1 inhibitors), and compositions thereof, and methods of using the same.

In one aspect, the present invention provides, a method of treating cancer in a subject, the method comprising administering to the subject: (a) an anti-CTLA4 antibody comprising a heavy chain variable region (vH) CDR1 comprising NYFMN (SEQ ID NO: 22), a vH CDR2 comprising RVDPEQGRADYAEKFKK (SEQ ID NO: 23), a vH CDR3 comprising RAMDNYGFAY (SEQ ID NO: 24); and a light chain variable region (vL) CDR1 comprising SANSALSYMY (SEQ ID NO: 19), a vL CDR2 comprising GTSNLAS (SEQ ID NO: 20), a vL CDR3 comprising HHWSNTQWT (SEQ ID NO: 21); and (b) a PD-1 or PD-L1 inhibitor.

In some embodiments, the anti-CTLA4 antibody is administered at an effective dose of between about 0.1-20 mg/kg. In some embodiments, the anti-CTLA4 antibody is administered at an effective dose of between about 0.1-15 mg/kg, 0.1-10 mg/kg, 0.3-10 mg/kg, or 0.3-15 mg/kg. In some embodiments, the effective dose of the anti-CTLA4 antibody is selected from 0.3 mg/kg, 1 mg/kg, 3 mg/kg, 10 mg/kg and 20 mg/kg.

In some embodiments, the anti-CTLA4 antibody is administered at a low dose. In some embodiments, the anti-CTLA4 antibody is administered at a dose between 0.01-1 mg/kg. In some embodiments, the anti-CTLA4 antibody is administered at a dose between 0.01-3 mg/kg. In some embodiments, the anti-CTLA4 antibody administered at a dose selected from 0.01 mg/kg, 0.03 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 3 mg/kg.

In some embodiments, the effective dose of the anti-CTLA4 antibody is 0.01-1 mg/kg, 0.01-3 mg, kg, 1-100 mg, 10-100 mg, 20-100 mg, 30-100,50-100,4-100 mg, 4-200 mg, 4-300 mg, 4-400 mg, 4-500 mg, 4-600 mg, 4-700 mg, 4-800 mg, 4-900 mg, 10-100 mg, 10-200 mg, 10-300 mg, 10-400 mg, 10-500 mg, 10-600 mg, 10-700 mg, 10-800 mg, 10-900 mg, 10-1000 mg, 100-300 mg, 300-500 mg, 500-700 mg, 700-900 mg, or 800-1000 mg.

In some embodiments, the anti-CTLA4 antibody comprises a heavy chain constant domain comprising amino acid substitutions S239D or I332E or both, wherein the amino acid residues are numbered according to the EU index as in Kabat.

In some embodiments, the anti-CTLA4 antibody comprises a vH that is at least 90% identical to SEQ ID NO: 31.

In some embodiments, the anti-CTLA4 antibody comprises a vL that is at least 90% identical to SEQ ID NO: 30.

In some embodiments, the anti-CTLA4 antibody is afucosylated or fucose-deficient.

In some embodiments, the anti-CTLA4 antibody or antigen-binding fragment thereof is conjugated to an agent. In some embodiments, the agent is an inhibitor of tubulin polymerization, a DNA damaging agent, or a DNA synthesis inhibitor. In some embodiments, the agent is a maytansinoid, an auristatin, a pyrrolobenzodiazepine (PBD) dimer, a calichcamicin, a duocarmycin, an indo-linobenzodiazepine dimer, or exatecan derivative Dxd.

In some embodiments, the PD-1/PD-L1 inhibitor is an antibody.

In some embodiments, the PD-1/PD-L1 inhibitor is a PD-1 antibody.

In some embodiments, the anti-PD-1 antibody is selected from nivolumab, pembrolizumab, and cemiplimab.

In some embodiments, PD-1 antibody is administered at an effective dose of between 1-10 mg/kg. In some embodiments, the effective dose of the PD-1 antibody is 10 mg/kg. In some embodiments, the effective dose of the PD-1 antibody is 4-1000 mg. In some embodiments, the effective dose of the PD-1 antibody is 4-100 mg, 4-200 mg, 4-300 mg, 4-400 mg, 4-500 mg, 4-600 mg, 4-700 mg, 4-800 mg, 4-900 mg, 10-100 mg, 10-200 mg, 10-300 mg, 10-400 mg, 10-500 mg, 10-600 mg, 10-700 mg, 10-800 mg, 10-900 mg, 10-1000 mg, 100-300 mg, 300-500 mg, 500-700 mg, 700-900 mg, or 800-1000 mg.

In some embodiments, the anti-PD-1 antibody is administered at an effective dose of 200 mg.

In some embodiments, the anti-PD-1 antibody is administered weekly, every other week, every 3 weeks, every 4 weeks, every 5 weeks, every 6 weeks, or monthly.

In some embodiments, the anti-PD-1 antibody is administered every 3 weeks.

In some embodiments, the PD-1/PD-L1 inhibitor is a PD-L1 antibody.

In some embodiments, the anti-PD-L1 antibody is selected from atezolizumab, avelumab, durvalumab.

In some embodiments, the anti-PD-L1 antibody is administered at an effective dose of between 200-2000 mg.

In some embodiments, the anti-PD-L1 antibody is administered weekly, every other week, every 3 weeks, every 4, weeks, every 6 weeks or monthly.

In some embodiments, the anti-PD-1 and anti-CTLA4 antibodies are formulated for intravenous administration.

In some embodiments, the anti-PD-1 and anti-CTLA4 antibodies are formulated together in the same composition. In some embodiments, the anti-PD-1 and anti-CTLA4 antibodies are formulated separately.

In some embodiments, the anti-PD-1 antibody is administered concurrently with the anti-CTLA4 antibody.

In some embodiments, the cancer is leukemia, lymphoma, head and neck cancer, colorectal cancer, prostate cancer, pancreatic cancer, melanoma, myeloma, breast cancer, neuroblastoma, lung cancer, ovarian cancer, osteosarcoma, bladder cancer, cervical cancer, liver cancer, kidney cancer, skin cancer, testicular cancer, or cutaneous squamous cell carcinoma (CSCC).

In some embodiments, the cancer is lung cancer. In some embodiments, the lung cancer is small-cell lung carcinoma (SCLC) or non-small-cell lung carcinoma (NSCLC). In some embodiments, the cancer is melanoma.

In some embodiments, the cancer melanoma, non-small cell lung cancer (NSCLC), pleural mesothelioma, kidney cancer, liver cancer, or colorectal cancer.

Provided herein are anti-CTLA4 antibodies or antigen-binding fragment thereof comprising a light chain variable (VL) domain and a heavy chain variable (VH) domain, wherein: the VL domain comprises a CDR-L1 comprising the amino acid sequence of SEQ ID NO: 1, a CDR-L2 comprising the amino acid sequence of SEQ ID NO: 2, and a CDR-L3 comprising the amino acid sequence of SEQ ID NO: 3; and/or the VH domain comprises a CDR-H1 comprising the amino acid sequence of SEQ ID NO: 4, a CDR-H2 comprising the amino acid sequence of SEQ ID NO: 5, and a CDR-H3 comprising the amino acid sequence of SEQ ID NO: 6; or the VL domain comprises a CDR-L1 comprising the amino acid sequence of SEQ ID NO: 13, a CDR-L2 comprising the amino acid sequence of SEQ ID NO: 14, and a CDR-L3 comprising the amino acid sequence of SEQ ID NO: 15; and/or the VH domain comprises a CDR-H1 comprising the amino acid sequence of SEQ ID NO: 16, a CDR-H2 comprising the amino acid sequence of SEQ ID NO: 17, and a CDR-H3 comprising the amino acid sequence of SEQ ID NO: 18; or the VL domain comprises a CDR-L1 comprising the amino acid sequence of SEQ ID NO: 7, a CDR-L2 comprising the amino acid sequence of SEQ ID NO: 8, and a CDR-L3 comprising the amino acid sequence of SEQ ID NO: 9; and/or the VH domain comprises a CDR-H1 comprising the amino acid sequence of SEQ ID NO: 10, a CDR-H2 comprising the amino acid sequence of SEQ ID NO: 11, and a CDR-H3 comprising the amino acid sequence of SEQ ID NO: 12; or the VL domain comprises a CDR-L1 comprising the amino acid sequence of SEQ ID NO: 19, a CDR-L2 comprising the amino acid sequence of SEQ ID NO: 20, and a CDR-L3 comprising the amino acid sequence of SEQ ID NO: 21; and/or the VH domain comprises a CDR-H1 comprising the amino acid sequence of SEQ ID NO: 22, a CDR-H2 comprising the amino acid sequence of SEQ ID NO: 23, and a CDR-H3 comprising the amino acid sequence of SEQ ID NO: 24.

Provided herein are anti-CTLA4 antibodies or antigen-binding fragment thereof comprising a light chain variable (VL) domain and a heavy chain variable (VH) domain, wherein the VL domain comprises a CDR-L1 comprising the amino acid sequence of SEQ ID NO: 1, a CDR-L2 comprising the amino acid sequence of SEQ ID NO: 2, and a CDR-L3 comprising the amino acid sequence of SEQ ID NO: 3; and/or the VH domain comprises a CDR-H1 comprising the amino acid sequence of SEQ ID NO: 4, a CDR-H2 comprising the amino acid sequence of SEQ ID NO: 5, and a CDR-H3 comprising the amino acid sequence of SEQ ID NO: 6.

Provided herein are anti-CTLA4 antibodies or antigen-binding fragment thereof comprising a light chain variable (VL) domain and a heavy chain variable (VH) domain, wherein the VL domain comprises a CDR-L1 comprising the amino acid sequence of SEQ ID NO: 13, a CDR-L2 comprising the amino acid sequence of SEQ ID NO: 14, and a CDR-L3 comprising the amino acid sequence of SEQ ID NO: 15; and/or the VH domain comprises a CDR-H1 comprising the amino acid sequence of SEQ ID NO: 16, a CDR-H2 comprising the amino acid sequence of SEQ ID NO: 17, and a CDR-H3 comprising the amino acid sequence of SEQ ID NO: 18.

Provided herein are anti-CTLA4 antibodies or antigen-binding fragment thereof comprising a light chain variable (VL) domain and a heavy chain variable (VH) domain, wherein the VL domain comprises a CDR-L1 comprising the amino acid sequence of SEQ ID NO: 7, a CDR-L2 comprising the amino acid sequence of SEQ ID NO: 8, and a CDR-L3 comprising the amino acid sequence of SEQ ID NO: 9; and/or the VH domain comprises a CDR-H1 comprising the amino acid sequence of SEQ ID NO: 10, a CDR-H2 comprising the amino acid sequence of SEQ ID NO: 11, and a CDR-H3 comprising the amino acid sequence of SEQ ID NO: 12.

Provided herein are anti-CTLA4 antibodies or antigen-binding fragment thereof comprising a light chain variable (VL) domain and a heavy chain variable (VH) domain, wherein the VL domain comprises a CDR-L1 comprising the amino acid sequence of SEQ ID NO: 19, a CDR-L2 comprising the amino acid sequence of SEQ ID NO: 20, and a CDR-L3 comprising the amino acid sequence of SEQ ID NO: 21; and/or the VH domain comprises a CDR-H1 comprising the amino acid sequence of SEQ ID NO: 22, a CDR-H2 comprising the amino acid sequence of SEQ ID NO: 23, and a CDR-H3 comprising the amino acid sequence of SEQ ID NO: 24.

In some of any of the provided of the provided embodiments, the VL domain comprises an amino acid sequence that has at least about 90% sequence identity to the amino acid sequence of SEQ ID NO: 25, and the VH domain comprises an amino acid sequence that has at least about 90% sequence identity to the amino acid sequence of SEQ ID NO: 26; or the VL domain comprises an amino acid sequence that has at least about 90% sequence identity to the amino acid sequence of SEQ ID NO: 30, and the VH domain comprises an amino acid sequence that has at least about 90% sequence identity to the amino acid sequence of SEQ ID NO: 31.

In some of any of the provided embodiments, the VL domain comprises an amino acid sequence that has at least about 90% sequence identity to the amino acid sequence of SEQ ID NO: 25, and the VH domain comprises an amino acid sequence that has at least about 90% sequence identity to the amino acid sequence of SEQ ID NO: 26. In some of any of the provided embodiments, the VL domain comprises an amino acid sequence that has at least about 90% sequence identity to the amino acid sequence of SEQ ID NO: 30, and the VH domain comprises an amino acid sequence that has at least about 90% sequence identity to the amino acid sequence of SEQ ID NO: 31.

In some of any of the provided embodiments, the VL domain comprises the amino acid sequence of SEQ ID NO: 25, and the VH domain comprises the amino acid sequence of SEQ ID NO: 26; or the VL domain comprises the amino acid sequence of SEQ ID NO: 30, and the VH domain comprises the amino acid sequence of SEQ ID NO: 31.

Provided herein are anti-CTLA4 antibodies or antigen-binding fragment thereof comprising a light chain variable (VL) domain and a heavy chain variable (VH) domain, wherein: the VL domain comprises the amino acid sequence of SEQ ID NO: 25 and/or the VH domain comprises the amino acid sequence of SEQ ID NO: 26; or the VL domain comprises the amino acid sequence of SEQ ID NO: 30 and/or the VH domain comprises the amino acid sequence of SEQ ID NO: 31.

In some of any of the provided embodiments, the VL domain comprises the amino acid sequence of SEQ ID NO: 25, and the VH domain comprises the amino acid sequence of SEQ ID NO: 26. In some of any of the provided embodiments, the VL domain comprises the amino acid sequence of SEQ ID NO: 30, and the VH domain comprises the amino acid sequence of SEQ ID NO: 31.

In some of any of the provided embodiments, the antibody or antigen-binding fragment thereof comprises a heavy chain constant domain (CH). In some of any of the provided embodiments, the antibody or antigen-binding fragment thereof comprises a CH sequence selected from the group consisting of SEQ ID NOS: 35-38. In some of any of the provided embodiments, the CH comprises amino acid substitutions S239D or I332E or both, wherein the amino acid residues are numbered according to the EU index as in Kabat. In some of any of the provided embodiments, the antibody or antigen-binding fragment thereof comprises a CH sequence of SEQ ID NO: 38.

In some of any embodiments, the antibody or antigen-binding fragment thereof comprises a light chain constant domain (CL). In some of any embodiments, the antibody or antigen-binding fragment thereof comprises a CL sequence of SEQ ID NO:39.

In some of any embodiments, the light chain comprises the amino acid sequence of SEQ ID NO: 27, and the heavy chain comprises the amino acid sequence of SEQ ID NO: 29; or the light chain comprises the amino acid sequence of SEQ ID NO: 32, and the heavy chain comprises the amino acid sequence of SEQ ID NO: 34.

Provided herein are anti-CTLA4 antibodies or antigen-binding fragment thereof comprising a light chain comprising the amino acid sequence of SEQ ID NO: 27 and a heavy chain comprising the amino acid sequence of SEQ ID NO: 29.

Provided herein are anti-CTLA4 antibodies or antigen-binding fragment thereof comprising a light chain comprising the amino acid sequence of SEQ ID NO: 32 and a heavy chain comprising the amino acid sequence of SEQ ID NO: 34.

In some of any embodiments, the antibody or antigen-binding fragment thereof is afucosylated or fucose-deficient.

In some of any embodiments, the anti-CTLA4 antibody or antigen-binding fragment thereof is conjugated to an agent. In some of any embodiments, the agent is an inhibitor of tubulin polymerization, a DNA damaging agent, or a DNA synthesis inhibitor. In some of any embodiments, the agent is a maytansinoid, an auristatin, a pyrrolobenzodiazepine (PBD) dimer, a calicheamicin, a duocarmycin, a indo-linobenzodiazepine dimer, or exatecan derivative Dxd.

Provided herein are bispecific antibodies or an antigen-binding fragment thereof, comprising a light chain and a heavy chain of a first pair that specifically binds to CTLA4; a light chain and a heavy chain of a second pair that specifically binds to an antigen; wherein the light chain of the first pair comprises a VL domain, and the heavy chain of the first pair comprises a VH domain and wherein: the VL domain comprises a CDR-L1 comprising the amino acid sequence of SEQ ID NO: 1, a CDR-L2 comprising the amino acid sequence of SEQ ID NO: 2, and a CDR-L3 comprising the amino acid sequence of SEQ ID NO: 3; and/or the VH domain comprises a CDR-H1 comprising the amino acid sequence of SEQ ID NO: 4, a CDR-H2 comprising the amino acid sequence of SEQ ID NO: 5, and a CDR-H3 comprising the amino acid sequence of SEQ ID NO: 6; or the VL domain comprises a CDR-L1 comprising the amino acid sequence of SEQ ID NO: 13, a CDR-L2 comprising the amino acid sequence of SEQ ID NO: 14, and a CDR-L3 comprising the amino acid sequence of SEQ ID NO: 15; and/or the VH domain comprises a CDR-H1 comprising the amino acid sequence of SEQ ID NO: 16, a CDR-H2 comprising the amino acid sequence of SEQ ID NO: 17, and a CDR-H3 comprising the amino acid sequence of SEQ ID NO: 18; or the VL domain comprises a CDR-L1 comprising the amino acid sequence of SEQ ID NO: 7, a CDR-L2 comprising the amino acid sequence of SEQ ID NO: 8, and a CDR-L3 comprising the amino acid sequence of SEQ ID NO: 9; and/or the VH domain comprises a CDR-H1 comprising the amino acid sequence of SEQ ID NO: 10, a CDR-H2 comprising the amino acid sequence of SEQ ID NO: 11, and a CDR-H3 comprising the amino acid sequence of SEQ ID NO: 12; or the VL domain comprises a CDR-L1 comprising the amino acid sequence of SEQ ID NO: 19, a CDR-L2 comprising the amino acid sequence of SEQ ID NO: 20, and a CDR-L3 comprising the amino acid sequence of SEQ ID NO: 21; and/or the VH domain comprises a CDR-H1 comprising the amino acid sequence of SEQ ID NO: 22, a CDR-H2 comprising the amino acid sequence of SEQ ID NO: 23, and a CDR-H3 comprising the amino acid sequence of SEQ ID NO: 24.

Provided herein are bispecific antibodies or an antigen-binding fragment thereof, comprising a light chain and a heavy chain of a first pair that specifically binds to CTLA4; a light chain and a heavy chain of a second pair that specifically binds to an antigen; wherein the light chain of the first pair comprises a VL domain, and the heavy chain of the first pair comprises a VH domain and wherein the VL domain comprises a CDR-L1 comprising the amino acid sequence of SEQ ID NO: 1, a CDR-L2 comprising the amino acid sequence of SEQ ID NO: 2, and a CDR-L3 comprising the amino acid sequence of SEQ ID NO: 3; and/or the VH domain comprises a CDR-H1 comprising the amino acid sequence of SEQ ID NO: 4, a CDR-H2 comprising the amino acid sequence of SEQ ID NO: 5, and a CDR-H3 comprising the amino acid sequence of SEQ ID NO: 6.

Provided herein are bispecific antibodies or an antigen-binding fragment thereof, comprising a light chain and a heavy chain of a first pair that specifically binds to CTLA4; a light chain and a heavy chain of a second pair that specifically binds to an antigen; wherein the light chain of the first pair comprises a VL domain, and the heavy chain of the first pair comprises a VH domain and wherein the VL domain comprises a CDR-L1 comprising the amino acid sequence of SEQ ID NO: 13, a CDR-L2 comprising the amino acid sequence of SEQ ID NO: 14, and a CDR-L3 comprising the amino acid sequence of SEQ ID NO: 15; and/or the VH domain comprises a CDR-H1 comprising the amino acid sequence of SEQ ID NO: 16, a CDR-H2 comprising the amino acid sequence of SEQ ID NO: 17, and a CDR-H3 comprising the amino acid sequence of SEQ ID NO: 18.

Provided herein are bispecific antibodies or an antigen-binding fragment thereof, comprising a light chain and a heavy chain of a first pair that specifically binds to CTLA4; a light chain and a heavy chain of a second pair that specifically binds to an antigen; wherein the light chain of the first pair comprises a VL domain, and the heavy chain of the first pair comprises a VH domain and wherein the VL domain comprises a CDR-L1 comprising the amino acid sequence of SEQ ID NO: 7, a CDR-L2 comprising the amino acid sequence of SEQ ID NO: 8, and a CDR-13 comprising the amino acid sequence of SEQ ID NO: 9; and/or the VH domain comprises a CDR-H1 comprising the amino acid sequence of SEQ ID NO: 10, a CDR-H2 comprising the amino acid sequence of SEQ ID NO: 1, and a CDR-113 comprising the amino acid sequence of SEQ ID NO: 12.

Provided herein are bispecific antibodies or an antigen-binding fragment thereof, comprising a light chain and a heavy chain of a first pair that specifically binds to CTLA4; a light chain and a heavy chain of a second pair that specifically binds to an antigen; wherein the light chain of the first pair comprises a VL domain, and the heavy chain of the first pair comprises a VH domain and wherein the VL domain comprises a CDR-L1 comprising the amino acid sequence of SEQ ID NO: 19, a CDR-L2 comprising the amino acid sequence of SEQ ID NO: 20, and a CDR-L3 comprising the amino acid sequence of SEQ ID NO: 21; and/or the VH domain comprises a CDR-H1 comprising the amino acid sequence of SEQ ID NO: 22, a CDR-H2 comprising the amino acid sequence of SEQ ID NO: 23, and a CDR-H3 comprising the amino acid sequence of SEQ ID NO: 24.

In some of any embodiments, the VL domain comprises an amino acid sequence that has at least about 90% sequence identity to the amino acid sequence of SEQ ID NO: 25, and the VH domain comprises an amino acid sequence that has at least about 90% sequence identity to the amino acid sequence of SEQ ID NO: 26; or the VL domain comprises an amino acid sequence that has at least about 90% sequence identity to the amino acid sequence of SEQ ID NO: 30, and the VH domain comprises an amino acid sequence that has at least about 90% sequence identity to the amino acid sequence of SEQ ID NO: 31.

Provided herein are bispecific antibodies or an antigen-binding fragment thereof, comprising a light chain and a heavy chain of a first pair that specifically binds to CTLA4; a light chain and a heavy chain of a second pair that specifically binds to an antigen; wherein the light chain of the first pair comprises a VL domain, and the heavy chain of the first pair comprises a VH domain and wherein: the VL domain comprises the amino acid sequence of SEQ ID NO: 25 and/or the VH domain comprises the amino acid sequence of SEQ ID NO: 26; or the VL domain comprises the amino acid sequence of SEQ ID NO: 30 and/or the VH domain comprises the amino acid sequence of SEQ ID NO: 31.

In some of any embodiments, the VL domain comprises the amino acid sequence of SEQ ID NO: 25, and the VH domain comprises the amino acid sequence of SEQ ID NO: 26; or the VL domain comprises the amino acid sequence of SEQ ID NO: 30, and the VH domain comprises the amino acid sequence of SEQ ID NO: 31.

In some of any embodiments, the bispecific antibody or antigen-binding fragment thereof comprises a heavy chain constant domain (CH). In some of any embodiments, the bispecific antibody or antigen-binding fragment thereof comprises a CH sequence selected from the group consisting of SEQ ID NOS: 35-38. In some of any embodiments, the CH comprises amino acid substitutions S239D or I332E or both, wherein the amino acid residues are numbered according to the EU index as in Kabat. In some of any embodiments, the bispecific antibody or antigen-binding fragment thereof comprises a CH sequence of SEQ ID NO: 38.

In some of any embodiments, the bispecific antibody or antigen-binding fragment thereof comprises a light chain constant domain (CL). In some of any embodiments, the bispecific antibody or antigen-binding fragment thereof comprises a CL sequence of SEQ ID NO:39.

In some of any embodiments, the light chain comprises the amino acid sequence of SEQ ID NO: 27, and the heavy chain comprises the amino acid sequence of SEQ ID NO: 29; or the light chain comprises the amino acid sequence of SEQ ID NO: 32, and the heavy chain comprises the amino acid sequence of SEQ ID NO: 34.

Provided herein are bispecific antibodies or an antigen-binding fragment thereof, comprising a light chain and a heavy chain of a first pair that specifically binds to CTLA4; a light chain and a heavy chain of a second pair that specifically binds to an antigen; wherein the first pair comprises a light chain comprising the amino acid sequence of SEQ ID NO: 27 and a heavy chain comprising the amino acid sequence of SEQ ID NO: 29.

Provided herein are bispecific antibodies or an antigen-binding fragment thereof, comprising a light chain and a heavy chain of a first pair that specifically binds to CTLA4; a light chain and a heavy chain of a second pair that specifically binds to an antigen; wherein the first pair comprises a light chain comprising the amino acid sequence of SEQ ID NO: 32 and a heavy chain comprising the amino acid sequence of SEQ ID NO: 34.

In some of any embodiments, the bispecific antibody or antigen-binding fragment thereof is afucosylated or fucose-deficient.

In some of any embodiments, the bispecific antibody or antigen-binding fragment thereof is conjugated to an agent. In some of any embodiments, the agent is an inhibitor of tubulin polymerization, a DNA damaging agent, or a DNA synthesis inhibitor. In some of any embodiments, the agent is a maytansinoid, an auristatin, a pyrrolobenzodiazepine (PBD) dimer, a calicheamicin, a duocarmycin, a indo-linobenzodiazepine dimer, or exatecan derivative Dxd.

Also provided herein are nucleic acids encoding any of the provided anti-CTLA4 antibody or antigen-binding fragment thereof or any of the provided bispecific antibody or antigen-binding fragment thereof.

Also provided are vectors comprising any of the nucleic acids provided herein.

Also provided are host cells comprising any of the provided anti-CTLA4 antibody or antigen-binding fragment thereof, any of the provided bispecific antibody or antigen-binding fragment thereof, or any of the nucleic acids provided herein.

In some of any such embodiments, the host cell is capable of producing an antibody or antigen-binding fragment thereof that is afucosylated or fucose-deficient. In some of any such embodiments, the host cell has an alpha1,6-fucosyltransferase (Fut8) knockout. In some of any such embodiments, the host cell overexpresses β1,4-N-acetylglycosminyltransferase III (GnT-III). In some of any such embodiments, the host cell overexpresses Golgi μ-mannosidase I (ManII).

Also provided are methods of producing an antibody or antigen-binding fragment thereof, that involve culturing any of the provided host cells under conditions that produce the antibody or antigen-binding fragment thereof.

Also provided are methods of producing an afucosylated or fucose-deficient antibody or antigen-binding fragment thereof, that involve culturing any of the provided host cells under conditions that produce the antibody or antigen-binding fragment thereof.

In some of any such embodiments, the methods also involve recovering the antibody or antigen-binding fragment thereof produced by the host cell.

Also provided are antibodies or antigen-binding fragment thereof produced by any of the methods of producing antibodies or antigen-binding fragment thereof provided herein.

Also provided are compositions comprising any of the provided anti-CTLA4 antibody or antigen-binding fragment thereof, any of the provided bispecific antibody or antigen-binding fragment thereof or any of the provided antibody or antigen-binding fragment thereof.

Also provided are pharmaceutical compositions comprising any of the provided anti-CTLA4 antibody or antigen-binding fragment thereof, any of the provided bispecific antibody or antigen-binding fragment thereof or any of the provided antibody or antigen-binding fragment thereof and a pharmaceutically acceptable carrier.

Also provided are pharmaceutical compositions comprising any of the provided anti-CTLA4 antibody or antigen-binding fragment thereof, any of the provided bispecific antibody or antigen-binding fragment thereof or any of the provided antibody or antigen-binding fragment thereof and a pharmaceutically acceptable carrier, for use in treating or preventing a neoplastic disease in a subject.

Also provided are uses of a pharmaceutical composition comprising any of the provided anti-CTLA4 antibody or antigen-binding fragment thereof, any of the provided bispecific antibody or antigen-binding fragment thereof or any of the provided antibody or antigen-binding fragment thereof and a pharmaceutically acceptable carrier, in the manufacture of a medicament for treating or preventing a neoplastic disease in a subject.

Also provided are kits comprising any of the provided anti-CTLA4 antibody or antigen-binding fragment thereof, any of the provided bispecific antibody or antigen-binding fragment thereof or any of the provided antibody or antigen-binding fragment thereof.

Also provided are method of treating or preventing a neoplastic disease in a subject, the method involving administering to a subject an effective amount any of the provided anti-CTLA4 antibody or antigen-binding fragment thereof, any of the provided bispecific antibody or antigen-binding fragment thereof, any of the provided antibody or antigen-binding fragment thereof or any of the provided compositions.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### DETAILED DESCRIPTION

Therapeutics such as checkpoint inhibitors demonstrate unprecedented responses in cancer but their use is limited by immune-related adverse events (irAEs) and other toxicities (e.g., hypophysitis). Provided herein are protein therapeutics that bind CTLA4, for example in a tumor microenvironment, to achieve increased durable response rates and improved safety profiles. Improved binding affinity, increased functional activity such as ADCC, and other advantages as described herein of the provided CTLA4-binding proteins, such as antibodies, bispecific antibodies or antigen-binding fragments thereof, can lead to improved response to the therapy and improved safety profiles, such as reducing or minimizing adverse events that can be associated with certain immunotherapies.

### I. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “an antibody” optionally includes a combination of two or more such antibodies, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

The term “antibody” includes polyclonal antibodies, monoclonal antibodies (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)2, and Fv). The term “immunoglobulin” (Ig) is used interchangeably with “antibody” herein.

The basic 4-chain antibody unit is a heterotetrameric glycoprotein composed of two identical light (L) chains and two identical heavy (H) chains. An IgM antibody consists of S of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for p and a isotypes. Each L chain has at the N-terminus, a variable domain (VL) followed by a constant domain at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see e.g., Basic and Clinical Immunology, 8th Edition, Daniel P. Sties. Abba I. Terr and Tristram G. Parsolw (eds), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the CH sequence and function, e.g., humans express the following subclasses: IgG1, IgG2, IgG3, IgG4, IgA1 and IgA2, IgG1 antibodies can exist in multiple polymorphic variants termed allotypes (reviewed in Jefferis and Lefranc 2009. mAbs Vol 1 Issue 4 1-7) any of which are suitable for use in the invention. Common allotypic variants in human populations are those designated by the letters a,f,n,z.

An “isolated” antibody is one that has been identified, separated and/or recovered from a component of its production environment (e.g., naturally or recombinantly). In some embodiments, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In some embodiments, the polypeptide is purified: (1) to greater than 95% by weight of antibody as determined by, for example, the Lowry method, and in some embodiments, to greater than 99% by weight; (1) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or silver stain. Isolated antibody includes the antibody in situ within recombinant cells since at least one component of the antibody's natural environment will not be present. Ordinarily, however, an isolated polypeptide or antibody is prepared by at least one purification step.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. In some embodiments, monoclonal antibodies have a C-terminal cleavage at the heavy chain and/or light chain. For example, 1, 2, 3, 4, or 5 amino acid residues are cleaved at the C-terminus of heavy chain and/or light chain. In some embodiments, the C-terminal cleavage removes a C-terminal lysine from the heavy chain. In some embodiments, monoclonal antibodies have an N-terminal cleavage at the heavy chain and/or light chain. For example, 1, 2, 3, 4, or 5 amino acid residues are cleaved at the N-terminus of heavy chain and/or light chain.

In some embodiments truncated forms of monoclonal antibodies can be made by recombinant techniques. In some embodiments, monoclonal antibodies are highly specific, being directed against a single antigenic site. In some embodiments, monoclonal antibodies are highly specific, being directed against multiple antigenic sites (such as a bispecific antibody or a multispecific antibody). The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method, recombinant DNA methods, phage-display technologies, and technologies for producing human or human-like antibodies in animals that have parts or all of the human immunoglobulin loci or genes encoding human immunoglobulin sequences.

The term “naked antibody” refers to an antibody that is not conjugated to a cytotoxic moiety or radiolabel.

The term “parental antibody” refers to an antibody prior to modification.

An “antibody-drug conjugate” or “ADC” refers to an antibody conjugated to one or more heterologous molecule(s), including but not limited to a cytotoxic agent.

The terms “full-length antibody,” “intact antibody” or “whole antibody” are used interchangeably to refer to an antibody in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, the antigen binding and/or the variable region of the intact antibody. Examples of antigen-binding antibody fragments include domain antibodies (dAbs), Fab, Fab′, F(ab′)2 and Fv fragments; diabodies; linear antibodies (see U.S. Pat. No. 5,641,870. Example 2; Zapata et al., Protein Eng. 8(10): 1057-1062 [1995]); single-chain antibody molecules, and multispecific antibodies formed from antibody fragments. Single heavy chain antibodies or single light chain antibodies can be engineered, or in the case of the heavy chain, can be isolated from camelids, shark, libraries or mice engineered to produce single heavy chain molecules.

Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having different antigen-binding activity and is still capable of cross-linking antigen. Fab′ fragments differ from Fab fragments by having a few additional residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences and glycan in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. In some embodiments, the sFv polypeptide further comprises a polypeptide linker between the VH and VL domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Functional fragments” of the antibodies of the invention comprise a portion of an intact antibody, generally including the antigen binding or variable region of the intact antibody or the Fv region of an antibody which retains or has modified FcR binding capability. Examples of antibody fragments include linear antibody, single-chain antibody molecules and multispecific antibodies formed from antibody fragments.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATIZED antibodies wherein the antigen-binding region of the antibody is derived from an antibody produced by, e.g., immunizing macaque monkeys with an antigen of interest. As used herein, “humanized antibody” is used as a subset of “chimeric antibodies.”

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as murine, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. In some embodiments, the number of these amino acid substitutions in the FR are no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409. In some embodiments, humanized antibodies are directed against a single antigenic site. In some embodiments, humanized antibodies are directed against multiple antigenic sites. An alternative humanization method is described in U.S. Pat. No. 7,981,843 and U.S. Patent Application Publication No. 2006/0134098.

The “variable region” or “variable domain” of an antibody refers to the amino-terminal domains of the heavy or light chain of the antibody. Accordingly, the terms “variable region” and “variable domain” as used herein may be used interchangeably. The variable domains of the heavy chain and light chain may be referred to as “VH” and “VL”, respectively. These domains are generally the most variable parts of the antibody (relative to other antibodies of the same class) and contain the antigen binding sites. The variable domains of the heavy chain and the light chain can be determined using any available method or numbering scheme and may include the variable domains as described, e.g., in WO 2018/207701, the contents of which are hereby incorporated by reference. In some embodiments, the variable domain of the heavy chain and/or the light chain may lack one or more amino acid residues on the carboxyl terminus of the variable domain (i.e., at the carboxyl terminus of the fourth framework domain) that may otherwise be included in descriptions of the variable domain based on certain numbering schemes. In some embodiments, the variable domain of the heavy chain and/or the light chain may include one or more amino acid residues on the carboxyl terminus of the variable domain (i.e., at the carboxyl terminus of the fourth framework domain) that may otherwise not be included in descriptions of the variable domain based on certain numbering schemes.

The term “hypervariable region,” “HVR,” or “HV,” when used herein refers to the regions of an antibody-variable domain that are hypervariable in sequence and/or form structurally defined loops. Generally, antibodies comprise six HVRs; three in the VH (H1, HZ, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al. Immunity 13:3745 (2000); Johnson and Wu in Methods in Molecular Biology 248:1-25 (Lo, ed., Human Press, Totowa, N.J., 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. The HVRs that are Kabat complementarity-determining regions (CDRs) are based on sequence variability and are the most commonly used (Kabat et al., Sequences of Proteins of Immunological Interest, 5th Ed. Public Health Service, National Institute of Health, Bethesda, Md. (1991)). Chothia HVRs refer instead to the location of the structural loops (Chothia and Lesk J. Mol. Biol. 196:901-917 (1987)). The “contact” HVRs are based on an analysis of the available complex crystal structures. The residues from each of these HVRs are noted below.

### Lop Kabat Chothia Contact

L1 L24-L34 L26-L34 L30-L36
L2 L50-L56 L50-L56 L46-L55
L3 L89-L97 L91-L96 L89-L96
H1 H31-H35B H26-H32 H30-H35B (Kabat Numbering)
H1 H31-H35 H26-H32 H30-H35 (Chothia Numbering)
H2 H50-H65 H53-H56 H47-H58
H3 H95-H102 H95-H102 H93-H101

Unless otherwise indicated, the variable-domain residues (HVR residues and framework region residues) are numbered according to Kabat et al., supra.

“Framework” or “FR” residues are those variable-domain residues other than the HVR residues as herein defined.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

An “acceptor human framework” for the purposes herein is a framework comprising the amino acid sequence of a VL or VH framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

An antibody that “binds to,” “specifically binds to” or is “specific for” a particular a polypeptide or an epitope on a particular polypeptide is one that binds to that particular polypeptide or epitope on a particular polypeptide without substantially binding to any other polypeptide or polypeptide epitope. In some embodiments, binding of an anti-CTLA4 binding protein described herein (e.g., anti-CTLA4 antibody or antigen-binding fragment thereof) to an unrelated, non-CTLA4 polypeptide is less than about 10% of the antibody binding to CTLA4 as measured by methods known in the art (e.g., enzyme-linked immunosorbent assay (ELISA)). In some embodiments, the binding protein (e.g., antibody) that binds to a CTLA4 (e.g., a murine CTLA4 and/or a human CTLA4) has an equilibrium dissociation constant (K_{D}) of ≤1 μM, ≤100 nM, ≤10 nM, ≤2 nM, ≤1 nM, ≤0.7 nM, ≤0.6 nM, ≤0.5 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M).

The term “CTLA4” or “CTLA4 protein” as provided herein includes any of the recombinant or naturally-occurring forms of the cytotoxic T-lymphocyte-associated protein 4 (CTLA4) or variants or homologs thereof that maintain CTLA4 protein activity (e.g. within at least 50%, 80%, 90%, 95%, 96%, 97%, 98%, 99% or 100% activity compared to CTLA4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CTLA4 polypeptide. In some embodiments, CTLA4 is the protein as identified by the NCBI sequence reference GI:83700231, homolog or functional fragment thereof. In some embodiments, CTLA4 is a human CTLA4. In some embodiments, CTLA4 is a murine CTLA4.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptors); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g., natural killer (NK) cells, neutrophils and macrophages) enable these cytotoxic effector cells to bind specifically to an antigen-bearing target cell and subsequently kill the target cell with cytotoxins. The antibodies “arm” the cytotoxic cells and are required for killing of the target cell by this mechanism. The primary cells for mediating ADCC, NK cells, express FcγRIII only, whereas monocytes express FcγRI, FcγRII and FcγRIII. Fc expression on hematopoietic cells is summarized in Table 3 on page 464 of Ravetch and Kinet, Annu. Rev. Immunol. 9: 457-92 (1991). In some embodiments, an anti-CTLA4 binding protein described herein (e.g., anti-CTLA4 antibody or antigen-binding fragment thereof) is engineered or expressed in cells that lack the ability to fucosylate the Fc glycan to have enhanced ADCC. To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and natural killer (NK) cells. Alternatively, or additionally, ADCC activity of the molecule of interest may be assessed in vivo, e.g., in an animal model such as that disclosed in Clynes et al., PNAS USA 95:652-656 (1998). Other Fc variants that alter ADCC activity and other antibody properties include those disclosed by Ghetie et al., Nat Biotech. 15:637-40, 1997; Duncan et al, Nature 332:563-564, 1988; Lund et al., J. Immunol 147:2657-2662, 1991; Lund et al, Mol Immunol 29:53-59, 1992; Alegre et al, Transplantation 57:1537-1543, 1994; Hutchins et al., Proc Natl. Acad Sci USA 92:11980-11984, 1995; Jefferis et al. Immunol Lett. 44:111-117, 1995; Lund et al., FASEB J9:115-119, 1995; Jefferis et al, Immunol Lett 54:101-104, 1996; Lund et al, J Immunol 157:4963-4969, 1996; Armour et al., Eur J Immunol 29:2613-2624, 1999; Idusogie et al, J Immunol 164:4178-4184, 200; Reddy et al, J Immunol 164:1925-1933, 2000; Xu et al., Cell Immunol 200:16-26, 2000; Idusogie et al, J Immunol 166:2571-2575, 2001; Shields et al., J Biol Chem 276:6591-6604, 2001; Jefferis et al. Immunol Lett 82:57-65, 2002; Presta et al., Biochem Soc Trans 30:487-490, 2002; Lazir et al., Proc. Natl. Acad. Sci. USA 103:4005-4010, 2006; U.S. Pat. Nos. 5,624,821; 5,885,573; 5,677,425; 6,165,745; 6,277,375; 5,869,046; 6,121,022; 5,624,821; 5,648,260; 6,194,551; 6,737,056; 6,821,505; 6,277,375; 7,335,742; and 7,317,091.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. Suitable native-sequence Fc regions for use in the antibodies of the invention include human IgG1, IgG2, IgG3 and IgG4.

“Binding affinity” as used herein refers to the strength of the non-covalent interactions between a single binding site of a molecule (e.g., an antibody) and its binding partner (e.g., an antigen). In some embodiments, the affinity of a binding protein (e.g., antibody) for a CTLA4 can generally be represented by an equilibrium dissociation constant (K_{D}). Affinity can be measured by common methods known in the art, including those described herein.

“Binding avidity” as used herein refers to the binding strength of multiple binding sites of a molecule (e.g., an antibody) and its binding partner (e.g., an antigen).

An “isolated” nucleic acid molecule encoding the antibodies herein is a nucleic acid molecule that is identified and separated from at least one contaminant nucleic acid molecule with which it is ordinarily associated in the environment in which it was produced. In some embodiments, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “pharmaceutical formulation” refers to a preparation that is in such form as to permit the biological activity of the active ingredient to be effective, and that contains no additional components that are unacceptably toxic to a subject to which the formulation would be administered. Such formulations are sterile.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids: antioxidants including ascorbic acid; low molecular weight (less than about 10 residues) polypeptide: proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA: sugar alcohols such as mannitol or sorbitol; salt-forming counterions such as sodium; and/or nonionic surfactants such as TWEEN™, polyethylene glycol (PEG), and PLURONICS™.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. An individual is successfully “treated”, for example, if one or more symptoms associated with a disorder (e.g., a neoplastic disease) are mitigated or eliminated. For example, an individual is successfully “treated” if treatment results in increasing the quality of life of those suffering from a disease, decreasing the dose of other medications required for treating the disease, reducing the frequency of recurrence of the disease, lessening severity of the disease, delaying the development or progression of the disease, and/or prolonging survival of individuals.

As used herein, “in conjunction with” or “in combination with” refers to administration of one treatment modality in addition to another treatment modality. As such, “in conjunction with” or “in combination with” refers to administration of one treatment modality before, during or after administration of the other treatment modality to the individual.

As used herein, the term “prevention” includes providing prophylaxis with respect to occurrence or recurrence of a disease in an individual. An individual may be predisposed to, susceptible to a disorder, or at risk of developing a disorder, but has not yet been diagnosed with the disorder. In some embodiments, anti-CTLA4 binding proteins (e.g., anti-CTLA4antibodies) described herein are used to delay development of a disorder.

As used herein, an individual “at risk” of developing a disorder may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of the disease, as known in the art. An individual having one or more of these risk factors has a higher probability of developing the disorder than an individual without one or more of these risk factors.

An “effective amount” refers to at least an amount effective, at dosages and for periods of time necessary, to achieve the desired or indicated effect, including a therapeutic or prophylactic result. An effective amount can be provided in one or more administrations. A “therapeutically effective amount” is at least the minimum concentration required to affect a measurable improvement of a particular disorder. A therapeutically effective amount herein may vary according to factors such as the disease state, age, sex, and weight of the patient, and the ability of the antibody to elicit a desired response in the individual. A therapeutically effective amount may also be one in which any toxic or detrimental effects of the antibody are outweighed by the therapeutically beneficial effects. A “prophylactically effective amount” refers to an amount effective, at the dosages and for periods of time necessary, to achieve the desired prophylactic result. Typically but not necessarily, since a prophylactic dose is used in subjects prior to or at the earlier stage of disease, the prophylactically effective amount can be less than the therapeutically effective amount.

“Chronic” administration refers to administration of the medicament(s) in a continuous as opposed to acute mode, so as to main the initial therapeutic effect (activity) for an extended period of time. “Intermittent” administration is treatment that is not consecutively done without interruption, but rather is cyclic in nature.

As used herein, an “individual” or a “subject” is a mammal. A “mammal” for purposes of treatment includes humans, domestic and farm animals, and zoo, sports, or pet animals, such as dogs, horses, rabbits, cattle, pigs, hamsters, gerbils, mice, ferrets, rats, cats, etc. In some embodiments, the individual or subject is human.

### I. Methods of Treatment

Provided herein are methods for treating or preventing a disease in a subject comprising administering to the subject an effective amount of an anti-CTLA4 binding protein described herein (e.g., anti-CTLA4 antibody or antigen-binding fragment thereof, e.g., Antibody B) or compositions thereof) and a PD-1 signaling agent (e.g., PD-1 or PD-L1 inhibitor). In some embodiments, the subject (e.g., a human patient) has been diagnosed with a neoplastic disorder (e.g., cancer) or is at risk of developing such a disorder.

For the prevention or treatment of disease, the appropriate dosage of an active agent, will depend on the type of disease to be treated, as defined above, the severity and course of the disease, whether the agent is administered for preventive or therapeutic purposes, previous therapy, the subject's clinical history and response to the agent, and the discretion of the attending physician. The agent is suitably administered to the subject at one time or over a series of treatments. In some embodiments of the methods described herein, an interval between administrations of an anti-CTLA4 binding protein (e.g., anti-CTLA4 antibody or antigen-binding fragment thereof) and a PD-1 signaling agent (e.g., PD-1 or PD-L1 inhibitor) is about one month or longer. In some embodiments, the interval between administrations is about two months, about three months, about four months, about five months, about six months or longer.

In some embodiments of the methods described herein, an interval between administrations of an anti-CTLA4 binding protein (e.g., anti-CTLA4 antibody or antigen-binding fragment thereof) and a PD-1 signaling agent (e.g., PD-1 or PD-L1 inhibitor) is about every 3 weeks. In some embodiments of the methods described herein, an interval between administrations of an anti-CTLA4 binding protein (e.g., anti-CTLA4 antibody or antigen-binding fragment thereof) and a PD-1 signaling agent (e.g., PD-1 or PD-L1 inhibitor) is weekly, every 2 weeks, every 3 weeks, or every 4 weeks. In some embodiments, the a PD-1 signaling agent (e.g., PD-1 or PD-L1 inhibitor) is administered every 3 weeks.

As used herein, an interval between administrations refers to the time period between one administration of the antibody and the next administration of the antibody. As used herein, an interval of about one month includes four weeks. In some embodiments, the interval between administrations is about two weeks, about three weeks, about four weeks, about eight weeks, about twelve weeks, about sixteen weeks, about twenty weeks, about twenty four weeks, or longer. In some embodiments, the treatment includes multiple administrations of the antibody, wherein the interval between administrations may vary. For example, the interval between the first administration and the second administration is about one month, and the intervals between the subsequent administrations are about three months. In some embodiments, the interval between the first administration and the second administration is about one month, the interval between the second administration and the third administration is about two months, and the intervals between the subsequent administrations are about three months. In some embodiments, an anti-CTLA4 binding protein (e.g., anti-CTLA4 antibody or antigen-binding fragment thereof) described herein is administered at a flat dose. Depending on the type and severity of the disease, about 1 μg/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. In some embodiments, an anti-CTLA4 binding protein (e.g., anti-CTLA4 antibody or antigen-binding fragment thereof) described herein is administered to a subject at a dosage from about 25 mg to about 500 mg per dose. In some embodiments, an anti-CTLA4 binding protein (e.g., anti-CTLA4 antibody or antigen-binding fragment thereof) described herein is administered to a subject at a dosage from about 0.1 mg/kg to about 10 mg/kg or about 1.0 mg/kg to about 10 mg/kg. In some embodiments, an anti-CTLA4 binding protein (e.g., anti-CTLA4 antibody or antigen-binding fragment thereof) described herein is administered to a subject at a dosage of about any of 0.1 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, or 10.0 mg/kg. In some embodiments, an anti-CTLA4 binding protein (e.g., anti-CTLA4 antibody or antigen-binding fragment thereof) described herein is administered to a subject at a dosage of between or between about 0.1 mg/kg and 10 mg/kg, between or between about 10 mg/kg and 20 mg/kg, between or between about 20 mg/kg and 30 mg/kg, between or between about 30 mg/kg and 40 mg/kg, between or between about 40 mg/kg and 50 mg/kg, between or between about 50 mg/kg and 60 mg/kg, between or between about 60 mg/kg and 70 mg/kg, or between or between about 70 mg/kg and 80 mg/kg. Any of the dosing frequency described above may be used.

A method of treatment contemplated herein is the treatment of a disorder or disease with an anti-CTLA4 binding protein (e.g., anti-CTLA4 antibody or antigen-binding fragment thereof) described herein and a PD-1 signaling agent (e.g., PD-1 or PD-L1 inhibitor). Disorders or diseases that are treatable with the formulations of this present invention include leukemia, lymphoma, head and neck cancer, colorectal cancer, prostate cancer, pancreatic cancer, melanoma, breast cancer, neuroblastoma, lung cancer, ovarian cancer, osteosarcoma, bladder cancer, cervical cancer, liver cancer, kidney cancer, skin cancer (e.g., Merkel cell carcinoma) or testicular cancer.

In some embodiments, provided herein is a method of treatment or prevention of a cancer by administration of an anti-CTLA4 binding protein (e.g., anti-CTLA4 antibody or antigen-binding fragment thereof) and a PD-1 signaling agent (e.g., PD-1 or PD-L1 inhibitor) described herein. As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with an anti-CTLA4 binding protein (e.g., anti-CTLA4 antibody or antigen-binding fragment thereof) and a PD-1 signaling agent (e.g., PD-1 or PD-L1 inhibitor), pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant. Herceptin resistant, HER2 positive, doxorubicin resistant, tamoxifen resistant, ductal carcinoma, lobular carcinoma, primary, metastatic), ovarian cancer, pancreatic cancer, liver cancer (e.g. hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

In some embodiments, provided herein is a method of treatment or prevention of a leukemia by administration of an anti-CTLA4 binding protein (e.g., anti-CTLA4 antibody or antigen-binding fragment thereof) and a PD-1 signaling agent (e.g., PD-1 or PD-L1 inhibitor) described herein. The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

In some embodiments, provided herein is a method of treatment or prevention of a sarcoma by administration of an anti-CTLA4 binding protein (e.g., anti-CTLA4 antibody or antigen-binding fragment thereof) and a PD-1 signaling agent (e.g., PD-1 or PD-L1 inhibitor) described herein. The term “sarcoma” generally refers to a tumor which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

In some embodiments, provided herein is a method of treatment or prevention of a melanoma by administration of an anti-CTLA4 binding protein (e.g., anti-CTLA4 antibody or antigen-binding fragment thereof) and a PD-1 signaling agent (e.g., PD-1 or PD-L1 inhibitor) described herein. The term “melanoma” is taken to mean a tumor arising from the melanocytic system of the skin and other organs. Melanomas that may be treated with a compound, pharmaceutical composition, or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

In some embodiments, provided herein is a method of treatment or prevention of a carcinoma by administration of an anti-CTLA4 binding protein (e.g., anti-CTLA4 antibody or antigen-binding fragment thereof) and a PD-1 signaling agent (e.g., PD-1 or PD-L1 inhibitor) described herein. The term “carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangioccllular carcinoma, chorionic carcinoma, colloid carcinoma, comcdo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatinifomi carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthie cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

In some embodiments, provided herein is a method of treatment or prevention of metastatic cancer by administration of an anti-CTLA4 binding protein (e.g., anti-CTLA4 antibody or antigen-binding fragment thereof) and a PD-1 signaling agent (e.g., PD-1 or PD-L1 inhibitor) described herein. As used herein, the terms “metastasis,” “metastatic,” and “metastatic cancer” can be used interchangeably and refer to the spread of a neoplastic disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

In some embodiments, diseases or disorders that may benefit by CTLA4 binding proteins (e.g., Antibody B) in combination with a PD-1 signaling agent (e.g., PD-1 or PD-L1 inhibitor) described herein include a disease (e.g., diabetes, cancer (e.g. prostate cancer, renal cancer, metastatic cancer, melanoma, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma)) caused by (in whole or in part), or a symptom of the disease is caused by (in whole or in part) CTLA4 or CTLA4 activity or function and/or PD-1 signaling activity or function.

Described herein are methods of treating disorders in a subject (e.g., disorders that benefit from administration of an anti-PD-1 therapy). For example, an anti-PD-1 therapy described herein can be administered e.g., as a combination therapy with a CTLA-4 antibody, for a period sufficient to achieve clinical benefit or according to a regimen as determined by a physician (e.g., an anti-PD-1 therapy is administered in dosage amounts and number of treatment cycles as determined by a physician).

In embodiments, methods described herein are useful for treating T-cell dysfunctional disorders (e.g., cancer). In embodiments, methods described herein are useful for reducing tumors or inhibiting the growth of tumor cells in a subject.

In embodiments, methods described herein are useful for increasing T cell activation or T cell effector function in a subject.

In embodiments, methods described herein are useful for inducing an immune response in a subject.

In embodiments, methods described herein are useful for enhancing an immune response or increasing the activity of an immune cell in a subject.

The inventive methods can be used to treat any type of autoimmune disease (i.e., as disease or disorder caused by immune system over-activity in which the body attacks and damages its own tissues), such as those described in, for example, MacKay I. R, and Rose N. R., eds., The Autoimmune Diseases, Fifth Edition, Academic Press, Waltham, Mass. (2014). Examples of autoimmune diseases that can be treated by the inventive method include, but are not limited to, multiple sclerosis, type 1 diabetes mellitus, rheumatoid arthritis, scleroderma, Crohn's disease, psoriasis, systemic lupus erythematosus (SLE), and ulcerative colitis. When the inventive method treats an autoimmune disease, a PD-1 antibody agent can be used in combination with an anti-inflammatory agent including, for example, corticosteroids (e.g., prednisone and fluticasone) and non-steroidal anti-inflammatory drugs (NSAIDs) (e.g., aspirin, ibuprofen, and naproxen).

PD-1 is abnormally expressed in a variety of cancers (see, e.g., Brown at al, J. Immunol., 170: 1257-1266 (2003); and Flies et. al, Yale Journal of Biology and Medicine, 84: 409-421 (2011)), and PD-L1 expression in some renal cell carcinoma patients correlates with tumor aggressiveness. The inventive methods can be used to treat any type of cancer known in the art.

In embodiments, a cancer that is adenocarcinoma, adenocarcinoma of the lung, acute myeloid leukemia (“AML”), acute lymphoblastic leukemia (“ALL”), adrenocortical carcinoma, anal cancer, appendiceal cancer, B-cell derived leukemia, B-cell derived lymphoma, bladder cancer, brain cancer, breast cancer (e.g., triple negative breast cancer (TNBC)), cancer of the fallopian tube(s), cancer of the testes, cerebral cancer, cervical cancer, choriocarcinoma, chronic myelogenous leukemia, a CNS tumor, colon adenocarcinoma, colon cancer, colorectal cancer, diffuse intrinsic pontine glioma (DIPG), diffuse large B cell lymphoma (“DLBCL”), embryonal rhabdomyosarcoma (ERMS), endometrial cancer, epithelial cancer, esophageal cancer, Ewing's sarcoma, follicular lymphoma (“FL”), gall bladder cancer, gastric cancer, gastrointestinal cancer, glioma, head and neck cancer, a hematological cancer, hepatocellular cancer, Hodgkin's lymphoma/primary mediastinal B-cell lymphoma, kidney cancer, kidney clear cell cancer, laryngeal cancer, leukemia, liver cancer, lung cancer, lymphoma, melanoma, Merkel cell carcinoma, mesothelioma, monocytic leukemia, multiple myeloma, myeloma, a neuroblastic-derived CNS tumor, non-Hodgkin's lymphoma (NHL), non-small cell lung cancer (NSCLC), oral cancer, osteosarcoma, ovarian cancer, ovarian carcinoma, pancreatic cancer, peritoneal cancer, primary peritoneal cancer, prostate cancer, relapsed or refractory classic Hodgkin's Lymphoma (cHL), renal cell carcinoma, rectal cancer, salivary gland cancer (e.g., a salivary gland tumor), sarcoma, skin cancer, small cell lung cancer, small intestine cancer, squamous cell carcinoma of the anogenital region (e.g., squamous cell carcinoma of the anus, penis, cervix, vagina, or vulva), squamous cell carcinoma of the esophagus, squamous cell carcinoma of the head and neck (SCHNC), squamous cell carcinoma of the lung, stomach cancer, T-cell derived leukemia, T-cell derived lymphoma, thymic cancer, a thymoma, thyroid cancer, uveal melanoma, urothelial cell carcinoma, uterine cancer, uterine endometrial cancer, uterine sarcoma, vaginal cancer, vulvar cancer, or Wilms tumor.

In other embodiments, a cancer is a head and neck cancer, a lung cancer (e.g., a non-small cell lung cancer (NSCLC)), a renal cancer, a bladder cancer, a melanoma, Merkel cell carcinoma (see, e.g., Bhatia et al., Curr. Oncol. Rep., 13(6): 488-497 (2011), a cervical cancer, a vaginal cancer, a vulvar cancer, a uterine cancer, a endometrial cancer, an ovarian cancer, a fallopian tube cancer, a breast cancer, a prostate cancer, a salivary gland tumor, a thymoma, a adrenocortical carcinoma, a esophageal cancer, a gastric cancer, a colorectal cancer, an appendiceal cancer, a urothelial cell carcinoma, or a squamous cell carcinoma (e.g., of the lung; of the anogenital region including anus, penis, cervix, vagina, or vulva; or of the esophagus). In some embodiments, a cancer for treatment in the context of the present disclosure is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma.

In some embodiments, a patient or population of patients have a hematological cancer. In some embodiments, the patient has a hematological cancer such as Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), acute lymphoblastic leukemia (“ALL”), or Multiple myeloma (“MM”). In embodiments, a cancer is a blood-borne cancer such as acute lymphoblastic leukemia(“ALL”), acute lymphoblastic B-cell leukemia, acute lymphoblastic T-cell leukemia, acute myeloblastic leukemia (“AML”), acute promyelocytic leukemia(“APL”), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia(“CML”), chronic lymphocytic leukemia(“CLL”), hairy cell leukemia and multiple myeloma; acute and chronic leukemias such as lymphoblastic, myelogenous, lymphocytic, and myelocytic leukemias.

In embodiments a cancer is a lymphoma such as Hodgkin's disease, non-Hodgkin's Lymphoma, Multiple mycloma, Waldenstrom's macroglobulinemia, Heavy chain disease and Polycythemia vera.

In embodiments, a cancer is a squamous cell carcinoma. In embodiments, a cancer is squamous cell carcinoma of the lung. In embodiments, a cancer is squamous cell carcinoma of the esophagus. In embodiments, a cancer is head and neck squamous cell carcinoma (HNSCC).

In embodiments, a cancer is squamous cell carcinoma of the anogenital region (e.g., of the anus, penis, cervix, vagina, or vulva).

In embodiments, a cancer is bladder cancer, breast cancer (e.g., triple negative breast cancer (TNBC)), cancer of the fallopian tube(s), cholangiocarcinoma, colon adenocarcinoma, endometrial cancer, esophageal cancer, Ewing's sarcoma, gastric cancer, kidney clear cell cancer, lung cancer (e.g., lung adenocarcinoma or lung squamous cell cancer), mesothelioma, ovarian cancer, pancreatic cancer, peritoneal cancer, prostate cancer, uterine endometrial cancer, or uveal melanoma. In embodiments, a cancer is ovarian cancer, cancer of the fallopian tube(s), or peritoneal cancer. In embodiments, a cancer is breast cancer (e.g., TNBC). In embodiments, a cancer is lung cancer (e.g., non-small cell lung cancer). In embodiments, a cancer is prostate cancer.

In embodiments, a cancer is a CNS or brain cancer such as neuroblastoma (NB), glioma, diffuse intrinsic pontine glioma (DIPG), pilocytic astrocytoma, astrocytoma, anaplastic astrocytoma, glioblastoma multiforme, medulloblastoma, craniopharyngioma, ependymoma, pinealoma, hemangioblastoma, acoustic neuroma, oligodendroglioma, meningioma, vestibular schwannoma, adenoma, metastatic brain tumor, meningioma, spinal tumor, or medulloblastoma. In embodiments, a cancer is a CNS tumor.

In some embodiments, a patient or population of patients have a solid tumor. In embodiments, a cancer is a solid tumor such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, osteosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, uterine cancer, testicular cancer, non small cell lung cancer (NSCLC), small cell lung carcinoma, bladder carcinoma, lung cancer, epithelial carcinoma, skin cancer, melanoma, neuroblastoma (NB), or retinoblastoma. In some embodiments, the tumor is an advanced stage solid tumor. In some embodiments, the tumor is a metastatic solid tumor. In some embodiments, the patient has a MSI-H solid tumor.

In some embodiments, a patient or population of patients to be treated by the methods of the present invention have or are susceptible to cancer, such as a head and neck cancer, a lung cancer (e.g., a non-small cell lung cancer (NSCLC)), a renal cancer, a bladder cancer, a melanoma, Merkel cell carcinoma, a cervical cancer, a vaginal cancer, a vulvar cancer, a uterine cancer, a endometrial cancer, an ovarian cancer, a fallopian tube cancer, a breast cancer, a prostate cancer, a salivary gland tumor, a thymoma, a adrenocortical carcinoma, a esophageal cancer, a gastric cancer, a colorectal cancer, an appendiceal cancer, a urothelial cell carcinoma, or a squamous cell carcinoma (e.g., of the lung, of the anogenital region including anus, penis, cervix, vagina, or vulva; or of the esophagus). In some embodiments, a patient or population of patients to be treated by the methods of the present invention have or are susceptible to lung cancer (e.g., NSCLC), renal cancer, melanoma, cervical cancer, colorectal cancer, or endometrial cancer (e.g., MSS endometrial cancer or MSI-H endometrial cancer).

In some embodiments, a cancer is a gynecologic cancer (i.e., a cancer of the female reproductive system such as ovarian cancer, fallopian tube cancer, cervical cancer, vaginal cancer, vulvar cancer, uterine cancer, or primary peritoneal cancer, or breast cancer). In some embodiments, cancers of the female reproductive system include, but are not limited to, ovarian cancer, cancer of the fallopian tube(s), peritoneal cancer, and breast cancer.

In embodiments, a cancer is ovarian cancer (e.g., serous or clear cell ovarian cancer). In embodiments, a cancer is fallopian tube cancer (e.g., serous or clear cell fallopian tube cancer). In embodiments, a cancer is primary peritoneal cancer (e.g., serous or clear cell primary peritoneal cancer).

In some embodiments, an ovarian cancer is an epithelial carcinoma. Epithelial carcinomas make up 85% to 90% of ovarian cancers. While historically considered to start on the surface of the ovary, new evidence suggests at least some ovarian cancer begins in special cells in a part of the fallopian tube. The fallopian tubes are small ducts that link a woman's ovaries to her uterus that are a part of a woman's reproductive system. In a normal female reproductive system, there are two fallopian tubes, one located on each side of the uterus. Cancer cells that begin in the fallopian tube may go to the surface of the ovary early on. The term ‘ovarian cancer’ is often used to describe epithelial cancers that begin in the ovary, in the fallopian tube, and from the lining of the abdominal cavity, call the peritoneum. In some embodiments, the cancer is or comprises a germ cell tumor. Germ cell tumors are a type of ovarian cancer develops in the egg-producing cells of the ovaries. In some embodiments, a cancer is or comprises a stromal tumor. Stromal tumors develop in the connective tissue cells that hold the ovaries together, which sometimes is the tissue that makes female hormones called estrogen. In some embodiments, a cancer is or comprises a granulosa cell tumor. Granulosa cell tumors may secrete estrogen resulting in unusual vaginal bleeding at the time of diagnosis. In some embodiments, a gynecologic cancer is associated with homologous recombination repair deficiency/homologous repair deficiency (“HRD”) and/or BRCA 1/2 mutation(s). In some embodiments, a gynecologic cancer is platinum-sensitive. In some embodiments, a gynecologic cancer has responded to a platinum-based therapy. In some embodiments, a gynecologic cancer has developed resistance to a platinum-based therapy. In some embodiments, a gynecologic cancer has at one time shown a partial or complete response to platinum-based therapy (e.g., a partial or complete response to the last platinum-based therapy or to the penultimate platinum-based therapy). In some embodiments, a gynecologic cancer is now resistant to platinum-based therapy.

In embodiments, a cancer is a breast cancer. Usually breast cancer either begins in the cells of the milk producing glands, known as the lobules, or in the ducts. Less commonly breast cancer can begin in the stromal tissues. These include the fatty and fibrous connective tissues of the breast. Over time the breast cancer cells can invade nearby tissues such the underarm lymph nodes or the lungs in a process known as metastasis. The stage of a breast cancer, the size of the tumor and its rate of growth are all factors which determine the type of treatment that is offered. Treatment options include surgery to remove the tumor, drug treatment which includes chemotherapy and hormonal therapy, radiation therapy and immunotherapy. The prognosis and survival rate varies widely; the five year relative survival rates vary from 98% to 23% depending on the type of breast cancer that occurs. Breast cancer is the second most common cancer in the world with approximately 1.7 million new cases in 2012 and the fifth most common cause of death from cancer, with approximately 521,000 deaths. Of these cases, approximately 15% are triple-negative, which do not express the estrogen receptor, progesterone receptor (PR) or HER2. In some embodiments, triple negative breast cancer (TNBC) is characterized as breast cancer cells that are estrogen receptor expression negative (<1% of cells), progesterone receptor expression negative (<1% of cells), and HER2-negative.

In embodiments, a cancer is ER-positive breast cancer, ER-negative breast cancer, PR-positive breast cancer, PR-negative breast cancer, HER2-positive breast cancer, HER2-negative breast cancer, BRCA1/2-positive breast cancer, BRCA1/2-negative cancer, or triple negative breast cancer (TNBC). In embodiments, a cancer is triple negative breast cancer (TNBC).

In some embodiments, a breast cancer is a metastatic breast cancer. In some embodiments, a breast cancer is an advanced breast cancer. In some embodiments, a cancer is a stage 11, stage III or stage IV breast cancer. In some embodiments, a cancer is a stage IV breast cancer. In some embodiments, a breast cancer is a triple negative breast cancer.

In some embodiments, a patient or a population of patients to be treated by the methods of the present disclosure have or are susceptible to endometrial cancer (“EC”). Endometrial carcinoma is the most common cancer of the female genital, tract accounting for 10-20 per 100,000 person-years. The annual number of new cases of endometrial cancer (EC) is estimated at about 325 thousand worldwide. Further, EC is the most commonly occurring cancer in post-menopausal women. About 53% of endometrial cancer cases occur in developed countries. In 2015, approximately 55,000 cases of EC were diagnosed in the U.S. and no targeted therapies are currently approved for use in EC. There is a need for agents and regimens that improve survival for advanced and recurrent EC in 1 L and 2 L settings. Approximately 10,170 people are predicted to die from EC in the U.S. in 2016. The most common histologic form is endometrioid adenocarcinoma, representing about 75-80% of diagnosed cases. Other histologic forms include uterine papillary serous (less than 10%), clear cell 4%, mucinous 1%, squamous less than 1% and mixed about 10%.

From the pathogenetic point of view, EC falls into two different types, so-called types I and II. Type I tumors are low-grade and estrogen-related endometrioid carcinomas (EEC) while type II are non-endometrioid (NEEC) (mainly serous and clear cell) carcinomas. The World Health Organization has recently updated the pathologic classification of EC, recognizing nine different subtypes of EC, but EEC and serous carcinoma (SC) account for the vast majority of cases. EECs are estrogen-related carcinomas, which occur in perimenopausal patients, and are preceded by precursor lesions (endometrial hyperplasia/endometrioid intraepithelial neoplasia). Microscopically, lowgrade EEC (EEC 1-2) contains tubular glands, somewhat resembling the proliferative endometrium, with architectural complexity with fusion of the glands and cribriform pattern. High-grade EEC shows solid pattern of growth. In contrast, SC occurs in postmenopausal patients in absence of hyperestrogenism. At the microscope, SC shows thick, fibrotic or edematous papillae with prominent stratification of tumor cells, cellular budding, and anaplastic cells with large, eosinophilic cytoplasms. The vast majority of EEC are low grade tumors (grades 1 and 2), and are associated with good prognosis when they are restricted to the uterus. Grade 3 EEC (EEC3) is an aggressive tumor, with increased frequency of lymph node metastasis. SCs are very aggressive, unrelated to estrogen stimulation, mainly occurring in older women. EEC 3 and SC are considered high-grade tumors. SC and EEC3 have been compared using the surveillance, epidemiology and End Results (SEER) program data from 1988 to 2001. They represented 10% and 15% of EC respectively, but accounted for 39% and 27% of cancer death respectively.

Endometrial cancers can also be classified into four molecular subgroups: (1) ultramutated/POLE-mutant; (2) hypermutated MSI+(e.g., MSI-H or MSI-L); (3) copy number low/microsatellite stable (MSS); and (4) copy number high/serous-like. Approximately 28% of cases are MSI-high. (Murali, Lancet Oncol. (2014). In some embodiments, a patient has a mismatch repair deficient subset of 2 L endometrial cancer.

In embodiments, an endometrial cancer is metastatic endometrial cancer.

In embodiments, a patient has a MSS endometrial cancer.

In embodiments, a patient has a MSI-H endometrial cancer.

In embodiments, a cancer is a lung cancer. In embodiments, a lung cancer is a squamous cell carcinoma of the lung. In embodiments, a lung cancer is small cell lung cancer (SCLC). In embodiments, a lung cancer is non-small cell lung cancer (NSCLC) such as squamous NSCLC. In embodiments, a lung cancer is an ALK-translocated lung cancer (e.g., ALK-translocated NSCLC). In embodiments, a lung cancer is an EGFR-mutant lung cancer (e.g., EGFR-mutant NSCLC).

In embodiments, a cancer is a colorectal (CRC) cancer (e.g., a solid tumor). In embodiments, a colorectal cancer is an advanced colorectal cancer. In embodiments, a colorectal cancer is a metastatic colorectal cancer. In embodiments, a colorectal cancer is a MSI-H colorectal cancer. In embodiments, a colorectal cancer is a MSS colorectal cancer. In embodiments, a colorectal cancer is a POLE-mutant colorectal cancer. In embodiments, a colorectal cancer is a POLD-mutant colorectal cancer. In embodiments, a colorectal cancer is a high TMB colorectal cancer.

In embodiments, a cancer is a melanoma. In embodiments, a melanoma is an advanced melanoma. In embodiments, a melanoma is a metastatic melanoma. In embodiments, a melanoma is a MSI-H melanoma. In embodiments, a melanoma is a MSS melanoma. In embodiments, a melanoma is a POLE-mutant melanoma. In embodiments, a melanoma is a POLD-mutant melanoma. In embodiments, a melanoma is a high TMB melanoma.

In embodiments, a cancer is an advanced cancer.

In embodiments, a cancer is a metastatic cancer.

In embodiments, a cancer is a recurrent cancer (e.g., a recurrent gynecological cancer such as recurrent epithelial ovarian cancer, recurrent fallopian tube cancer, recurrent primary peritoneal cancer, or recurrent endometrial cancer).

Cancers that can be treated with methods described herein include cancers associated with a high tumor mutation burden (TMB), cancers that microsatellite stable (MSS), cancers that are characterized by microsatellite instability, cancers that have a high microsatellite instability status (MSI-H), cancers that have low microsatellite instability status (MSI-L), cancers associated with high TMB and MSI-H (e.g., cancers associated with high TMB and MSI-L or MSS), cancers having a defective DNA mismatch repair system, cancers having a defect in a DNA mismatch repair gene, hypermutated cancers, cancers having homologous recombination repair deficiency/homologous repair deficiency (“HRD”), cancers comprising a mutation in polymerase delta (POLD), and cancers comprising a mutation in polymerase epsilon (POLE).

In some embodiments, a tumor to be treated is characterized by microsatellite instability. In some embodiments, a tumor is characterized by microsatellite instability high status (MSI-H). Microsatellite instability (“MSI”) is or comprises a change that in the DNA of certain cells (such as tumor cells) in which the number of repeats of microsatellites (short, repeated sequences of DNA) is different than the number of repeats that was contained in the DNA from which it was inherited. About 15% of sporadic colorectal cancers (CRC) harbor widespread alterations in the length of microsatellite (MS) sequences, known as microsatellite instability (MSI) (Boland and Goel, 2010). Sporadic MSI CRC tumors display unique clinicopathological features including near-diploid karyotype, higher frequency in older populations and in females, and a better prognosis (de la Chapelle and Hampel, 2010; Popat et al., 2005). MSI is also present in other tumors, such as in endometrial cancer (EC) of the uterus, the most common gynecological malignancy (Duggan et al., 1994). The same reference Bethesda panel originally developed to screen an inherited genetic disorder (Lynch syndrome) (Umar et al., 2004) is currently applied to test MSI for CRCs and ECs. However, the genes frequently targeted by MSI in CRC genomes rarely harbor DNA slippage events in EC genomes (Gurin et al., 1999).

Microsatellite instability arises from a failure to repair replication-associated errors due to a defective DNA mismatch repair (MMR) system. This failure allows persistence of mismatch mutations all over the genome, but especially in regions of repetitive DNA known as microsatellites, leading to increased mutational load. It has been demonstrated that at least some tumors characterized by MSI-H have improved responses to certain anti-PD-1 agents (Le et al., (2015) N. Engl. J. Med. 372(26):2509-2520; Westdorp et al., (2016) Cancer Immunol. Immunother. 65(10):1249-1259). In some embodiments, a cancer has a microsatellite instability of high microsatellite instability (e.g., MSI-H status). In some embodiments, a cancer has a microsatellite instability status of low microsatellite instability (e.g., MSI-Low). In some embodiments, a cancer has a microsatellite instability status of microsatellite stable (e.g., MSS status). In some embodiments microsatellite instability status is assessed by a next generation sequencing (NGS)-based assay, an immunohistochemistry (IHC)-based assay, and/or a PCR-based assay. In some embodiments, microsatellite instability is detected by NGS. In some embodiments, microsatellite instability is detected by IHC. In some embodiments, microsatellite instability is detected by PCR.

In embodiments, a patient has a MSI-L cancer.

In embodiments, a patient has a MSI-H cancer. In some embodiments, a patient has a MSI-H solid tumor. In embodiments, a MSI-H cancer is MSI-H endometrial cancer. In embodiments, a MSI-H cancer is a solid tumor. In embodiments, a MSI-H cancer is a metastatic tumor. In embodiments, a MSI-H cancer is endometrial cancer. In embodiments, a MSI-H cancer is a non-endometrial cancer. In embodiments, a MSI-H cancer is colorectal cancer.

In embodiments, a patient has a MSS cancer. In embodiments, a MSS cancer is MSS endometrial cancer.

In embodiments, a cancer is associated with a POLE (DNA polymerase epsilon) mutation (i.e., a cancer is a POLE-mutant cancer). In embodiments, a POLE mutation is a mutation in the exonuclease domain. In embodiments, a POLE mutation is a germline mutation. In embodiments, a POLE mutation is a sporadic mutation. In embodiments, a MSI cancer also is associated with a POLE mutation. In embodiments, a MSS cancer also is associated with a POLE mutation. In embodiments, a POLE mutation is identified using sequencing. In embodiments, a POLE-mutant cancer is endometrial cancer. In embodiments, a POLE-mutant cancer is colon cancer. In embodiments, a POLE-mutant cancer is pancreatic cancer, ovarian cancer, or cancer of the small intestine.

In embodiments, a cancer is associated with a POLD (DNA polymerase delta) mutation (i.e., a cancer is a POLD-mutant cancer). In embodiments, a POLD mutation is a mutation in the exonuclease domain. In embodiments, a POLD mutation is a somatic mutation. In embodiments, a POLD mutation is a germline mutation. In embodiments, a POLD-mutant cancer is identified using sequencing. In embodiments, a POLD-mutant cancer is endometrial cancer. In embodiments, a POLD-mutant cancer is colorectal cancer. In embodiments, a POLD-mutant cancer is brain cancer.

In some embodiments, a patient has a mismatch repair deficient (MMRd) cancer.

In embodiments, a MMRd cancer is colorectal cancer.

Microsatellite instability may arise from a failure to repair replication-associated errors due to a defective DNA mismatch repair (MMR) system. This failure allows persistence of mismatch mutations all over the genome, but especially in regions of repetitive DNA known as microsatellites, leading to increased mutational load that may improve responses to certain anti-PD-1 agents. Id. In some embodiments MSI-H status is assess by a NGS-based assay and/or a PCR-based MSI assay. In some embodiments, microsatellite instability is detected by next generation sequencing. In embodiments, microsatellite instability is detected using immunohistochemistry (IHC) testing.

In embodiments, a cancer (e.g., a MMRd cancer) is characterized by a high tumor mutation burden (i.e., a cancer is a high TMB cancer). In some embodiments, the cancer is associated with high TMB and MSI-H. In some embodiments, the cancer is associated with high TMB and MSI-L or MSS. In some embodiments, the cancer is endometrial cancer associated with high TMB. In some related embodiments, the endometrial cancer is associated with high TMB and MSI-H. In some related embodiments, the endometrial cancer is associated with high TMB and MSI-L or MSS. In embodiments, a high TMB cancer is colorectal cancer. In embodiments, a high TMB cancer is lung cancer (e.g., small cell lung cancer (SCLC) or non-small cell lung cancer (NSCLC) such as squamous NSCLC or non-squamous NSCLC). In embodiments, a high TMB cancer is melanoma. In embodiments, a high TMB cancer is urothelial cancer.

In embodiments, a patient has a cancer with elevated expression of tumor-infiltrating lymphocytes (TILs), i.e., a patient has a high-TIL cancer. In embodiments, a high-TIL cancer is breast cancer (e.g., triple negative breast cancer (TNBC) or HER2-positive breast cancer). In embodiments, a high-TIL cancer is a metastatic cancer (e.g., a metastatic breast cancer).

In embodiments, immune-related gene expression signatures can be predictive of a response to an anti-PD-1 therapy for cancer as described herein. For example, a gene panel that includes genes associated with IFN-γ signaling can be useful in identifying cancer patients who would benefit from anti-PD-1 therapy. Exemplary gene panels are described in Ayers et al., J. Clin. Invest., 127(8):2930-2940, 2017. In embodiments, a cancer patient has a cancer that is breast cancer (e.g., TNBC) or ovarian cancer. In embodiments, a cancer patient has a cancer that is bladder cancer, gastric cancer, biliary cancer, esophageal cancer, or head and neck squamous cell carcinoma (HNSCC). In embodiments, a cancer patient has a cancer that is anal cancer or colorectal cancer.

In some embodiments, a patient has a tumor that expresses PD-L1. In some embodiments, PD-L1 status is evaluated in a patient or patient population. In some embodiments, mutational load and baseline gene expression profiles in archival or fresh pre-treatment biopsies are evaluated before, during and/or after treatment with an anti-PD-1 antibody agent. In some embodiments, the status and/or expression of TIM-3 and/or LAG-3 are evaluated in patients.

In some embodiments, at least some of the patients in the cancer patient population have not previously been treated with one or more different cancer treatment modalities.

In some embodiments, a patient has previously been treated with one or more different cancer treatment modalities (e.g., one or more of surgery, radiotherapy, chemotherapy or immunotherapy). In embodiments, a subject has previously been treated with two or more different cancer treatment modalities (e.g., one or more of surgery, radiotherapy, chemotherapy, or immunotherapy). In embodiments, a subject has been previously treated with a cytotoxic therapy. In embodiments, a subject has been previously treated with chemotherapy. In embodiments, a subject has previously been treated with two different cancer treatment modalities (e.g., one or more of surgery, radiotherapy, chemotherapy, or immunotherapy). In embodiments, a subject has previously been treated with three different cancer treatment modalities (e.g., one or more of surgery, radiotherapy, chemotherapy, or immunotherapy).

In embodiments of methods described herein, a method further comprises administering one or more of surgery, a radiotherapy, a chemotherapy, an immunotherapy, an anti-angiogenic agent, or an anti-inflammatory. In embodiments, a method further comprises administering a chemotherapy.

In some embodiments, at least some of the patients in the cancer patient population have previously been treated with chemotherapy (e.g., platinum-based chemotherapy). For example, a patient who has received two lines of cancer treatment can be identified as a 2 L cancer patient (e.g., a 2 L NSCLC patient). In embodiments, a patient has received two lines or more lines of cancer treatment (e.g., a 2 L+ cancer patient such as a 2 L+ endometrial cancer patient). In embodiments, a patient has not been previously treated with an anti-PD-1 therapy. In embodiments, a patient previously received at least one line of cancer treatment (e.g., a patient previously received at least one line or at least two lines of cancer treatment). In embodiments, a patient previously received at least one line of treatment for metastatic cancer (e.g., a patient previously received one or two lines of treatment for metastatic cancer).

In embodiments, a subject is resistant to treatment with an agent that inhibits PD-1.

In embodiments, a subject is refractory to treatment with an agent that inhibits PD-1.

In embodiments, a method described herein sensitizes the subject to treatment with an agent that inhibits PD-1.

In embodiments, a subject comprises an exhausted immune cell (e.g., an exhausted immune cell that is an exhausted T cell).

In embodiments of methods described herein, a subject is an animal (e.g., a mammal). In embodiments, a subject is a human. In embodiments, a subject is a non-human mammal (e.g., mice, rats, rabbits, or non-human primates). Accordingly, methods described herein can be useful in both treatment of humans and in veterinary medicine.

In embodiments, a PD-1 inhibitor (e.g., an anti-PD-1 antibody) is administered intravenously (e.g., by intravenous infusion).

### Measuring Tumor Response

In some embodiments, a clinical benefit is a complete response (“CR”), a partial response (“PR”) or a stable disease (“SD”). In some embodiments, a clinical benefit corresponds to at least SD. In some embodiments, a clinical benefit corresponds to at least a PR. In some embodiments, a clinical benefit corresponds to a CR. In some embodiments, at least 1%, 2%, 3%, 4%, 5%, 6%, 7%, 8%, 9%, 10%, 15%, 20%, 25%, 30%, 35%, 40%, 50%, 55%, 60%, 65%, 70%, 75%, 80%, 85%, 90% or 95% of patients achieve a clinical benefit. In some embodiments, at least 5% of patients achieve a clinical benefit. In some embodiments, at least 5% of patients achieve SD. In some embodiments, at least 5% of patients achieve at least a PR. In some embodiments, at least 5% of patients achieve CR. In some embodiments, at least 20% of patients achieve a clinical benefit. In some embodiments, at least 20% of patients achieve SD.

In some embodiments, the clinical benefit (e.g., SD, PR and/or CR) is determined in accordance with Response Evaluation Criteria in Solid Tumors (RECIST). In some embodiments, the clinical benefit (e.g., SD, PR and/or CR) is determined in accordance RECIST guidelines.

In some embodiments, tumor response can be measured by, for example, the RECIST v 1.1 guidelines. The guidelines are provided by E. A. Eisenhauer, et al., “New response evaluation criteria in solid tumors: Revised RECIST guideline (version 1.1.),” Eur. J. of Cancer, 45: 228-247 (2009), which is incorporated by reference in its entirety. In some embodiments, RECIST guidelines may serve as a basis for all protocol guidelines related to disease status. In some embodiments, RECIST guidelines are used to assess tumor response to treatment and/or date of disease progression.

RECIST guidelines require, first, estimation of the overall tumor burden at baseline, which is used as a comparator for subsequent measurements. Tumors can be measured via use of any imaging system known in the art, for example, by a CT scan, or an X-ray. Measurable disease is defined by the presence of at least one measurable lesion. In studies where the primary endpoint is tumor progression (either time to progression or proportion with progression at a fixed date), the protocol must specify if entry is restricted to those with measurable disease or whether patients having non-measurable disease only are also eligible.

When more than one measurable lesion is present at baseline, all lesions up to a maximum of five lesions total (and a maximum of two lesions per organ) representative of all involved organs should be identified as target lesions and will be recorded and measured at baseline (this means in instances where patients have only one or two organ sites involved a maximum of two and four lesions respectively will be recorded).

Target lesions should be selected on the basis of their size (lesions with the longest diameter), be representative of all involved organs, but in addition should be those that lend themselves to reproducible repeated measurements.

Lymph nodes merit special mention since they are normal anatomical structures which may be visible by imaging even if not involved by tumor. Pathological nodes which are defined as measurable and may be identified as target lesions must meet the criterion of a short axis of P15 mm by CT scan. Only the short axis of these nodes will contribute to the baseline sum. The short axis of the node is the diameter normally used by radiologists to judge if a node is involved by solid tumor. Nodal size is normally reported as two dimensions in the plane in which the image is obtained (for CT scan this is almost always the axial plane; for MRI the plane of acquisition may be axial, sagittal or coronal). The smaller of these measures is the short axis.

For example, an abdominal node which is reported as being 20 mm. 30 mm has a short axis of 20 mm and qualifies as a malignant, measurable node. In this example, 20 mm should be recorded as the node measurement. All other pathological nodes (those with short axis P10 mm but <15 mm) should be considered non-target lesions. Nodes that have a short axis <10 mm are considered non-pathological and should not be recorded or followed.

A sum of the diameters (longest for non-nodal lesions, short axis for nodal lesions) for all target lesions will be calculated and reported as the baseline sum diameters. If lymph nodes are to be included in the sum, then as noted above, only the short axis is added into the sum. The baseline sum diameters will be used as reference to further characterize any objective tumor regression in the measurable dimension of the disease.

All other lesions (or sites of disease) including pathological lymph nodes should be identified as non-target lesions and should also be recorded at baseline. Measurements are not required and these lesions should be followed as ‘present’, ‘absent’, or in rare cases ‘unequivocal progression.’ In addition, it is possible to record multiple nontarget lesions involving the same organ as a single item on the case record form (e.g., ‘multiple enlarged pelvic lymph nodes’ or ‘multiple liver metastases’).

In some embodiments, tumor response can be measured by, for example, the immune-related RECIST (irRECIST) guidelines, which include immune related Response Criteria (irRC). In irRC, measurable lesions are measured that have at least one dimension with a minimum size of 10 mm (in the longest diameter by CT or MRI scan) for nonnodal lesions and greater than or equal to 15 mm for nodal lesions, or at least 20 mm by chest X-ray.

In some embodiments, Immune Related Response Criteria include CR (complete disappearance of all lesions (measurable or not, and no new lesions)); PR (decrease in tumor burden by 50% or more relative to baseline); SD (not meeting criteria for CR or PR in the absence of PD); or PD (an increase in tumor burden of at 25% or more relative to nadir). Detailed description of irRECiST can be found at Bohnsack et al., (2014) ESMO. ABSTRACT 4958 and Nishino et al., (2013) Clin. Cancer Res. 19(14): 3936-43.

In some embodiments, tumor response can be assessed by either irRECIST or RECIST version 1.1. In some embodiments, tumor response can be assessed by both irRECIST and RECIST version 1.1.

### II. Anti-CTLA4 Binding Proteins

Provided herein are proteins that bind cytotoxic T-lymphocyte-associated protein 4 (CTLA4). In some embodiments, the provided CTLA4 binding protein is an antibody or an antigen-binding fragment thereof, or is a protein that comprises a CTLA4 binding domain. In some embodiments, the CTLA4 binding protein is an anti-CTLA4 antibody or antigen-binding fragment thereof. In some embodiments, the CTLA4 binding protein is a bispecific antibody, or an antigen-binding fragment thereof, that binds to CTLA4. For example, the bispecific antibody that contains a CTLA binding domain. In one aspect, there is provided a CTLA4 binding protein comprising a CTLA4 binding domain, such as a fusion protein that comprises a CTLA4 binding domain described herein. In some embodiments, the CTLA4 binding protein is a chimeric receptor that binds to CTLA4, for example, by virtue of an antigen-binding domain that can bind CTLA4.

The CTLA4 binding proteins provided herein can bind to CTLA4 from various species, for example, some bind to a human CTLA4 and/or murine CTLA4, or cynomolgus CTLA4. In some embodiments, an anti-CTLA4 binding protein described herein has one or more of the following characteristics: (1) binds a CTLA4 (e.g. a human CTLA4); and (2) binds a CTLA4 in vivo at a tumor site.

In one aspect, provided herein are CTLA4 binding proteins useful, inter alia, for the treatment of a neoplastic disease in which CTLA4 plays a role. An CTLA4 binding protein as provided herein includes a binding domain capable of interacting with (e.g., binding to) a CTLA4 protein expressed on the surface of a cell (e.g., a cancer cell or T cell).

Also provided herein, in some embodiments, is a CTLA4 binding protein (e.g., an anti-CTLA4 antibody or antigen-binding fragment thereof) comprising a CTLA4 binding protein (e.g., an anti-CTLA4 antibody or antigen-binding fragment thereof comprising a first chain and a second chain). In some embodiments, the CTLA4 binding protein is a dimer. In some embodiments, the CTLA4 binding protein is a homodimer. In some embodiments, the CTLA4 binding protein is a heterodimer. In some embodiments, the CTLA4 binding protein is a heterodimer comprising a first chain and a second chain, such as a heterodimer comprising a heavy chain and a light chain. In some embodiments, the CTLA4 binding protein is an anti-CTLA4 antibody or antigen-binding fragment thereof comprising a first chain and a second chain. In some embodiments, the first chain is or comprises a heavy chain, and the second chain is or comprises a light chain; or the first chain is or comprises a light chain, and the second chain is or comprises a heavy chain. In some embodiments, the first chain is or comprises a heavy chain variable region, and the second chain is or comprises a light chain variable region; or the first chain is or comprises a light chain variable region, and the second chain is or comprises a heavy chain variable region. In some embodiments, the CTLA4 binding protein is a single chain protein, such as a single chain protein that comprises both a heavy chain and a light chain.

Also provided is a nucleic acid encoding the antibodies, the bispecific antibodies, or any antigen-binding fragments thereof, or the chimeric receptor of any one the aforementioned embodiments. Also provided is a vector comprising the nucleic acid of the aforementioned embodiments. In some embodiments, the vector is an expression vector. Also provided is a host cell comprising the aforementioned nucleic acid embodiments.

Also provided is a method of producing an antibody, a bispecific antibody, or any antigen-binding fragment thereof, or a chimeric receptor comprising culturing the aforementioned host cells under a condition that produces the antibody, bispecific antibody or chimeric receptor. In some embodiments, the host cell has an alpha1,6-fucosyltransferase (Fut8) knockout. In some embodiments, wherein the host cell overexpresses β1,4-N-acetylglycosminyltransferase III (GnT-III). In some embodiments, the host cell additionally overexpresses Golgi μ-mannosidase II (ManII). Some of any such embodiments, further include recovering the antibody, bispecific antibody or any antigen-binding fragments thereof or chimeric receptor produced by the host cell. In some embodiments, bispecific antibody or chimeric receptor produced by the aforementioned methods.

Also provided is a composition containing an antibody, a bispecific antibody or any antigen-binding fragments thereof, or a chimeric receptor of any one of aforementioned embodiments. Some embodiments encompass a composition comprising the antibody, bispecific antibody or any antigen-binding fragments thereof or chimeric receptor of the aforementioned embodiments. In some embodiments, the composition is a pharmaceutical composition.

Also provided is a kit containing the antibody, the bispecific antibody or any antigen-binding fragments thereof, the chimeric receptor, or the composition of any one of aforementioned embodiments.

Also provided is a method of treating or preventing a neoplastic disease in a subject, the method comprising administering to the subject an effective amount of the antibody, the bispecific antibody or any antigen-binding fragments thereof, the chimeric receptor, or the composition of any one of aforementioned embodiments. In one embodiment, the neoplastic disease is a cancer. In some embodiments, the cancer is leukemia, lymphoma, head and neck cancer, colorectal cancer, prostate cancer, pancreatic cancer, melanoma, breast cancer, neuroblastoma, lung cancer, ovarian cancer, osteosarcoma, bladder cancer, cervical cancer, liver cancer, kidney cancer, skin cancer or testicular cancer.

### CTLA4 Binding Protein

The term “CTLA4 binding protein” as provided herein refers to a polypeptide comprising a CTLA4 binding domain that is capable of binding to, or otherwise exhibiting an affinity for, a CTLA4 protein. In some embodiments, the CTLA4 binding protein is an anti-CTLA4 antibody or antigen-binding fragment thereof, a bispecific antibody, an antigen binding fragment, a single chain antibody, etc. In some embodiments, the CTLA4 binding protein is an antibody or antigen-binding fragment thereof that binds to CTLA4. In some embodiments, an antibody or antigen-binding fragment thereof that binds to CTLA4 is an anti-CTLA4 antibody or antigen-binding fragment thereof. Accordingly, in some embodiments, the CTLA4 binding protein is an anti-CTLA4 antibody or antigen-binding fragment thereof. In some embodiments, the CTLA4 binding protein is a component of a chimeric antigen receptor that binds CTLA4.

The term “CTLA4 binding domain” refers to a recombinantly expressed polypeptide domain capable of binding to, or otherwise exhibiting an affinity for, a CTLA4 protein found in or on a cell. Methods for determining the extent of binding of a CTLA4 binding domain to CTLA4 are well known in the art.

In some embodiments, the CTLA4 binding domain provided herein is an antibody capable of binding to CTLA4. In some embodiments, the CTLA4 is a human CTLA4. In some embodiments, the CTLA4 is a murine CTLA4. In some embodiments, the antibody is a murine antibody.

In some embodiments, the antibody is a humanized antibody, a chimeric antibody, or a human antibody. In some embodiments, an anti-CTLA4 antibody or antigen-binding fragment thereof described herein is a monoclonal antibody. In some embodiments, an anti-CTLA4 antibody or antigen-binding fragment thereof described herein is an antibody fragment (including antigen-binding fragment). e.g., a dAb, Fab, Fab′-SH, Fv, scFv, or (Fab′)2 fragment. In some embodiments, the antibody or antigen-binding fragment thereof is a dimer. In some embodiments, the antibody or antigen-binding fragment thereof is a homodimer. In some embodiments, the antibody or antigen-binding fragment thereof is a heterodimer. In some embodiments, the antibody or antigen-binding fragment thereof is a heterodimer comprising a first chain and a second chain, such as a heterodimer comprising a heavy chain and a light chain. In some embodiments, the antibody or antigen-binding fragment comprises a first chain and a second chain. In some embodiments, the first chain is or comprises a heavy chain, and the second chain is or comprises a light chain; or the first chain is or comprises a light chain, and the second chain is or comprises a heavy chain. In some embodiments, the first chain is or comprises a heavy chain variable region, and the second chain is or comprises a light chain variable region; or the first chain is or comprises a light chain variable region, and the second chain is or comprises a heavy chain variable region. In some embodiments, the antibody or antigen-binding fragment thereof comprises a first chain and a second chain (e.g., a light chain and a heavy chain). In some embodiments, the antibody or antigen-binding fragment thereof comprises two first chains and two second chains (e.g., two light chains and two heavy chains). In some embodiments, the antibody or antigen-binding fragment thereof comprises a light chain variable region and a heavy chain variable region, wherein the light chain variable region comprises (i) a CDR-L1 comprising the amino acid sequence of SEQ ID NO:1 or 13, (ii) a CDR-L2 comprising the amino acid sequence of SEQ ID NO:2 or 14, and (iii) a CDR-L3 comprising the amino acid sequence of SEQ ID NO:3 or 15; and/or wherein the heavy chain variable region comprises (i) a CDR-H1 comprising the amino acid sequence of SEQ ID NO:4 or 16, (ii) a CDR-H2 comprising the amino acid sequence of SEQ ID NO:5 or 17, and (iii) a CDR-H3 comprising the amino acid sequence of SEQ ID NO:6 or 18.

In some embodiments, the antibody or antigen-binding fragment comprises a light chain variable region and a heavy chain variable region, wherein the light chain variable region comprises (i) a CDR-L1 comprising the amino acid sequence of SEQ ID NO:1, (ii) a CDR-L2 comprising the amino acid sequence of SEQ ID NO:2, and (iii) a CDR-L3 comprising the amino acid sequence of SEQ ID NO:3; and/or wherein the heavy chain variable region comprises (i) a CDR-H1 comprising the amino acid sequence of SEQ ID NO:4, (ii) a CDR-H2 comprising the amino acid sequence of SEQ ID NO:5, and (iii) a CDR-H3 comprising the amino acid sequence of SEQ ID NO:6. In some embodiments, the antibody or antigen-binding fragment comprises a light chain variable region and a heavy chain variable region, wherein the light chain variable region comprises (i) a CDR-L1 comprising the amino acid sequence of SEQ ID NO:1, (ii) a CDR-L2 comprising the amino acid sequence of SEQ ID NO:2, and (iii) a CDR-L3 comprising the amino acid sequence of SEQ ID NO:3; and the heavy chain variable region comprises (i) a CDR-H1 comprising the amino acid sequence of SEQ ID NO:4, (ii) a CDR-H2 comprising the amino acid sequence of SEQ ID NO:5, and (iii) a CDR-H3 comprising the amino acid sequence of SEQ ID NO:6.

In some embodiments, the antibody or antigen-binding fragment comprises a light chain variable region and a heavy chain variable region, wherein the light chain variable region comprises (i) a CDR-L1 comprising the amino acid sequence of SEQ ID NO:7, (ii) a CDR-L2 comprising the amino acid sequence of SEQ ID NO:8, and (iii) a CDR-L3 comprising the amino acid sequence of SEQ ID NO:9; and/or wherein the heavy chain variable region comprises (i) a CDR-H1 comprising the amino acid sequence of SEQ ID NO:10, (ii) a CDR-H2 comprising the amino acid sequence of SEQ ID NO:11, and (iii) a CDR-H3 comprising the amino acid sequence of SEQ ID NO:12. In some embodiments, the antibody or antigen-binding fragment comprises a light chain variable region and a heavy chain variable region, wherein the light chain variable region comprises (i) a CDR-L1 comprising the amino acid sequence of SEQ ID NO:7, (ii) a CDR-L2 comprising the amino acid sequence of SEQ ID NO:8, and (iii) a CDR-L3 comprising the amino acid sequence of SEQ ID NO:9; and the heavy chain variable region comprises (i) a CDR-H1 comprising the amino acid sequence of SEQ ID NO:10, (ii) a CDR-H2 comprising the amino acid sequence of SEQ ID NO:11, and (iii) a CDR-H3 comprising the amino acid sequence of SEQ ID NO:12.

In some embodiments, the antibody or antigen-binding fragment comprises a light chain variable region and a heavy chain variable region, wherein the light chain variable region comprises (i) a CDR-L1 comprising the amino acid sequence of SEQ ID NO:13, (ii) a CDR-L2 comprising the amino acid sequence of SEQ ID NO:14, and (iii) a CDR-L3 comprising the amino acid sequence of SEQ ID NO:15; and/or wherein the heavy chain variable region comprises (i) a CDR-H1 comprising the amino acid sequence of SEQ ID NO:16, (ii) a CDR-H2 comprising the amino acid sequence of SEQ ID NO:17, and (iii) a CDR-H3 comprising the amino acid sequence of SEQ ID NO:18. In some embodiments, the antibody or antigen-binding fragment comprises a light chain variable region and a heavy chain variable region, wherein the light chain variable region comprises (i) a CDR-L1 comprising the amino acid sequence of SEQ ID NO:13, (ii) a CDR-L2 comprising the amino acid sequence of SEQ ID NO:14, and (iii) a CDR-L3 comprising the amino acid sequence of SEQ ID NO:15; and the heavy chain variable region comprises (i) a CDR-H1 comprising the amino acid sequence of SEQ ID NO:16, (ii) a CDR-H2 comprising the amino acid sequence of SEQ ID NO:17, and (iii) a CDR-H3 comprising the amino acid sequence of SEQ ID NO:18.

In some embodiments, the antibody or antigen-binding fragment comprises a light chain variable region and a heavy chain variable region, wherein the light chain variable region comprises (i) a CDR-L1 comprising the amino acid sequence of SEQ ID NO:19, (ii) a CDR-L2 comprising the amino acid sequence of SEQ ID NO:20, and (iii) a CDR-L3 comprising the amino acid sequence of SEQ ID NO:21; and/or wherein the heavy chain variable region comprises (i) a CDR-H1 comprising the amino acid sequence of SEQ ID NO:22, (ii) a CDR-H2 comprising the amino acid sequence of SEQ ID NO:23, and (iii) a CDR-H3 comprising the amino acid sequence of SEQ ID NO:24. In some embodiments, the antibody or antigen-binding fragment comprises a light chain variable region and a heavy chain variable region, wherein the light chain variable region comprises (i) a CDR-L1 comprising the amino acid sequence of SEQ ID NO:19, (ii) a CDR-L2 comprising the amino acid sequence of SEQ ID NO:20, and (iii) a CDR-L3 comprising the amino acid sequence of SEQ ID NO:21; and the heavy chain variable region comprises (i) a CDR-H1 comprising the amino acid sequence of SEQ ID NO:22, (ii) a CDR-H2 comprising the amino acid sequence of SEQ ID NO:23, and (iii) a CDR-H3 comprising the amino acid sequence of SEQ ID NO:24.

In some embodiments, the VL domain comprises a CDR-L1, a CDR-L2 and a CDR-L3 contained within the amino acid sequence of SEQ ID NO: 25 and the VH domain comprises a CDR-H1, a CDR-H2 and a CDR-H3 contained within the amino acid sequence of SEQ ID NO: 26. In some embodiments, the VL domain comprises a CDR-L1, a CDR-L2 and a CDR-L3 contained within the amino acid sequence of SEQ ID NO: 30 and the VH domain comprises a CDR-H1, a CDR-H2 and a CDR-H3 contained within the amino acid sequence of SEQ ID NO: 31.

In some embodiments, the first chain is a light chain; and the second chain is a heavy chain. In some embodiments, the antibody or antigen-binding fragment thereof containing two first chains and two second chains. In some embodiments, the first chain is a light chain variable domain; and the second chain is a heavy chain variable domain. In some of any such embodiments, the antigen-binding fragment is a dAb, Fab, Fab′-SH, Fv, scFv, or (Fab′)2 fragment.

In some of any such embodiments, the antibody is a murine antibody.

In some of any such embodiments, the antibody is a humanized antibody, a chimeric antibody, or a human antibody. In some of any such embodiments, the antibody has an IgG1, IgG2, IgG3 or IgG4 isotype. In some of any such embodiments, the IgG1 contain the amino acid substitutions, S298A, E333A, and K334A; S239D and I332E; S239D, A330L, and I332E; P247I and A339D or A339Q; D280H, K290S with or without S298D or S298V; F243L., R292P, and Y300L; F243L, R292P, Y300L, and P396L; F2431, R292P, Y300L, V305I, and P396L; G236A, S239D, and I332E; K326A and E333A; K326W and E333S: or K290E or K290N, S298G, T299A, and/or K326E; wherein the amino acid residues am numbered according to the EU index as in Kabat.

In some of any such embodiments, the antibody or antigen-binding fragment containing a light chain variable region comprising the amino acid sequence of SEQ ID NO:25 or 30; and/or a heavy chain variable region comprising the amino acid sequence of SEQ ID NO:26 or 31.

In some of any embodiments, the VL domain comprises an amino acid sequence that has at least about 90% sequence identity to the amino acid sequence of SEQ ID NO: 25, and the VH domain comprises an amino acid sequence that has at least about 90% sequence identity to the amino acid sequence of SEQ ID NO: 26. In some of any such embodiments, the antibody or antigen-binding fragment containing a light chain variable region comprising the amino acid sequence of SEQ ID NO:25 and a heavy chain variable region comprising the amino acid sequence of SEQ ID NO:26.

In some of any embodiments, the VL domain comprises an amino acid sequence that has at least about 90% sequence identity to the amino acid sequence of SEQ ID NO: 30, and the VH domain comprises an amino acid sequence that has at least about 90% sequence identity to the amino acid sequence of SEQ ID NO: 31. In some of any such embodiments, the antibody or antigen-binding fragment containing a light chain variable region comprising the amino acid sequence of SEQ ID NO:30 and a heavy chain variable region comprising the amino acid sequence of SEQ ID NO:31.

In some embodiments, the antibody or antigen-binding fragment thereof comprises a light chain comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 27; and/or comprises a heavy chain comprising an amino acid sequence having or having about 70%, 75%, 80%, 85%, 90%, 91%, 92%, 93%, 94%, 95%, %%, 97%, 98%, 99% or 100% homology to the amino acid sequence of SEQ ID NO: 28. In some embodiments, the antibody or antigen-binding fragment thereof comprises a light chain comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 27; and comprises a heavy chain comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 28. In some embodiments, the antibody or antigen-binding fragment thereof comprises a light chain comprising the amino acid sequence of SEQ ID NO: 27; and/or comprises a heavy chain comprising the amino acid sequence of SEQ ID NO: 28. In some embodiments, the antibody or antigen-binding fragment thereof comprises a light chain comprising the amino acid sequence of SEQ ID NO: 27; and comprises a heavy chain comprising the amino acid sequence of SEQ ID NO: 28.

In some embodiments, the antibody or antigen-binding fragment thereof comprises a light chain comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 27; and/or comprises a heavy chain comprising an amino acid sequence having or having about 70%, 75%, 80%, 85%, 90%, 91%, 92%, 93%, 94%, 95%, %%, 97%, 98%, 99% or 100% homology to the amino acid sequence of SEQ ID NO: 29. In some embodiments, the antibody or antigen-binding fragment thereof comprises a light chain comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 27; and comprises a heavy chain comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 29. In some embodiments, the antibody or antigen-binding fragment thereof comprises a light chain comprising the amino acid sequence of SEQ ID NO: 27; and/or comprises a heavy chain comprising the amino acid sequence of SEQ ID NO: 29. In some embodiments, the antibody or antigen-binding fragment thereof comprises a light chain comprising the amino acid sequence of SEQ ID NO: 27; and comprises a heavy chain comprising the amino acid sequence of SEQ ID NO: 29.

In some embodiments, the antibody or antigen-binding fragment thereof comprises a light chain comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 32; and/or comprises a heavy chain comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 33. In some embodiments, the antibody or antigen-binding fragment thereof comprises a light chain comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 32; and comprises a heavy chain comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 33. In some embodiments, the antibody or antigen-binding fragment thereof comprises a light chain comprising the amino acid sequence of SEQ ID NO: 32; and/or comprises a heavy chain comprising the amino acid sequence of SEQ ID NO: 33. In some embodiments, the antibody or antigen-binding fragment thereof comprises a light chain comprising the amino acid sequence of SEQ ID NO: 32; and comprises a heavy chain comprising the amino acid sequence of SEQ ID NO: 33.

In some embodiments, the antibody or antigen-binding fragment thereof comprises a light chain comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 32; and/or comprises a heavy chain comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 34. In some embodiments, the antibody or antigen-binding fragment thereof comprises a light chain comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 32; and comprises a heavy chain comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 34. In some embodiments, the antibody or antigen-binding fragment thereof comprises a light chain comprising the amino acid sequence of SEQ ID NO: 32; and/or comprises a heavy chain comprising the amino acid sequence of SEQ ID NO: 34. In some embodiments, the antibody or antigen-binding fragment thereof comprises a light chain comprising the amino acid sequence of SEQ ID NO: 32; and comprises a heavy chain comprising the amino acid sequence of SEQ ID NO: 34.

some embodiments, the antibody or antigen-binding fragment thereof comprises a light chain comprising the amino acid sequence selected from SEQ ID NOs:27 and 32; and/or a heavy chain comprising the amino acid sequence selected from SEQ ID NOs:28, 29, 33 and 34.

In some of any such embodiments, the antibody or antigen-binding fragment thereof contains a light chain comprising the amino acid sequence of SEQ ID NO:27; and/or a heavy chain comprising the amino acid sequence of SEQ ID NO:28. In some of any such embodiments, the antibody or antigen-binding fragment thereof contains a light chain comprising the amino acid sequence of SEQ ID NO:27; and/or a heavy chain comprising the amino acid sequence of SEQ ID NO:29. In some of any such embodiments, the antibody or antigen-binding fragment thereof contains a light chain comprising the amino acid sequence of SEQ ID NO:27; and a heavy chain comprising the amino acid sequence of SEQ ID NO:28. In some of any such embodiments, the antibody or antigen-binding fragment thereof contains a light chain comprising the amino acid sequence of SEQ ID NO:27; and a heavy chain comprising the amino acid sequence of SEQ ID NO:29.

In some of any such embodiments, the antibody or antigen-binding fragment thereof contains a light chain comprising the amino acid sequence of SEQ ID NO:32; and/or a heavy chain comprising the amino acid sequence of SEQ ID NO:33. In some of any such embodiments, the antibody or antigen-binding fragment thereof contains a light chain comprising the amino acid sequence of SEQ ID NO:32; and/or a heavy chain comprising the amino acid sequence of SEQ ID NO:34. In some of any such embodiments, the antibody or antigen-binding fragment thereof contains a light chain comprising the amino acid sequence of SEQ ID NO:32; and a heavy chain comprising the amino acid sequence of SEQ ID NO:33. In some of any such embodiments, the antibody or antigen-binding fragment thereof contains a light chain comprising the amino acid sequence of SEQ ID NO:32; and a heavy chain comprising the amino acid sequence of SEQ ID NO:34.

Also provide herein is a bispecific antibody containing a light chain and a heavy chain of a first pair that specifically binds to CTLA4 and light chain and a heavy chain of a second pair that specifically binds to an antigen, for example, an antigen that is a tumor antigen.

In some of any such embodiments, the bispecific antibody is a murine antibody.

In some of any such embodiments, the bispecific antibody is a humanized antibody, a chimeric antibody, or a human antibody. In some of any such embodiments, the bispecific antibody has an IgG1, IgG2, IgG3 or IgG4 isotype. In some of any such embodiments, the IgG1 contain the amino acid substitutions, such as S298A, E333A, and K334A; S239D and I332E; S239D, A330L, and I332E; P247I and A339D or A339Q; D280H, K290S with or without S298D or S298V; F243L, R292P, and Y300L; F243L, R292P, Y300L, and P396L; F2431, R292P, Y300L, V305L and P396L; G236A, S239D, and I332E; K326A and E333A; K326W and E333S; or K290E or K290N, S298G, T299A, and/or K326E, wherein the amino acid residues are numbered according to the EU index as in Kabat.

In some of any such embodiments, the first pair contains a light chain variable region and a heavy chain variable region, wherein the light chain variable region containing (i) CDR-L1 comprising the amino acid sequence of SEQ ID NO:1 or 13, (ii) CDR-L2 comprising the amino acid sequence of SEQ ID NO:2 or 14, and (iii) CDR-L3 comprising the amino acid sequence of SEQ ID NO:3 or 15; and/or wherein the heavy chain variable region containing (i) CDR-H1 comprising the amino acid sequence of SEQ ID NO:4 or 16, (ii) CDR-H2 comprising the amino acid sequence of SEQ ID NO:5 or 17, and (iii) CDR-H3 comprising the amino acid sequence of SEQ ID NO:6 or 18.

In some of any such embodiments, the first pair contains a light chain variable region comprising the amino acid sequence of SEQ ID NO:25 or 30; and/or a heavy chain variable region comprising the amino acid sequence of SEQ ID NO:26 or 31.

In some of any such embodiments, the first pair contains a VL domain comprising an amino acid sequence that has at least about 90% sequence identity to the amino acid sequence of SEQ ID NO: 25, and a VH domain comprising an amino acid sequence that has at least about 90% sequence identity to the amino acid sequence of SEQ ID NO: 26. In some of any such embodiments, the first pair contains a light chain variable region comprising the amino acid sequence of SEQ ID NO:25 and a heavy chain variable region comprising the amino acid sequence of SEQ ID NO:26.

In some of any such embodiments, the first pair contains a VL domain comprising an amino acid sequence that has at least about 90% sequence identity to the amino acid sequence of SEQ ID NO: 30, and a VH domain comprising an amino acid sequence that has at least about 90% sequence identity to the amino acid sequence of SEQ ID NO: 31. In some of any such embodiments, the first pair contains a light chain variable region comprising the amino acid sequence of SEQ ID NO:30 and a heavy chain variable region comprising the amino acid sequence of SEQ ID NO:31.

In some of any such embodiments, the first pair contains a light chain comprising the amino acid sequence selected from SEQ ID NOs:27 and 32; and/or a heavy chain comprising the amino acid sequence selected from SEQ ID NOs:28, 29, 33 and 34.

In some of any such embodiments, the first pair contains a light chain comprising the amino acid sequence of SEQ ID NO:27; and/or a heavy chain comprising the amino acid sequence of SEQ ID NO:28. In some of any such embodiments, the first pair contains a light chain comprising the amino acid sequence of SEQ ID NO:27; and/or a heavy chain comprising the amino acid sequence of SEQ ID NO:29. In some of any such embodiments, the first pair contains a light chain comprising the amino acid sequence of SEQ ID NO:27; and a heavy chain comprising the amino acid sequence of SEQ ID NO:28. In some of any such embodiments, the first pair contains a light chain comprising the amino acid sequence of SEQ ID NO:27; and a heavy chain comprising the amino acid sequence of SEQ ID NO:29.

In some of any such embodiments, the first pair contains a light chain comprising the amino acid sequence of SEQ ID NO:32; and/or a heavy chain comprising the amino acid sequence of SEQ ID NO:33. In some of any such embodiments, the first pair contains a light chain comprising the amino acid sequence of SEQ ID NO:32; and/or a heavy chain comprising the amino acid sequence of SEQ ID NO:34. In some of any such embodiments, the first pair contains a light chain comprising the amino acid sequence of SEQ ID NO:32; and a heavy chain comprising the amino acid sequence of SEQ ID NO:33. In some of any such embodiments, the first pair contains a light chain comprising the amino acid sequence of SEQ ID NO:32; and a heavy chain comprising the amino acid sequence of SEQ ID NO:34.

In some of any such embodiments, the bispecific antibody contains a first chain and a second chain, wherein the first chain containing (i) CDR-L1 comprising the amino acid sequence of SEQ ID NO:1 or 13, (ii) CDR-L2 comprising the amino acid sequence of SEQ ID NO:2 or 14, and (iii) CDR-L3 comprising the amino acid sequence of SEQ ID NO:3 or 15; and/or wherein the second chain containing (i) CDR-H1 comprising the amino acid sequence of SEQ ID NO:4 or 16, (ii) CDR-H2 comprising the amino acid sequence of SEQ ID NO:5 or 17, and (iii) CDR-H3 comprising the amino acid sequence of SEQ ID NO:6 or 18.

In some of any such embodiments, the first chain contains a light chain variable region comprising the amino acid sequence of SEQ ID NO:25 or 30; and/or a heavy chain variable region comprising the amino acid sequence of SEQ ID NO:26 or 31.

In some of any such embodiments, the first chain contains a VL domain comprising an amino acid sequence that has at least about 90% sequence identity to the amino acid sequence of SEQ ID NO: 25, and a VH domain comprising an amino acid sequence that has at least about 90% sequence identity to the amino acid sequence of SEQ ID NO: 26. In some of any such embodiments, the first chain contains a light chain variable region comprising the amino acid sequence of SEQ ID NO:25 and a heavy chain variable region comprising the amino acid sequence of SEQ ID NO:26.

In some of any such embodiments, the first chain contains a VL domain comprising an amino acid sequence that has at least about 90% sequence identity to the amino acid sequence of SEQ ID NO: 30, and a VH domain comprising an amino acid sequence that has at least about 90% sequence identity to the amino acid sequence of SEQ ID NO: 31. In some of any such embodiments, the first chain contains a light chain variable region comprising the amino acid sequence of SEQ ID NO:30 and a heavy chain variable region comprising the amino acid sequence of SEQ ID NO:31.

In some embodiments, the antibody or antigen-binding fragment comprises a light chain variable region comprising the amino acid sequence of SEQ ID NO:25 or 30; and/or a heavy chain variable region comprising the amino acid sequence of SEQ ID NO:26 or 31. In some embodiments, the antibody or antigen-binding fragment comprises a light chain variable region comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 25; and/or comprises a heavy chain variable region comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 26. In some embodiments, the antibody or antigen-binding fragment comprises a light chain variable region comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 25; and comprises a heavy chain variable region comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 26. In some embodiments, the antibody or antigen-binding fragment comprises a light chain variable region comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 30; and/or comprises a heavy chain variable region comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 31. In some embodiments, the antibody or antigen-binding fragment comprises a light chain variable region comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 30; and comprises a heavy chain variable region comprising an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 31. In some embodiments, the antibody or antigen-binding fragment comprises a light chain variable region comprising the amino acid sequence of SEQ ID NO: 30; and/or comprises a heavy chain variable region comprising the amino acid sequence of SEQ ID NO: 31. In some embodiments, the antibody or antigen-binding fragment comprises a light chain variable region comprising the amino acid sequence of SEQ ID NO: 30; and comprises a heavy chain variable region comprising the amino acid sequence of SEQ ID NO: 31.

In some embodiments, the antibody or antigen-binding fragment thereof has an IgG1, IgG2, IgG3 or IgG4 isotype. In some embodiments, the antibody or antigen-binding fragment thereof has an IgG1 isotype comprising amino acid substitutions that enhance effector function as described herein.

In some embodiments, the CTLA4 binding domain comprises a light chain and a heavy chain of an antigen-binding arm of a bispecific antibody. In some embodiments of the bispecific antibody, the light chain comprises (i) CDR-L1 comprising the amino acid sequence of SEQ ID NO:1 or 13, (ii) CDR-L2 comprising the amino acid sequence of SEQ ID NO:2 or 14, and (iii) CDR-L3 comprising the amino acid sequence of SEQ ID NO:3 or 15; and/or wherein the heavy chain comprises (i) CDR-H1 comprising the amino acid sequence of SEQ ID NO:4 or 16, (ii) CDR-H2 comprising the amino acid sequence of SEQ ID NO:5 or 17, and (iii) CDR-H3 comprising the amino acid sequence of SEQ ID NO:6 or 18. In some embodiments of the bispecific antibody, the light chain comprises (i) a CDR-L1 comprising the amino acid sequence of SEQ ID NO: 1, (ii) a CDR-L2 comprising the amino acid sequence of SEQ ID NO:2, and (iii) a CDR-L3 comprising the amino acid sequence of SEQ ID NO:3; and the heavy chain comprises (i) a CDR-H1 comprising the amino acid sequence of SEQ ID NO:4, (ii) a CDR-H2 comprising the amino acid sequence of SEQ ID NO:5, and (iii) a CDR-H3 comprising the amino acid sequence of SEQ ID NO:6. In some embodiments of the bispecific antibody, the light chain comprises (i) a CDR-L1 comprising the amino acid sequence of SEQ ID NO:7, (ii) a CDR-L2 comprising the amino acid sequence of SEQ ID NO:8, and (iii) a CDR-L3 comprising the amino acid sequence of SEQ ID NO:9; and the heavy chain comprises (i) a CDR-H1 comprising the amino acid sequence of SEQ ID NO:10, (ii) a CDR-H2 comprising the amino acid sequence of SEQ ID NO:11, and (iii) a CDR-H3 comprising the amino acid sequence of SEQ ID NO:12. In some embodiments of the bispecific antibody, the light chain comprises (i) a CDR-L1 comprising the amino acid sequence of SEQ ID NO:13, (ii) a CDR-L2 comprising the amino acid sequence of SEQ ID NO:14, and (iii) a CDR-L3 comprising the amino acid sequence of SEQ ID NO 15; and the heavy chain comprises (i) a CDR-H1 comprising the amino acid sequence of SEQ ID NO:16, (ii) a CDR-H2 comprising the amino acid sequence of SEQ ID NO:17, and (iii) a CDR-H3 comprising the amino acid sequence of SEQ ID NO:18. In some embodiments of the bispecific antibody, the light chain comprises (i) a CDR-L1 comprising the amino acid sequence of SEQ ID NO:19, (ii) a CDR-L2 comprising the amino acid sequence of SEQ ID NO:20, and (iii) a CDR-L3 comprising the amino acid sequence of SEQ ID NO:21; and the heavy chain comprises (i) a CDR-H1 comprising the amino acid sequence of SEQ ID NO:22, (ii) a CDR-H2 comprising the amino acid sequence of SEQ ID NO:23, and (iii) a CDR-H3 comprising the amino acid sequence of SEQ ID NO:24.

In some embodiments of the bispecific antibody, the VL domain comprises a CDR-L, a CDR-L2 and a CDR-L3 contained within the amino acid sequence of SEQ ID NO: 25 and the VH domain comprises a CDR-H1, a CDR-H2 and a CDR-H3 contained within the amino acid sequence of SEQ ID NO: 26. In some embodiments of the bispecific antibody, the VL domain comprises a CDR-L1, a CDR-L2 and a CDR-L3 contained within the amino acid sequence of SEQ ID NO: 30 and the VH domain comprises a CDR-H1, a CDR-H2 and a CDR-H3 contained within the amino acid sequence of SEQ ID NO: 31.

In some embodiments of the bispecific antibody, the light chain comprises an amino acid sequence having or having about 70%, 75%, 80%, 85%, 90%, 91%, 92%, 93%, 94%, 95%, %%, 97%, 98%, 99% or 100% homology to the amino acid sequence of SEQ ID NO: 25; and/or the heavy chain comprises an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 26. In some embodiments of the bispecific antibody, the light chain comprises an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 30; and/or the heavy chain comprises an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 31.

In some embodiments of the bispecific antibody, the light chain variable region comprising the amino acid sequence of SEQ ID NO:25 or 30; and/or the heavy chain variable region comprising the amino acid sequence of SEQ ID NO:26 or 31.

In some of any embodiments of the bispecific antibody, the VL domain comprises an amino acid sequence that has at least about 90% sequence identity to the amino acid sequence of SEQ ID NO: 25, and the VH domain comprises an amino acid sequence that has at least about 90% sequence identity to the amino acid sequence of SEQ ID NO: 26. In some of any such embodiments of the bispecific antibody, the antibody or antigen-binding fragment containing a light chain variable region comprising the amino acid sequence of SEQ ID NO:25 and a heavy chain variable region comprising the amino acid sequence of SEQ ID NO:26.

In some of any embodiments of the bispecific antibody, the VL domain comprises an amino acid sequence that has at least about 90% sequence identity to the amino acid sequence of SEQ ID NO: 30, and the VH domain comprises an amino acid sequence that has at least about 90% sequence identity to the amino acid sequence of SEQ ID NO: 31. In some of any such embodiments of the bispecific antibody, the antibody or antigen-binding fragment containing a light chain variable region comprising the amino acid sequence of SEQ ID NO:30 and a heavy chain variable region comprising the amino acid sequence of SEQ ID NO:31.

In some embodiments of the bispecific antibody, the light chain comprises an amino acid sequence having or having about 70%, 75%, 80%, 85%, 90%, 91%, 92%, 93%, 94%, 95%, 96%, 97%, 98%, 99% or 100% homology to an amino acid sequence selected from the group consisting of SEQ ID NOs: 27 and 32; and/or the heavy chain comprises an amino acid sequence having or having about 70%, 75%, 80%, 85%, 90%, 91%, 92%, 93%, 94%, 95%, 96%, 97%, 98%, 99% or 100% homology to an amino acid sequence selected from the group consisting of SEQ ID NOs: 28, 29, 33 and 34. In some embodiments of the bispecific antibody, the light chain comprises the amino acid sequence selected from SEQ ID NOs:27 and 32; and/or the heavy chain comprises the amino acid sequence selected from SEQ ID NOs:28, 29, 33 and 34.

In some embodiments of the bispecific antibody, the light chain comprises an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 27; and/or the heavy chain comprises an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 28. In some embodiments of the bispecific antibody, the light chain comprises the amino acid sequence of SEQ ID NO: 27, and the heavy chain comprises the amino acid sequence of SEQ ID NO: 28.

In some embodiments of the bispecific antibody, the light chain comprises an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 27; and/or the heavy chain comprises an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 29. In some embodiments of the bispecific antibody, the light chain comprises the amino acid sequence of SEQ ID NO: 27, and the heavy chain comprises the amino acid sequence of SEQ ID NO: 29.

In some embodiments of the bispecific antibody, the light chain comprises an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 32; and/or the heavy chain comprises an amino acid sequence having or having about 70%, 75%, 80%, 85%, 90%, 91% 92%, 93%, 94%, 95%, %%, 97%, 98%, 99% or 100% homology to the amino acid sequence of SEQ ID NO: 33. In some embodiments of the bispecific antibody, the light chain comprises the amino acid sequence of SEQ ID NO: 32, and the heavy chain comprises the amino acid sequence of SEQ ID NO: 33.

In some embodiments of the bispecific antibody, the light chain comprises an amino acid sequence having or having about 70%, 75%, 80%, 85%, 90%, 91%, 92%, 93%, 94%, 95%, %%, 97%, 98%, 99% or 100% homology to the amino acid sequence of SEQ ID NO: 32; and/or the heavy chain comprises an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 34. In some embodiments of the bispecific antibody, the light chain comprises the amino acid sequence of SEQ ID NO: 32, and the heavy chain comprises the amino acid sequence of SEQ ID NO: 34.

In some embodiments of the bispecific antibody, the CTLA4 is a human CTLA4. In some embodiments of the bispecific antibody, the CTLA4 is a murine CTLA4. In some embodiments, the bispecific antibody is a murine antibody.

In some embodiments, the bispecific antibody is a humanized antibody, a chimeric antibody, or a human antibody. In some embodiments, the bispecific antibody has an IgG1, IgG2, IgG3 or IgG4 isotype. In some embodiments, the bispecific antibody has an IgG1 isotype comprising amino acid substitutions that enhance effector function as described herein.

In some embodiments, the bispecific antibody or antigen-binding fragment thereof is a dimer. In some embodiments, the bispecific antibody or antigen-binding fragment thereof is a homodimer. In some embodiments, the bispecific antibody or antigen-binding fragment thereof is a heterodimer. In some embodiments, the bispecific antibody or antigen-binding fragment thereof is a heterodimer comprising a first chain and a second chain, such as a heterodimer comprising a heavy chain and a light chain. In some embodiments, the bispecific antibody or antigen-binding fragment comprises a first chain and a second chain. In some embodiments, the first chain is or comprises a heavy chain, and the second chain is or comprises a light chain; or the first chain is or comprises a light chain, and the second chain is or comprises a heavy chain. In some embodiments, the first chain is or comprises a heavy chain variable region, and the second chain is or comprises a light chain variable region; or the first chain is or comprises a light chain variable region, and the second chain is or comprises a heavy chain variable region.

In some embodiments, the CTLA4 binding domain comprises a first chain and a second chain that binds to CTLA4, such as part of a ligand-binding domain for use in a chimeric receptor. In some embodiments of the chimeric receptor, the first chain is a light chain variable domain. In some embodiments, the second chain is a heavy chain variable domain. In some embodiments of the chimeric receptor, the first chain comprises (i) CDR-L1 comprising the amino acid sequence of SEQ ID NO:1 or 13, (ii) CDR-L2 comprising the amino acid sequence of SEQ ID NO:2 or 14, and (iii) CDR-L3 comprising the amino acid sequence of SEQ ID NO:3 or 15; and/or wherein the second chain comprises (i) CDR-H1 comprising the amino acid sequence of SEQ ID NO:4 or 16, (ii) CDR-H2 comprising the amino acid sequence of SEQ ID NO:5 or 17, and (iii) CDR-H3 comprising the amino acid sequence of SEQ ID NO:6 or 18. In some embodiments of the chimeric receptor, the first chain comprises (i) a CDR-L1 comprising the amino acid sequence of SEQ ID NO: 1, (ii) a CDR-L2 comprising the amino acid sequence of SEQ ID NO:2, and (iii) a CDR-L3 comprising the amino acid sequence of SEQ ID NO:3; and the second chain comprises (i) a CDR-H1 comprising the amino acid sequence of SEQ ID NO:4, (ii) a CDR-H2 comprising the amino acid sequence of SEQ ID NO:5, and (iii) a CDR-H3 comprising the amino acid sequence of SEQ ID NO:6. In some embodiments of the chimeric receptor, the first chain comprises (i) a CDR-L1 comprising the amino acid sequence of SEQ ID NO:7, (ii) a CDR-L2 comprising the amino acid sequence of SEQ ID NO:8, and (iii) a CDR-L3 comprising the amino acid sequence of SEQ ID NO:9; and the second chain comprises (i) a CDR-H1 comprising the amino acid sequence of SEQ ID NO:10, (ii) a CDR-H2 comprising the amino acid sequence of SEQ ID NO:11, and (iii) a CDR-H3 comprising the amino acid sequence of SEQ ID NO:12. In some embodiments of the chimeric receptor, the first chain comprises (i) a CDR-L1 comprising the amino acid sequence of SEQ ID NO:13, (ii) a CDR-L2 comprising the amino acid sequence of SEQ ID NO:14, and (iii) a CDR-L3 comprising the amino acid sequence of SEQ ID NO:15; and the second chain comprises (i) a CDR-H1 comprising the amino acid sequence of SEQ ID NO:16, (ii) a CDR-H2 comprising the amino acid sequence of SEQ ID NO:17, and (iii) a CDR-H3 comprising the amino acid sequence of SEQ ID NO:18. In some embodiments of the chimeric receptor, the first chain comprises (i) a CDR-L1 comprising the amino acid sequence of SEQ ID NO:19, (ii) a CDR-L2 comprising the amino acid sequence of SEQ ID NO:20, and (iii) a CDR-L3 comprising the amino acid sequence of SEQ ID NO:21; and the second chain comprises (i) a CDR-H1 comprising the amino acid sequence of SEQ ID NO:22, (ii) a CDR-H2 comprising the amino acid sequence of SEQ ID NO:23, and (iii) a CDR-H3 comprising the amino acid sequence of SEQ ID NO:24.

In some embodiments of the chimeric receptor, the first chain comprises an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 30; and/or the second chain comprises an amino acid sequence having or having about 70%, 75%, 80%, 85%, 90%, 91%, 92%, 93%, 94%, 95%, 96%, 97%, 98%, 99% or 100% homology to the amino acid sequence of SEQ ID NO: 31. In some embodiments of the chimeric receptor, the first chain comprises the amino acid sequence of SEQ ID NO:25 or 30; and/or the second chain comprises the amino acid sequence of SEQ ID NO:26 or 31. In some embodiments of the chimeric receptor, the first chain comprises the amino acid sequence of SEQ ID NO: 30, and the second chain comprises the amino acid sequence of SEQ ID NO: 31.

The CTLA4 binding protein can be conjugated to an additional molecule, by a variety of methods well known in the art. The terms “conjugate” and “conjugate chemistry” refer to reactions with known reactive groups which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, Advanced Organic Chemistry, 3rd Ed., John Wiley & Sons, New York. 1985; Hermanson, Bioconjugate Techniques, Academic Press, San Diego, 1996; and Feeney et al., Modification of Proteins; Advances in Chemistry Series, Vol. 198, American Chemical Society, Washington, D.C., 1982.

Useful reactive functional groups used for conjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc. (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold; (h) amine or sulfhydryl groups, which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (i) metal silicon oxide bonding; and (l) metal bonding to reactive phosphorus groups (e.g. phosphines) to form, for example, phosphate diester bonds.

The reactive functional groups can be chosen such that they do not participate in, or interfere with, the chemical stability of the compositions described herein. Alternatively, a reactive functional group can be protected from participating in the crosslinking reaction by the presence of a protecting group.

In some embodiments, linkers can be engineered to be fused to the additional molecule and/or the CTLA4 binding protein by a variety of methods well known in the art. For example, a nucleic acid can be engineered to encode a linker with an additional molecule and/or a CTLA4 binding protein to produce a fusion protein when recombinantly expressed from a host cell.

### Exemplary CTLA4 Binding Proteins

The following describes certain exemplary embodiments of CTLA4 binding proteins containing certain features as described above. These embodiments are merely exemplary and are not to be construed as being limiting.

Provided herein, in some embodiments, is an antibody or antigen-binding fragment thereof that binds to CTLA4 (e.g., human CTLA4), wherein the antibody or antigen-binding fragment thereof comprises a first chain and a second chain. In some embodiments, the antibody or antigen-binding fragment thereof that binds to CTLA4 is any anti-CTLA4 antibody or antigen-binding fragment thereof described herein. In some embodiments, the antibody or antigen-binding fragment thereof comprises two first chains and two second chains. In some embodiments, the first chain is a light chain and the second chain is a heavy chain. In some embodiments, the first chain is a light chain variable domain and the second chain is a heavy chain variable domain. In some embodiments, the a) the first chain of the antibody is a light chain and the second chain of the antibody is a light chain; b) the first chain of the antibody is a heavy chain and the second chain of the antibody is a heavy chain; or c) the first chain of the antibody is a light chain and the second chain of the antibody is a heavy chain.

Also provided herein, in some embodiments, is an anti-CTLA4 antibody or antigen-binding fragment thereof comprising a light chain variable region comprising a CDR-L1 comprising the amino acid sequence of SEQ ID NO:19, a CDR-L2 comprising the amino acid sequence of SEQ ID NO:20, and a CDR-L3 comprising the amino acid sequence of SEQ ID NO:21; and a heavy chain variable region comprising a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24.

In some embodiments, the light chain variable region comprises the amino acid sequence of SEQ ID NO: 30, and the heavy chain variable region comprises the amino acid sequence of SEQ ID NO: 31. In some embodiments, the anti-CTLA4 antibody or antigen-binding fragment thereof comprises a heavy chain comprising the amino acid sequence of SEQ ID NO: 34 and a light chain comprising the amino acid sequence of SEQ ID NO: 32.

In one aspect, provided herein is an anti-CTLA4 antibody or antigen-binding fragment thereof comprising a light chain variable region comprising the amino acid sequence of SEQ ID NO:25 or 30 and/or a heavy chain variable region comprising the amino acid sequence of SEQ ID NO:26 or 31. In some embodiments, provided herein is an anti-CTLA4 antibody or antigen-binding fragment thereof comprising a light chain variable region comprising the amino acid sequence of SEQ ID NO: 30, and a heavy chain variable region comprising the amino acid sequence of SEQ ID NO: 31.

In a further aspect, provided herein is an anti-CTLA4 antibody or antigen-binding fragment thereof comprising a light chain comprising the amino acid sequence selected from SEQ ID NOs:27 and 32 and/or comprising a heavy chain comprising the amino acid sequence selected from SEQ ID NOs:28, 29, 33 and 34. In some embodiments, provided herein is an anti-CTLA4 antibody or antigen-binding fragment thereof comprising a light chain comprising the amino acid sequence of SEQ ID NO: 27, and a heavy chain comprising the amino acid sequence of SEQ ID NO: 29. In some embodiments, provided herein is an anti-CTLA4 antibody or antigen-binding fragment thereof comprising a light chain comprising the amino acid sequence of SEQ ID NO: 27, and a heavy chain comprising the amino acid sequence of SEQ ID NO: 28. In some embodiments, provided herein is an anti-CTLA4 antibody or antigen-binding fragment thereof comprising a light chain comprising the amino acid sequence of SEQ ID NO: 32, and a heavy chain comprising the amino acid sequence of SEQ ID NO: 33. In some embodiments, provided herein is an anti-CTLA4 antibody or antigen-binding fragment thereof comprising a light chain comprising the amino acid sequence of SEQ ID NO: 32, and a heavy chain comprising the amino acid sequence of SEQ ID NO: 34.

In some embodiments, provided herein is an anti-CTLA4 antibody or antigen-binding fragment thereof comprising a heavy chain variable domain comprising an amino acid sequence having at least 90%, 91%, 92%, 93%, 94%, 95%, 96%, 97%, 98%, or 99% sequence identity to an amino acid sequence selected from SEQ ID NOs:26 and 31. In some embodiments, provided herein is an anti-CTLA4 antibody or antigen-binding fragment thereof comprising a heavy chain variable domain comprising an amino acid sequence having at least 90%, 91%, 92%, 93%, 94%, 95%, 96%, 97%, 98%, or 99% sequence identity to an amino acid sequence of SEQ ID NO:26. In some embodiments, provided herein is an anti-CTLA4 antibody or antigen-binding fragment thereof comprising a heavy chain variable domain comprising an amino acid sequence having at least 90%, 91%, 92%, 93%, 94%, 95%, 96%, 97%, 98%, or 99% sequence identity to an amino acid sequence of SEQ ID NO:31. In some embodiments, an amino acid sequence having at least 90%, 91%, 92%, 93%, 94%, 95%, 96%, 97%, 98%, or 99% sequence identity contains substitutions, insertions, or deletions relative to the reference sequence, but an antibody comprising that amino acid sequence retains the ability to bind to CTLA4 (e.g., human CTLA4). In some embodiments, the substitutions, insertions, or deletions (e.g., 1, 2, 3, 4, or 5 amino acids) occur in regions outside the HVRs (i.e., in the FRs). In some embodiments, an anti-CTLA4 antibody or antigen-binding fragment thereof comprises a heavy chain variable domain comprising an amino acid sequence of SEQ ID NO:26 or 31. In some embodiments, an anti-CTLA4 antibody or antigen-binding fragment thereof comprises a heavy chain variable domain comprising an amino acid sequence of SEQ ID NO:26. In some embodiments, an anti-CTLA4 antibody or antigen-binding fragment thereof comprises a heavy chain variable domain comprising an amino acid sequence of SEQ ID NO:31.

In some embodiments, provided herein is an anti-CTLA4 antibody or antigen-binding fragment thereof comprising a heavy chain variable domain comprising an amino acid sequence having at least 90%, 91%, 92%, 93%, 94%, 95%, 96%, 97%, 98%, or 99% sequence identity to an amino acid sequence of SEQ ID NO:25 or 30. In some embodiments, provided herein is an anti-CTLA4 antibody or antigen-binding fragment thereof comprising a heavy chain variable domain comprising an amino acid sequence having at least 90%, 91%, 92%, 93%, 94%, 95%, 96%, 97%, 98%, or 99% sequence identity to an amino acid sequence of SEQ ID NO:25. In some embodiments, provided herein is an anti-CTLA4 antibody or antigen-binding fragment thereof comprising a heavy chain variable domain comprising an amino acid sequence having at least 90%, 91%, 92%, 93%, 94%, 95%, 96%, 97%, 98%, or 99% sequence identity to an amino acid sequence of SEQ ID NO:30. In some embodiments, an amino acid sequence having at least 90%, 91%, 92%, 93%, 94%, 95%, 96%, 97%, 98%, or 99% sequence identity contains substitutions, insertions, or deletions relative to the reference sequence, but an antibody comprising that amino acid sequence retains the ability to bind to CTLA4 (e.g., human CTLA4). In some embodiments, the substitutions, insertions, or deletions (e.g., 1, 2, 3, 4, or 5 amino acids) occur in regions outside the HVRs (i.e., in the FRs). In some embodiments, an anti-CTLA4 antibody or antigen-binding fragment thereof comprises a heavy chain variable domain comprising an amino acid sequence of SEQ ID NO:25 or 30. In some embodiments, an anti-CTLA4 antibody or antigen-binding fragment thereof comprises a heavy chain variable domain comprising an amino acid sequence of SEQ ID NO:25. In some embodiments, an anti-CTLA4 antibody or antigen-binding fragment thereof comprises a heavy chain variable domain comprising an amino acid sequence of SEQ ID NO:30.

There are five classes of immunoglobulins: IgA, IgD, IgE, IgG and IgM, having heavy chains designated α, δ, ε, γ and μ, respectively. The γ and α classes are further divided into subclasses e.g., humans express the following subclasses: IgG1, IgG2, IgG3, IgG4, IgA1 and IgA2, IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a,f,n,z or combinations thereof. In some of the embodiments herein, the antibody has an IgG1, IgG2, IgG3, or IgG4 isotype. In some embodiments, an anti-CTLA4 antibody or antigen-binding fragment thereof provided herein has an IgG1 isotype (e.g., a human IgG1 isotype). In some embodiments, the antibody provided herein comprises a heavy chain constant domain comprising the amino acid sequence of SEQ ID NO:35. In some embodiments, the antibody provided herein comprises a heavy chain constant domain comprising the amino acid sequence of SEQ ID NO: 38.

In one aspect of the invention, polynucleotides encoding anti-CTLA4 antibodies or antigen-binding fragments thereof are provided. In certain embodiments, vectors comprising polynucleotides encoding anti-CTLA4 antibodies or antigen-binding fragments thereof are provided. In certain embodiments, host cells comprising such vectors are provided. In another aspect of the invention, compositions comprising anti-CTLA4 antibodies described herein or polynucleotides encoding anti-CTLA4 antibodies described herein are provided. In certain embodiments, a composition of the invention is a pharmaceutical formulation for the treatment of a neoplastic disease in which CTLA4 plays a role, such as those enumerated herein.

In some embodiments, the CTLA4 binding protein provided herein is a bispecific antibody capable of binding to CTLA4. Bispecific antibodies are monoclonal antibodies that have binding specificities for at least two different antigens. In some embodiments, one of the binding specificities is for CTLA4 and the other is for any other antigen. In certain embodiments, bispecific antibodies may bind to two different epitopes of CTLA4.

In some aspects, provided herein is a bispecific antibody comprising a) a light chain and a heavy chain of a first pair that specifically binds to CTLA4 and b) a light chain and a heavy chain of a second pair that specifically binds to an antigen. In some aspects, provided herein is a bispecific antibody comprising a) a light chain and a heavy chain of a first pair that specifically binds to CTLA4; and b) a light chain and a heavy chain of a second pair that specifically binds to an antigen. In some embodiments, the antigen is an antigen different from CTLA4. In some embodiments, the light chain of the first pair or the second pair is any light chain described herein. In some embodiments, the heavy chain of the first pair or the second pair is any light chain described herein. In some embodiments, the light chain of the first pair comprises a CDR-L1 comprising the amino acid sequence of SEQ ID NO:19, a CDR-L2 comprising the amino acid sequence of SEQ ID NO:20, and a CDR-L3 comprising the amino acid sequence of SEQ ID NO:21; and the heavy chain of the first pair comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24. In some embodiments, the light chain of the second pair comprises a CDR-L1 comprising the amino acid sequence of SEQ ID NO:19, a CDR-L2 comprising the amino acid sequence of SEQ ID NO:20, and a CDR-L3 comprising the amino acid sequence of SEQ ID NO:21; and the heavy chain of the second pair comprises a CDR-H1 comprising the amino acid sequence of SEQ ID NO:22, a CDR-H2 comprising the amino acid sequence of SEQ ID NO:23, and a CDR-H3 comprising the amino acid sequence of SEQ ID NO:24. In some embodiments, the antigen is to a different epitope of CTLA4.

Bispecific antibodies contemplated herein for use in the bispecific antibodies include murine bispecific antibodies, humanized bispecific antibodies, chimeric bispecific antibodies, and human bispecific antibodies. In some of the embodiments herein, the bispecific antibody has an IgG1, IgG2, IgG3, or IgG4 isotype. In some embodiments, a bispecific antibody provided herein has an IgG1 isotype (e.g., a human IgG1 isotype). In some embodiments, the antibody has an IgG1 isotype comprising amino acid substitutions or is expressed by cells that have no ability to a reduced ability to fucosylate the Fc glycan, that enhance effector function as described herein. In some embodiments, the bispecific antibody provided herein comprises a heavy chain constant domain comprising the amino acid sequence of SEQ ID NO:35. In some embodiments, the bispecific antibody provided herein comprises a heavy chain constant domain comprising the amino acid sequence of SEQ ID NO:38.

In one aspect, provided herein is an anti-CTLA4 bispecific antibody comprising a light chain variable region comprising the amino acid sequence selected from SEQ ID NOs:25 and 30 and/or a heavy chain variable region comprising the amino acid sequence selected from SEQ ID NOs:26 and 31. In a further aspect, provided herein is an anti-CTLA4 bispecific antibody comprising a light chain comprising the amino acid sequence selected from SEQ ID NOs:27 and 32 and/or comprising a heavy chain comprising the amino acid sequence selected from SEQ ID NOs:28, 29, 33 and 34. In some embodiments, provided herein is an anti-CTLA4 bispecific antibody or antigen-binding fragment thereof comprising a light chain comprising the amino acid sequence of SEQ ID NO: 27, and a heavy chain comprising the amino acid sequence of SEQ ID NO: 29. In some embodiments, provided herein is an anti-CTLA4 bispecific antibody or antigen-binding fragment thereof comprising a light chain comprising the amino acid sequence of SEQ ID NO: 27, and a heavy chain comprising the amino acid sequence of SEQ ID NO: 28. In some embodiments, provided herein is an anti-CTLA4 bispecific antibody or antigen-binding fragment thereof comprising a light chain comprising the amino acid sequence of SEQ ID NO: 32, and a heavy chain comprising the amino acid sequence of SEQ ID NO: 33. In some embodiments, provided herein is an anti-CTLA4 bispecific antibody or antigen-binding fragment thereof comprising a light chain comprising the amino acid sequence of SEQ ID NO: 32, and a heavy chain comprising the amino acid sequence of SEQ ID NO: 34.

In some embodiments, provided herein is an anti-CTLA4 bispecific antibody or antigen-binding fragment thereof comprising a heavy chain variable domain comprising an amino acid sequence having at least 90%, 91%, 92%, 93%, 94%, 95%, 96%, 97%, 98%, or 99% sequence identity to an amino acid sequence selected from SEQ ID NOs:26 and 31. In some embodiments, provided herein is an anti-CTLA4 bispecific antibody or antigen-binding fragment thereof comprising a heavy chain variable domain comprising an amino acid sequence having at least 90%, 91%, 92%, 93%, 94%, 95%, 96%, 97%, 98%, or 99% sequence identity to an amino acid sequence of SEQ ID NO:26. In some embodiments, provided herein is an anti-CTLA4 bispecific antibody or antigen-binding fragment thereof comprising a heavy chain variable domain comprising an amino acid sequence having at least 90%, 91%, 92%, 93%, 94%, 95%, 96%, 97%, 98%, or 99% sequence identity to an amino acid sequence of SEQ ID NO:31. In some embodiments, an amino acid sequence having at least 90%, 91%, 92%, 93%, 94%, 95%, 96%, 97%, 98%, or 99% sequence identity contains substitutions, insertions, or deletions relative to the reference sequence, but an antibody comprising that amino acid sequence retains the ability to bind to CTLA4 (e.g., human CTLA4). In some embodiments, the substitutions, insertions, or deletions (e.g., 1, 2, 3, 4, or 5 amino acids) occur in regions outside the HVRs (i.e., in the FRs). In some embodiments, an anti-CTLA4 bispecific antibody or antigen-binding fragment thereof comprises a heavy chain variable domain comprising an amino acid sequence selected from SEQ ID NOs:26 and 31. In some embodiments, an anti-CTLA4 bispecific antibody or antigen-binding fragment thereof comprises a heavy chain variable domain comprising an amino acid sequence of SEQ ID NO:26. In some embodiments, an anti-CTLA4 bispecific antibody or antigen-binding fragment thereof comprises a heavy chain variable domain comprising an amino acid sequence of SEQ ID NO:31.

In some embodiments, provided herein is an anti-CTLA4 bispecific antibody or antigen-binding fragment thereof comprising a heavy chain variable domain comprising an amino acid sequence having at least 90%, 91%, 92%, 93%, 94%, 95%, 96%, 97%, 98%, or 99% sequence identity to an amino acid sequence selected from SEQ ID NOs:25 and 30. In some embodiments, provided herein is an anti-CTLA4 bispecific antibody or antigen-binding fragment thereof comprising a heavy chain variable domain comprising an amino acid sequence having at least 90%, 91%, 92%, 93%, 94%, 95%, 96%, 97%, 98%, or 99% sequence identity to an amino acid sequence of SEQ ID NO:25. In some embodiments, provided herein is an anti-CTLA4 bispecific antibody or antigen-binding fragment thereof comprising a heavy chain variable domain comprising an amino acid sequence having at least 90%, 91%, 92%, 93%, 94%, 95%, 96%, 97%, 98%, or 99% sequence identity to an amino acid sequence of SEQ ID NO:30. In some embodiments, an amino acid sequence having at least 90%, 91%, 92%, 93%, 94%, 95%, 96%, 97%, 98%, or 99% sequence identity contains substitutions, insertions, or deletions relative to the reference sequence, but an antibody comprising that amino acid sequence retains the ability to bind to CTLA4 (e.g., human CTLA4). In some embodiments, the substitutions, insertions, or deletions (e.g., 1, 2, 3, 4, or 5 amino acids) occur in regions outside the HVRs (i.e., in the FRs). In some embodiments, an anti-CTLA4 bispecific antibody or antigen-binding fragment thereof comprises a heavy chain variable domain comprising an amino acid sequence selected from SEQ ID NOs:25 and 30. In some embodiments, an anti-CTLA4 bispecific antibody or antigen-binding fragment thereof comprises a heavy chain variable domain comprising an amino acid sequence of SEQ ID NO:25. In some embodiments, an anti-CTLA4 bispecific antibody or antigen-binding fragment thereof comprises a heavy chain variable domain comprising an amino acid sequence of SEQ ID NO:30.

In some embodiments, the CTLA4 binding protein provided herein is a chimeric receptor (e.g., chimeric antigen receptor (CAR)) capable of binding to CTLA4. CARs are molecules that combine antibody-based specificity for a desired antigen (e.g., CTLA4) with a T cell receptor-activating intracellular domain to generate a chimeric protein that exhibits a specific anti-tumor cellular activity. In one embodiment, provided herein is a chimeric receptor engineered to comprise an extracellular domain having a CTLA4 binding domain described herein fused to an intracellular signaling domain of the T cell antigen receptor complex zeta chain (e.g., CD3 zeta). The chimeric receptor provide herein, when expressed in a T cell is able to redirect antigen recognition based on the antigen binding specificity by virtue of the extracellular domain. In some embodiments, the CTLA4 binding domain is preferably fused with an intracelluar domain from one or more of a costimulatory molecule and a meta chain. In some embodiments, the CTLA4 binding domain is fused with one or more intracellular domains selected from the group of a CD137 (4-1BB) signaling domain, a CD28 signaling domain, a CD3zeta signal domain, and any combination thereof.

In some aspects, provided herein is a chimeric receptor comprising a) a ligand-binding domain comprising a first chain and a second chain that binds to CTLA4; b) a transmembrane domain; and c) an intracellular signaling domain comprising a signaling domain. In some embodiments the first chain is a light chain variable domain and the second chain is a heavy chain variable domain. In some embodiments, the first chain comprises the amino acid sequence selected from SEQ ID NOs:25 and 30; and/or the second chain comprises the amino acid sequence selected from SEQ ID NOs:26 and 31.

In some aspects, provided herein is a chimeric receptor comprising a) a ligand-binding domain comprising a first chain and a second chain that binds to CTLA4; b) a transmembrane domain; and d) an intracellular signaling domain comprising a signaling domain. In some embodiments the first chain is a light chain variable domain and the second chain is a heavy chain variable domain. In some embodiments, the first chain comprises the amino acid sequence selected from SEQ ID NOs:25 and 30; and/or the second chain comprises the amino acid sequence selected from SEQ ID NOs:26 and 31.

In some aspects, provided herein is a chimeric receptor comprising 1) a ligand-binding domain comprising a VL domain and a VH domain that bind to CTLA4, wherein: a) the VL domain comprises a CDR-L1, a CDR-L2 and a CDR-L3 contained within the amino acid sequence of SEQ ID NO: 25 and the VH domain comprises a CDR-H1, a CDR-H2 and a CDR-H3 contained within the amino acid sequence of SEQ ID NO: 26; or b) the VL domain comprises a CDR-L1, a CDR-L2 and a CDR-L3 contained within the amino acid sequence of SEQ ID NO: 30 and the VH domain comprises a CDR-H1, a CDR-H2 and a CDR-H3 contained within the amino acid sequence of SEQ ID NO: 31; 2) a transmembrane domain; and 3) an intracellular signaling domain comprising a signaling domain.

In some aspects, provided herein is a chimeric receptor comprising 1) a ligand-binding domain comprising a VL domain and a VH domain that bind to CTLA4, wherein: a) the VL domain comprises (i) a CDR-L1 comprising the amino acid sequence of SEQ ID NO: 1, (ii) a CDR-L2 comprising the amino acid sequence of SEQ ID NO: 2, and (iii) a CDR-L3 comprising the amino acid sequence of SEQ ID NO: 3; and/or the VH domain comprises (i) a CDR-H1 comprising the amino acid sequence of SEQ ID NO: 4, (ii) a CDR-H2 comprising the amino acid sequence of SEQ ID NO: 5, and (iii) a CDR-H3 comprising the amino acid sequence of SEQ ID NO: 6; or b) the VL domain comprises (i) a CDR-L1 comprising the amino acid sequence of SEQ ID NO: 13, (ii) a CDR-L2 comprising the amino acid sequence of SEQ ID NO: 14, and (iii) a CDR-L3 comprising the amino acid sequence of SEQ ID NO: 15; and/or the VH domain comprises (i) a CDR-H1 comprising the amino acid sequence of SEQ ID NO: 16, (ii) a CDR-H2 comprising the amino acid sequence of SEQ ID NO: 17, and (iii) a CDR-H3 comprising the amino acid sequence of SEQ ID NO: 18; or c) the VL domain comprises (i) a CDR-L1 comprising the amino acid sequence of SEQ ID NO: 7, (ii) a CDR-L2 comprising the amino acid sequence of SEQ ID NO: 8, and (iii) a CDR-L3 comprising the amino acid sequence of SEQ ID NO: 9; and/or the VH domain comprises (i) a CDR-H1 comprising the amino acid sequence of SEQ ID NO: 10, (ii) a CDR-H2 comprising the amino acid sequence of SEQ ID NO: 11, and (iii) a CDR-H3 comprising the amino acid sequence of SEQ ID NO: 12; or d) the VL domain comprises (i) a CDR-L1 comprising the amino acid sequence of SEQ ID NO: 19, (ii) a CDR-L2 comprising the amino acid sequence of SEQ ID NO: 20, and (iii) a CDR-L3 comprising the amino acid sequence of SEQ ID NO: 21; and/or the VH domain comprises (i) a CDR-H1 comprising the amino acid sequence of SEQ ID NO: 22, (ii) a CDR-H2 comprising the amino acid sequence of SEQ ID NO: 23, and (iii) a CDR-H3 comprising the amino acid sequence of SEQ ID NO: 24; 2) a transmembrane domain; and 3) an intracellular signaling domain comprising a signaling domain.

In some aspects, in any of the provided chimeric receptors, a) the VL domain comprises an amino acid sequence that has at least about 90% sequence identity to the amino acid sequence of SEQ ID NO: 25, and the VH domain comprises an amino acid sequence that has at least about 90% sequence identity to the amino acid sequence of SEQ ID NO: 26; or b) the VL domain comprises an amino acid sequence that has at least about 90% sequence identity to the amino acid sequence of SEQ ID NO: 30, and the VH domain comprises an amino acid sequence that has at least about 90% sequence identity to the amino acid sequence of SEQ ID NO: 31.

In some aspects, in any of the provided chimeric receptors, a) the VL domain comprises the amino acid sequence of SEQ ID NO: 25, and the VH domain comprises the amino acid sequence of SEQ ID NO: 26; or b) the VL domain comprises the amino acid sequence of SEQ ID NO: 30, and the VH domain comprises the amino acid sequence of SEQ ID NO: 31.

An exemplary anti-CTLA antibody described herein is Antibody B. Antibody B comprises the following CDR sequences:

| Antibody B | IMGT | Kabat |
|---|---|---|
| HCDR1 | GYTFTNYF (SEQ ID | NYFMN (SEQ ID NO: 22) |
| | NO: 16) | |
| HCDR2 | VDPEQGRAD (SEQ | RVDPEQGRADYAEKFKK |
| | ID NO: 17) | (SEQ ID NO: 23) |
| HCDR3 | RRAMDNYGFAY | RAMDNYGFAY (SEQ ID |
| | (SEQ ID NO: 18) | NO: 24) |
| LCDR1 | SALSYM (SEQ ID | SANSALSYMY (SEQ ID |
| | NO: 13) | NO: 19) |
| LCDR2 | GTS (SEQ ID NO: | GTSNLAS (SEQ ID NO: |
| | 14) | 20) |
| LCDR3 | HHWSNTQ (SEQ ID | HHWSNTQWT (SEQ ID |
| | NO: 15) | NO: 21) |

Antibody B variable light chain:
(SEQ ID NO: 30)
EIVLTQSPDF QSVTPKEKVT ITCSANSALS YMYWYQQKPD QSPKLWVHGT
SNLASGVPSR FSGSGSGTDF TLTINSLEAE DAATYYCHHW SNTQWTFGGG TKVEIK
Antibody B Light Chain
(SEQ ID NO: 32)
EIVLTQSPDFQSVTPKEKVTITCSANSALSYMYWYQQKPDQSPKLWVHGTSNLASGVPSRF
SGSGSGTDFTLTINSLEAEDAATYYCHHWSNTQWTFGGGTKVEIKRTVAAPSVFIFPPSDE
QLKSGTASVVCLLNNFYPREAKVQWKVDNALQSGNSQESVTEQDSKDSTYSLSSTLTLSK
ADYEKHKVYACEVTHQGLSSPVTKSFNRGEC
Antibody B variable heavy chain:
(SEQ ID NO: 31)
QVQLVQSGAE VKKPGSSVKV SCKASGYTFT NYFMNWVRQA PGQGLEWMGR
VDPEQGRADY AEKFKKRVTI TADKSTSTAY MELSSLRSED TAVYYCARRA
MDNYGFAYWG QGTLVTVSS
Antibody B heavy chain:
(SEQ ID NO: 34)
QVQLVQSGAE VKKPGSSVKV SCKASGYTFT NYFMNWVRQA PGQGLEWMGR
VDPEQGRADY AEKFKKRVTI TADKSTSTAY MELSSLRSED TAVYYCARRA
MDNYGFAYWG QGTLVTVSSA STKGPSVFPL APSSKSTSGG TAALGCLVKD
YFPEPVTVSW NSGALTSGVH TFPAVLQSSG LYSLSSVVTV PSSSLGTQTY ICNVNHKPSN
TKVDKKVEPK SCDKTIITCPP CPAPELLGGP DVFLFPPKPK DTLMISRTPE
VTCVVVDVSH EDPEVKFNWY VDGVEVHNAK TKPREEQYNS TYRVVSVLTV
LHQDWLNGKE YKCKVSNKAL PAPEEKTISK AKGQPREPQV YTLPPSRDEL
TKNQVSLTCL VKGFYPSDIA VEWESNGQPE NNYKTTPPVL DSDGSFFLYS
KLTVDKSRWQ QGNVFSCSVM HEALHNHYTQ KSLSLSPGK

### 1. Binding Affinity

The strength, or affinity of immunological binding interactions, such as between an antibody and an antigen for which the antibody is specific, can be expressed in terms of the equilibrium dissociation constant (K_{D}) of the interaction, wherein a smaller Kd represents a greater affinity. Immunological binding properties of proteins can be quantified using methods well known in the art. For example, one method comprises measuring the rates of antigen-binding protein (e.g., antibody)/antigen complex formation and dissociation, wherein those rates depend on the concentrations of the complex partners, the affinity of the interaction, and geometric parameters that equally influence the rate in both directions. Both the “on rate constant” (Kon) and the “off rate constant” (Koff) can be determined by calculation of the concentrations and the actual rates of association and dissociation. The ratio of Koff/Kon enables the cancelation of all parameters not related to affinity, and is equal to the equilibrium dissociation constant K_{D}. See Davies et al., Annual Rev Biochem. 59:439-473, (1990).

In some aspects, an anti-CTLA4 binding protein (e.g., anti-CTLA4 antibody or antigen-binding fragment thereof) described herein binds to CTLA4 with about the same or higher affinity compared to a different anti-CTLA4 binding protein. In certain embodiments, an anti-CTLA4 binding protein provided herein has an equilibrium dissociation constant (K_{D}) of ≤1 μM, ≤150 nM, ≤100 nM, ≤50 nM, ≤10 nM, ≤1 nM, ≤0.1 nM, ≤0.01 nM, or ≤0.001 nM (e.g. 10⁻⁸ M or less, e.g. from 10⁻⁸ M to 101⁻¹³ M, e.g., from 10⁻⁹ M to 10⁻¹³ M). In some embodiments, an anti-CTLA4 binding protein (e.g., an anti-CTLA4 antibody or antigen-binding fragment thereof) provided herein binds to a target protein (e.g., CTLA4 protein) with an equilibrium dissociation constant (K_{D}) of about 50 pM to about 5 nM. Assays for assessing binding affinity are well known in the art, for example such as ELISA, and surface plasma resonance (SPR).

### 2. Biological Activity Assays

In some aspects, an anti-CTLA4 binding protein described herein reduces tumor volume in an in vivo murine tumor model. Assays for assessing reduction of tumor volume are well known in the art.

### III. Anti-CTLA4 Binding Protein Preparation

The anti-CTLA4 binding proteins described herein are prepared using techniques available in the art, exemplary methods of which are described in more detail in the following sections.

### 1. Anti-CTLA4 Binding Protein: Antibody Fragments

The present invention encompasses antibody fragments as anti-CTLA4 binding proteins. Antibody fragments may be generated by traditional means, such as enzymatic digestion, or by recombinant techniques. In certain circumstances there are advantages of using antibody fragments, rather than whole antibodies. For a review of certain antibody fragments, see Hudson et al. (2003) Nat. Med. 9:129-134.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli and other cell types, thus allowing the facile production of large amounts of these fragments. Alternatively, Fab′-SH fragments can be directly recovered from culture media and chemically coupled to form F(ab)2 fragments (Carteret al., Bio/Technology 10: 163-167 (1992)). According to another approach, F(ab′)2 fragments can be isolated directly from recombinant host cell culture. Fab and F(ab′)2 fragment with increased in vivo half-life comprising FCRN/salvage receptor binding epitope residues are described in U.S. Pat. No. 5,869,046. Other techniques for the production of antibody fragments will be apparent to the skilled practitioner. In certain embodiments, an antibody is a single chain Fv fragment (scFv). See WO 93/16185; U.S. Pat. Nos. 5,571,894; and 5,587,458. Fv and scFv are the only species with intact combining sites that are devoid of constant regions; thus, they may be suitable for reduced nonspecific binding during in vivo use. scFv fusion proteins may be constructed to yield fusion of an effector protein at either the amino or the carboxy terminus of an scFv. See Antibody Engineering, ed. Borrebaeck, supra. Also, bi-scFv comprising two scFvs linked via a polypeptide linker can be used as a bispecific antibody. Alternatively, multi-scFv comprising three or more scFvs may be used as a multispecific antibody.

The present invention includes a linear antibody (e.g., as described in U.S. Pat. No. 5,641,870) or a single chain immunoglobulin comprising heavy and light chain sequences of the antibody linked via an appropriate linker. Such linear antibodies or immunoglobulins may be monospecific or bispecific. Such a single chain immunoglobulin can be dimerized to thereby maintain a structure and activities similar to those of the antibody, which is originally a tetramer. Also, the antibody of the present invention may be an antibody that has a single heavy chain variable region and has no light chain sequence. Such an antibody, called a single domain antibody (sdAb) or a nanobody. These antibodies are also encompassed in the meaning of the functional fragment of the antibody according to the present invention.

### 2. Anti-CTLA4 Binding Protein: Humanized Antibodies

The invention encompasses humanized antibodies. Humanized antibodies are generated according to the guidance provided herein. Various methods for humanizing non-human antibodies are known in the art. For example, a humanized antibody can have one or more amino acid residues introduced into it from a source which is non-human. These non-human amino acid residues are often referred to as “import” residues, which are typically taken from an “import” variable domain. Humanization can be essentially performed following the method of Winter (Jones et al. (1986) Nature 321:522-525; Riechmann et al. (1988) Nature 332:323-327; Verhoeyen et al. (1988) Science 239:1534-1536), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

### 3. Anti-CTLA4 Binding Protein: Human Antibodies

Human anti-CTLA4 antibodies of the invention can be constructed by combining Fv clone variable domain sequence(s) selected from human-derived phage display libraries with known human constant domain sequences(s). Alternatively, human monoclonal anti-CTLA4 antibodies of the invention can be made by the hybridoma method. Human myeloma and murine-human heteromyeloma cell lines for the production of human monoclonal antibodies have been described, for example, by Kozbor J. Immunol., 133: 3001 (1984); Brodeur et al., Monoclonal Antibody Production Techniques and Applications, pp. 51-63 (Marcel Dekker. Inc., New York. 1987); and Boerner et al., J. Immunol., 147: 86 (1991). Human antibodies are generated according to the guidance provided herein.

### 4. Anti-CTLA4 Binding Protein: Bispecific Antibodies

Bispecific antibodies are monoclonal antibodies that have binding specificities for at least two different antigens. In certain embodiments, bispecific antibodies are human or humanized antibodies. In certain embodiments, one of the binding specificities is for CTLA4 and the other is for any other antigen. In certain embodiments, bispecific antibodies may bind to two different epitopes of CTLA4. Bispecific antibodies may also be used to localize cytotoxic agents to cells which express CTLA4. Bispecific antibodies can be prepared as full length antibodies or antibody fragments (e.g. F(ab′)2 bispecific antibodies). Bispecific antibodies are generated according to the guidance provided herein.

Methods for making bispecific antibodies are known in the art. See Milstein and Cuello, Nature, 305: 537 (1983), WO 93/08829 published May 13, 1993, Traunecker et al., EMBO J., 10: 3655 (1991); Kontermann and Brinkmann, Drug Discovery Today, 20(7):838-847. For further details of generating bispecific antibodies see, for example, Suresh et al., Methods in Enzymology, 121:210 (1986). Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking method. Suitable cross-linking agents are well known in the art, and are disclosed in U.S. Pat. No. 4,676,980, along with a number of cross-linking techniques.

### 5. Anti-CTLA4 Binding Protein: Single-Domain Antibodies

In some embodiments, a single-domain antibody is generated in accordance with the guidance provided herein. A single-domain antibody is a single polypeptide chain comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1). In one embodiment, a single-domain antibody consists of all or a portion of the heavy chain variable domain of an antibody.

### 6. Anti-CTLA4 Binding Protein: Antibody Variants

In some embodiments, amino acid sequence modification(s) of the antibodies described herein are contemplated. For example, it may be desirable to improve the binding affinity and/or other biological properties of the antibody. Amino acid sequence variants of the antibody may be prepared by introducing appropriate changes into the nucleotide sequence encoding the antibody, or by peptide synthesis. Such modifications include, for example, deletions from, and/or insertions into and/or substitutions of, residues within the amino acid sequences of the antibody. Any combination of deletion, insertion, and substitution can be made to arrive at the final construct, provided that the final construct possesses the desired characteristics. The amino acid alterations may be introduced in the subject antibody amino acid sequence at the time that sequence is made.

A useful method for identification of certain residues or regions of the antibody that are preferred locations for mutagenesis is called “alanine scanning mutagenesis” as described by Cunningham and Wells (1989) Science, 244:1081-1085. Here, a residue or group of target residues are identified (e.g., charged residues such as Arg, Asp, His, Lys, and Glu) and replaced by a neutral or negatively charged amino acid (e.g., alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, Ala scanning or random mutagenesis is conducted at the target codon or region and the expressed immunoglobulins are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme or a polypeptide which increases the serum half-life of the antibody.

In some embodiments, FcRn mutations that improve pharmacokinetics include, but are not limited to, M428L, T250Q/M428L, M252Y/S254/T256E, P257I/N434H, D376V/N434H, P257U/Q311I, N434A, N434W, M428L/N434S, V2591/V308F, M252Y/S254/T256E, V2591/V308F/M428L, T307Q/N434A, T307Q/N434S, T307Q/E380A/N434A, V308P/N434A, N434H, V308P. In some embodiments, such mutations enhance antibody binding to FcRn at low pH but do not change the antibody affinity at neutral pH.

In certain embodiments, an antibody of the invention is altered to increase or decrease the extent to which the antibody is glycosylated. Glycosylation of polypeptides is typically either N-linked or O-linked, N-linked refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition or deletion of glycosylation sites to the antibody is conveniently accomplished by altering the amino acid sequence such that one or more of the above-described tripeptide sequences (for N-linked glycosylation sites) is created or removed. The alteration may also be made by the addition, deletion, or substitution of one or more serine or threonine residues to the sequence of the original antibody (for O-linked glycosylation sites).

Where the antibody comprises an Fc region, the carbohydrate attached thereto may be altered. For example, antibodies with a mature carbohydrate structure that lacks fucose attached to an Fc region of the antibody are described in US Pat Appl No US 2003/0157108 (Presta, L). See also US 2004/0093621 (Kyowa Hakko Kogyo Co., Ltd). Antibodies with a bisecting N-acetylglucosamine (GlcNAc) in the carbohydrate attached to an Fc region of the antibody are referenced in WO 2003/011878, Jean-Mairet et al, and U.S. Pat. No. 6,602,684, Umana et al. Antibodies with at least one galactose residue in the oligosaccharide attached to an Fc region of the antibody are reported in WO 1997/30087, Patel et al. See, also, WO 1998/58964 (Raju, S.) and WO 1999/22764 (Raju, S.) concerning antibodies with altered carbohydrate attached to the Fc region thereof. See also US 2005/0123546 (Umana et al.) on antigen-binding molecules with modified glycosylation.

In certain embodiments, a glycosylation variant comprises an Fc region, wherein a carbohydrate structure attached to the Fc region lacks fucose or has reduced fucose. Such variants have improved ADCC function. Optionally, the Fc region further comprises one or more amino acid substitutions therein which further improve ADCC, for example, substitutions at positions 298, 333, and/or 334 of the Fc region (EU numbering of residues). Examples of publications related to “afucosylated,” “defucosylated” or “fucose-deficient” antibodies include: US 2003/0157108; WO 2000/61739; WO 2001/29246; US 200300115614; US 2002/0164328; US 2004/0093621; US 2004/0132140; US 2004/0110704; US 2004/0110282: US 2004/0109865; WO 2003/085119; WO 2003/084570; WO 2005/035586; WO 2005/035778; WO2005/053742; Okazaki et al. J. Mol. Biol. 336:1239-1249 (2004); Yamane-Ohnuki et al. Biotech. Bioeng. 87: 614 (2004). Examples of cell lines producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (Yamane-Ohnuki et al. Biotech. Bioeng. 87: 614 (2004)), and cells overexpressing β1,4-N-acetylglycosminyltransferase III (GnT-III) and Golgi μ-mannosidase II (ManII).

In any of the embodiments herein, the anti-CTLA4 binding proteins can be engineered to improve antibody-dependent cell-mediated cytotoxicity (ADCC) activity. In some embodiments, the anti-CTLA4 binding protein may be produced in a cell line having an alpha1,6-fucosyltransferase (Fut8) knockout. In some further embodiments, the anti-CTLA4 binding protein may be produced in a cell line overexpressing β1,4-N-acetylglycosminyltransferase III (GnT-III). In further embodiments, the cell line additionally overexpresses Golgi μ-mannosidase II (ManII). In some of the embodiments herein, the anti-CTLA4 binding protein may comprise at least one amino acid substitution in the Fc region that improves ADCC activity.

In one embodiment, the antibody is altered to improve its serum half-life. To increase the serum half-life of the antibody, one may incorporate a FcRN/salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule (US 2003/0190311, U.S. Pat. No. 6,821,505: U.S. Pat. Nos. 6,165,745; 5,624,821; 5,648,260; 6,165,745; 5,834,597).

Another type of variant is an amino acid substitution variant. These variants have at least one amino acid residue in the antibody molecule replaced by a different residue. Sites of interest for substitutional mutagenesis include the hypervariable regions, but FR alterations are also contemplated. Conservative substitutions are shown in Table 1 under the heading of “preferred substitutions.” If such substitutions result in a desirable change in biological activity, then more substantial changes, denominated “exemplary substitutions” in Table 1, or as further described below in reference to amino acid classes, may be introduced and the products screened.

TABLE 1
Amino Acid Substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Substantial modifications in the biological properties of the antibody are accomplished by selecting substitutions that differ significantly in their effect on maintaining(a) the structure of the polypeptide backbone in the area of the substitution, for example, as a sheet or helical conformation, (b) the charge or hydrophobicity of the molecule at the target site, or c) the bulk of the side chain. Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (I), Pro (P), Phe (F), Trp (W), Met (M)
(2) uncharged polar: Gly (G), Ser (S), Thr (T), Cys (C), Tyr (Y), Asn (N), Gin (Q)
(3) acidic: Asp (D), Glu (E)
(4) basic: Lys (K), Arg (R), His (H)

Alternatively, naturally occurring residues may be divided into groups based on common side-chain properties:

(1) hydrophobic: Norleucine, Met, Ala, Val, Leu, lie;
(2) neutral hydrophilic: Cys, Ser, Thr. Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class. Such substituted residues also may be introduced into the conservative substitution sites or, into the remaining (non-conserved) sites.

One type of substitutional variant involves substituting one or more hypervariable region residues of a parent antibody (e.g., a humanized or human antibody). Generally, the resulting variant(s) selected for further development will have modified (e.g., improved) biological properties relative to the parent antibody from which they are generated. A convenient way for generating such substitutional variants involves affinity maturation using phage display. Briefly, several hypervariable region sites (e.g., 6-7 sites) are mutated to generate all possible amino acid substitutions at each site. The antibodies thus generated are displayed from filamentous phage particles as fusions to at least part of a phage coat protein (e.g., the gene III product of M13) packaged within each particle. The phage-displayed variants are then screened for their biological activity (e.g., binding affinity). In order to identify candidate hypervariable region sites for modification, scanning mutagenesis (e.g., alanine scanning) can be performed to identify hypervariable region residues contributing significantly to antigen binding. Alternatively, or additionally, it may be beneficial to analyze a crystal structure of the antigen-antibody complex to identify contact points between the antibody and antigen. Such contact residues and neighboring residues are candidates for substitution according to techniques known in the art, including those elaborated herein. Once such variants are generated, the panel of variants is subjected to screening using techniques known in the art, including those described herein, and antibodies with superior properties in one or more relevant assays may be selected for further development.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

It may be desirable to introduce one or more amino acid modifications in an Fc region of antibodies of the invention, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions including that of a hinge cysteine.

In some embodiments, an anti-CTLA4 binding protein (e.g., anti-CTLA4 antibody or antigen-binding fragment thereof, or anti-CTLA4 bispecific antibody) provided herein has an IgG1 isotype with enhanced effector function. In some embodiments, the anti-CTLA4 antibody or antigen-binding fragment thereof is afucosylated. In some embodiments, the anti-CTLA4 bispecific antibody is afucosylated. In some embodiments, the anti-CTLA4 antibody or antigen-binding fragment thereof has increased levels of mannose moieties. In some embodiments, the anti-CTLA4 antibody or antigen-binding fragment thereof has increased levels of bisecting glycan moieties. In some embodiments, the anti-CTLA4 bispecific antibody has increased levels of mannose moieties. In some embodiments, the IgG1 comprises amino acid mutations.

In some embodiments, an anti-CTLA4 antibody or antigen-binding fragment thereof, or anti-CTLA4 bispecific antibody provided herein has an IgG1 isotype (e.g., a human IgG1 isotype). In one embodiment, the IgG1 comprises the amino acid substitutions S298A, E333A, and K334A wherein the amino acid residues are numbered according to the EU index as in Kabat. In one embodiment, the IgG1 comprises the amino acid substitutions S239D and I332E wherein the amino acid residues are numbered according to the EU index as in Kabat. In one embodiment, the IgG1 comprises the amino acid substitutions S239D, A330L, and I332E wherein the amino acid residues are numbered according to the EU index as in Kabat. In one embodiment, the IgG1 comprises the amino acid substitutions P247I and A339D or A339Q wherein the amino acid residues are numbered according to the EU index as in Kabat. In one embodiment, the IgG1 comprises the amino acid substitutions D280H, K290S with or without S298D or S298V wherein the amino acid residues are numbered according to the EU index as in Kabat. In one embodiment, the IgG1 comprises the amino acid substitutions F243L, R292P, and Y300L wherein the amino acid residues are numbered according to the EU index as in Kabat. In one embodiment, the IgG1 comprises the amino acid substitutions F243L, R292P, Y300L, and P396L wherein the amino acid residues are numbered according to the EU index as in Kabat. In one embodiment, the IgG1 comprises the amino acid substitutions F243L, R292P, Y300L, V305I, and P396L wherein the amino acid residues are numbered according to the EU index as in Kabat. In one embodiment, the IgG1 comprises the amino acid substitutions G236A, S239D, and I332E wherein the amino acid residues are numbered according to the EU index as in Kabat. In one embodiment, the IgG1 comprises the amino acid substitutions K326A and E333A wherein the amino acid residues are numbered according to the EU index as in Kabat. In one embodiment, the IgG1 comprises the amino acid substitutions K326W and E333S wherein the amino acid residues are numbered according to the EU index as in Kabat. In one embodiment, the IgG1 comprises the amino acid substitutions K290E or K290N, S298G, T299A, and/or K326E wherein the amino acid residues are numbered according to the EU index as in Kabat. In some aspects, exemplary heavy chain constant region comprised in the provided CTLA4 binding proteins, such as antibodies or antigen-binding fragments thereof, include the heavy chain constant region sequence set forth in SEQ ID NO: 35-38. In some aspects, exemplary light chain constant region comprised in the provided CTLA4 binding proteins, such as antibodies or antigen-binding fragments thereof, include the light chain constant region sequence set forth in SEQ ID NO: 39.

In some aspects, the provided CTLA4 binding proteins, such as antibodies or antigen-binding fragments thereof, include a heavy chain constant region comprising the sequence set forth in SEQ ID NO:38. In some aspects, the provided CTLA4 binding proteins, such as antibodies or antigen-binding fragments thereof, include a heavy chain constant region comprising the sequence set forth in SEQ ID NO:35. In some aspects, the provided CTLA4 binding proteins, such as antibodies or antigen-binding fragments thereof, include a light chain constant region comprising the sequence set forth in SEQ ID NO:39.

In some aspects, the provided CTLA4 binding proteins, such as antibodies or antigen-binding fragments thereof, include a heavy chain constant region comprising the sequence set forth in SEQ ID NO:38 and a light chain constant region comprising the sequence set forth in SEQ ID NO:39. In some aspects, the provided CTLA4 binding proteins, such as antibodies or antigen-binding fragments thereof, include a heavy chain constant region comprising the sequence set forth in SEQ ID NO:35 and a light chain constant region comprising the sequence set forth in SEQ ID NO:39.

In accordance with this description and the teachings of the art, it is contemplated that in some embodiments, an antibody of the invention may comprise one or more alterations as compared to the wild type counterpart antibody, e.g. in the Fc region. These antibodies would nonetheless retain substantially the same characteristics required for therapeutic utility as compared to their wild type counterpart. For example, it is thought that certain alterations can be made in the Fc region that would result in altered (i.e., either improved or diminished) C1q binding and/or Complement Dependent Cytotoxicity (CDC), e.g., as described in WO99/51642. See also Duncan & Winter Nature 322:738-40 (1988); U.S. Pat. Nos. 5,648,260; 5,624,821; and WO94/29351 concerning other examples of Fc region variants, WO00/42072 (Presta) and WO 2004/056312 (Lowman) describe antibody variants with improved or diminished binding to FcRs. The content of these patent publications are specifically incorporated herein by reference. See, also, Shields et al. J. Biol. Chem. 9(2): 6591-6604 (2001). Antibodies with increased half-lives and improved binding to the neonatal Fe receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). These antibodies comprise an Fc region with one or more substitutions therein which improve binding of the Fc region to FcRn. Polypeptide variants with altered Fc region amino acid sequences and increased or decreased C1q binding capability are described in U.S. Pat. No. 6,194,551BI, WO99/51642. The contents of those patent publications are specifically incorporated herein by reference. See, also, Idusogie et al. J. Immunol. 164:4178-4184 (2000).

### 7. Antibody-Drug Conjugates

The invention also provides antibody-drug conjugates (ADCs) comprising an anti-CTLA4 binding protein provided herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, the one or more drugs conjugated to the antibody-drug conjugate, includes but is not limited to a maytansinoid (sec U.S. Pat. Nos. 5,208,020, 5,416,064 and European Patent EP 0 425 235 BI); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment the one or more drugs conjugated to the antibody-drug conjugate, includes but is not limited to an inhibitor of tubulin polymerization (e.g., maytansinoids and auristatins), DNA damaging agents (e.g., pyrrolobenzodiazepine (PBD) dimers, calicheamicins, duocarmycins and indo-linobenzodiazepine dimers), and DNA synthesis inhibitors (e.g., exatecan derivative Dxd).

In another embodiment, an antibody-drug conjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins. Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another embodiment, an antibody-drug conjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc^{99m} or I¹²³, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron.

Conjugates of an anti-CTLA4 binding protein (e.g., anti-CTLA4 antibody or antigen-binding fragment thereof) and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine). bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled I-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SlAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A.).

### 8. Vectors, Host Cells, and Recombinant Methods

For recombinant production of an anti-CTLA4 binding proteins of the invention, the nucleic acid encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the heavy and light chains of the antibody). Many vectors are available. The choice of vector depends in part on the host cell to be used. Generally, host cells are of either prokaryotic or eukaryotic (generally mammalian) origin. It will be appreciated that constant regions of any isotype can be used for this purpose, including IgG, IgM, IgA, IgD, and IgE constant regions, and that such constant regions can be obtained from any human or animal species.

### 9. Generating Binding Proteins Using Prokaryotic Host Cells

### a) Vector Construction

Polynucleotide sequences encoding polypeptide components of the anti-CTLA4 binding proteins of the invention can be obtained using standard recombinant techniques. Desired polynucleotide sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes-encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as λGEM™-11 may be utilized in making a recombinant vector which can be used to transform susceptible host cells such as E. coli LE392.

The expression vector of the invention may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the invention. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the β-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al. (1980) Cell 20:269) using linkers or adaptors to supply any required restriction sites.

In one aspect of the invention, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, lpp, or heat-stable enterotoxin II (STII) leaders, LamB, PhoE, PelB, OmpA and MBP. In one embodiment of the invention, the signal sequences used in both cistrons of the expression system are STII signal sequences or variants thereof.

In another aspect, the production of the immunoglobulins according to the invention can occur in the cytoplasm of the host cell, and therefore does not require the presence of secretion signal sequences within each cistron. In that regard, immunoglobulin light and heavy chains are expressed with or without the sequences for the additional molecule, etc., folded and assembled to form functional immunoglobulins within the cytoplasm. Certain host strains (e.g., the E. coli trxB-strains) provide cytoplasm conditions that are favorable for disulfide bond formation, thereby permitting proper folding and assembly of expressed protein subunits. Proba and Pluckthun Gene, 159:203 (1995).

Anti-CTLA4 binding proteins of the invention can also be produced by using an expression system in which the quantitative ratio of expressed polypeptide components can be modulated in order to maximize the yield of secreted and properly assembled antibodies of the invention. Such modulation is accomplished at least in part by simultaneously modulating translational strengths for the polypeptide components.

Prokaryotic host cells suitable for expressing anti-CTLA4 binding protein of the invention include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In one embodiment, gram-negative cells are used. In one embodiment, E. coli cells are used as hosts for the invention. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 ΔfhuA (ΔtonA) ptr3 lac Iq lacL8 ΔompTA(nmpc-fepE) degP41 kanR (U.S. Pat. No. 5,639,635). Other strains and derivatives thereof, such as E. coli 294 (ATCC 31,446), E. coli B, E. coliλ 1776 (ATCC 31,537) and E. coli RV308(ATCC 31,608) are also suitable. These examples are illustrative rather than limiting. Methods for constructing derivatives of any of the above-mentioned bacteria having defined genotypes are known in the art and described in, for example, Bass et al., Proteins. 8:309-314 (1990). It is generally necessary to select the appropriate bacteria taking into consideration replicability of the replicon in the cells of a bacterium. For example, E. coli, Serratia, or Salmonella species can be suitably used as the host when well-known plasmids such as pBR322, pBR325, pACYC177, or pKN410 are used to supply the replicon. Typically the host cell should secrete minimal amounts of proteolytic enzymes, and additional protease inhibitors may desirably be incorporated in the cell culture.

### b) Binding Protein Production

Host cells are transformed with the above-described expression vectors and cultured in conventional nutrient media modified as appropriate for inducing promoters, selecting transformants, or amplifying the genes encoding the desired sequences.

Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the anti-CTLA4 binding proteins of the invention are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. In certain embodiments, for E. coli growth, growth temperatures range from about 20° C. to about 39° C.; from about 25° C. to about 37° C.; or about 30° C. The pH of the medium may be any pH ranging from about 5 to about 9, depending mainly on the host organism. In certain embodiments, for E. coli, the pH is from about 6.8 to about 7.4, or about 7.0.

If an inducible promoter is used in the expression vector of the invention, protein expression is induced under conditions suitable for the activation of the promoter. In one aspect of the invention, PhoA promoters are used for controlling transcription of the polypeptides. Accordingly, the transformed host cells are cultured in a phosphate-limiting medium for induction. In certain embodiments, the phosphate-limiting medium is the C. R. A.P. medium (see, e.g., Simmons et al., J. Immunol. Methods (2002), 263:133-147). A variety of other inducers may be used, according to the vector construct employed, as is known in the art.

In one embodiment, the expressed anti-CTLA4 binding proteins of the present invention are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

In one aspect of the invention, anti-CTLA4 binding protein production is conducted in large quantity by a fermentation process. Various large-scale fed-batch fermentation procedures are available for production of recombinant proteins. Large-scale fermentations have at least 1000 liters of capacity, and in certain embodiments, about 1,000 to 100,000 liters of capacity. These fermenters use agitator impellers to distribute oxygen and nutrients, especially glucose. Small scale fermentation refers generally to fermentation in a fermenter that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD550 of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the polypeptides of the invention, various fermentation conditions can be modified. For example, to improve the proper assembly and folding of the secreted antibody polypeptides, additional vectors overexpressing chaperone proteins, such as Dsb proteins (DsbA, DsbB, DsbC, DsbD and or DsbG) or FkpA (a peptidylprolyl cis,trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J. Biol. Chem. 274:19601-19605; Georgiou et al., U.S. Pat. No. 6,083,715; Georgiou et al., U.S. Pat. No. 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210.

To minimize proteolysis of expressed heterologous proteins (especially those that are proteolytically sensitive), certain host strains deficient for proteolytic enzymes can be used for the present invention. For example, host cell strains may be modified to effect genetic mutation(s) in the genes encoding known bacterial proteases such as Protease III, OmpT, DegP, Tsp, Protease 1, Protease Mi, Protease V, Protease VI and combinations thereof. Some E. coli protease-deficient strains are available and described in, for example, Joly et al. (1998), supra; Georgiou et al., U.S. Pat. No. 5,264,365; Georgiou et al., U.S. Pat. No. 5,508,192; Hara et al., Microbial Drug Resistance, 2:63-72 (1996).

In one embodiment, E. coli strains deficient for proteolytic enzymes and transformed with plasmids overexpressing one or more chaperone proteins are used as host cells in the expression system of the invention.

### c) Binding Protein Purification

In one embodiment, the antibody protein produced herein is further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

In one aspect, Protein A immobilized on a solid phase is used for immunoaffinity purification of the antibody products of the invention. Protein A is a 41 kD cell wall protein from Staphylococcus aureus which binds with a high affinity to the Fc region of antibodies. Lindmark et al (1983) J. Immunol. Meth. 62:1-13. The solid phase to which Protein A is immobilized can be a column comprising a glass or silica surface, or a controlled pore glass column or a silicic acid column. In some applications, the column is coated with a reagent, such as glycerol, to possibly prevent nonspecific adherence of contaminants.

As the first step of purification, a preparation derived from the cell culture as described above can be applied onto a Protein A immobilized solid phase to allow specific binding of the antibody of interest to Protein A. The solid phase would then be washed to remove contaminants non-specifically bound to the solid phase. Finally the antibody of interest is recovered from the solid phase by elution.

### 10. Generating Binding Proteins Using Eukaryotic Host Cells

A vector for use in a eukaryotic host cell generally includes one or more of the following non-limiting components: a signal sequence, an origin of replication, one or more marker genes, an enhancer element, a promoter, and a transcription termination sequence.

### a) Signal Sequence Component

A vector for use in a eukaryotic host cell may also contain a signal sequence or other polypeptide having a specific cleavage site at the N-terminus of the mature protein or polypeptide of interest. The heterologous signal sequence selected may be one that is recognized and processed (i.e., cleaved by a signal peptidase) by the host cell. In mammalian cell expression, mammalian signal sequences as well as viral secretory leaders, for example, the herpes simplex gD signal, are available. The DNA for such a precursor region is ligated in reading frame to DNA encoding the antibody.

### b) Origin of Replication

Generally, an origin of replication component is not needed for mammalian expression vectors. For example, the SV40 origin may typically be used only because it contains the early promoter.

### c) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, where relevant, or (c) supply critical nutrients not available from complex media.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the anti-CTLA4 binding protein encoding nucleic acid, such as DHFR, thymidine kinase, metallothionein-I and -II, primate metallothionein genes, adenosine deaminase, ornithine decarboxylase, etc.

For example, in some embodiments, cells transformed with the DHFR selection gene are first identified by culturing all of the transformants in a culture medium that contains methotrexate (Mtx), a competitive antagonist of DHFR. In some embodiments, an appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with DNA sequences encoding an anti-CTLA4 binding protein, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199. Host cells may include NS0, including cell lines deficient in glutamine synthetase (GS). Methods for the use of GS as a selectable marker for mammalian cells are described in U.S. Pat. Nos. 5,122,464 and 5,891,693.

### d) Promoter Component

Expression and cloning vectors usually contain a promoter that is recognized by the host organism and is operably linked to nucleic acid encoding an anti-CTLA4 binding protein of interest. Promoter sequences are known for eukaryotes. For example, virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the Y end of most eukaryotic genes is an AATAAA sequence that may be the signal for addition of the poly A tail to the 3′ end of the coding sequence. In certain embodiments, any or all of these sequences may be suitably inserted into eukaryotic expression vectors.

Transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982), describing expression of human 1-interferon cDNA in murine cells under the control of a thymidine kinase promoter from herpes simplex virus. Alternatively, the Rous Sarcoma Virus long terminal repeat can be used as the promoter.

### e) Enhancer Element Component

of DNA encoding an antibody of this invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the human cytomegalovirus early promoter enhancer, the murine cytomegalovirus early promoter enhancer, the polyoma enhancer on the late side of the replication origin, and adenovirus enhancers. See also Yaniv, Nature 297:17-18 (1982) describing enhancer elements for activation of eukaryotic promoters. The enhancer may be spliced into the vector at a position 5′ or 3′ to the antibody polypeptide-encoding sequence, but is generally located at a site 5′ from the promoter.

### f) Transcription Termination Component

Expression vectors used in eukaryotic host cells may also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding an antibody. One useful transcription termination component is the bovine growth hormone polyadenylation region. See W094/l 1026 and the expression vector disclosed therein.

### g) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); murine sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); murine mammary tumor (MMT 060562, ATCC CCL51); TRI cells (Mather et al.. Annals N.Y. Acad. Sci. 383:44-68 (1982)); MRC 5 cells; FS4 cells; and a human hepatoma line (Hep G2).

Host cells am transformed with the above-described-expression or cloning vectors for anti-CTLA4 binding protein production and cultured in conventional nutrient media modified as appropriate for inducing promoters, selecting transformants, or amplifying the genes encoding the desired sequences.

### h) Culturing the Host Cells

The host cells used to produce anti-CTLA4 binding proteins of this invention may be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560.655; or 5,122,469; WO 9003430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other supplements may also be included at appropriate concentrations that would be known to those skilled in the art. The culture conditions, such as temperature, pH, and the like, are those previously used with the host cell selected for expression, and will be apparent to the ordinarily skilled artisan.

### i) Purification of Binding Protein

When using recombinant techniques, the anti-CTLA4 binding proteins can be produced intracellularly, or directly secreted into the medium. If the antibody is produced intracellularly, as a first step, the particulate debris, either host cells or lysed fragments, may be removed, for example, by centrifugation or ultrafiltration. Where the anti-CTLA4 binding protein is secreted into the medium, supernatants from such expression systems may be first concentrated using a commercially available protein concentration filter, for example, an Amicon or Millipore Pellicon ultrafiltration unit. A protease inhibitor such as PMSF may be included in any of the foregoing steps to inhibit proteolysis, and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being a convenient technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Methods 62:1-13 (1983)). Protein G is recommended for all murine isotypes and for human γ3 (Guss et al., EMBO J. 5:15671575 (1986)). The matrix to which the affinity ligand is attached may be agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation arm also available depending on the antibody to be recovered.

Following any preliminary purification step(s), the mixture comprising the binding protein of interest and contaminants may be subjected to further purification, for example, by low pH hydrophobic interaction chromatography using an elution buffer at a pH between about 2.5-4.5, performed at low salt concentrations (e.g., from about 0-0.25M salt).

In general, various methodologies for preparing antibodies for use in research, testing, and clinical use are well-established in the art, consistent with the above-described methodologies and/or as deemed appropriate by one skilled in the art for a particular antibody of interest.

### IV. Pd1 Programmed Death 1(PD-1)

Programmed Death 1 (PD-1) (also known as Programmed Cell Death 1) is a type I transmembrane protein of 268 amino acids originally identified by subtractive hybridization of a mouse T cell line undergoing apoptosis (Ishida et al., Embo J., 11: 3887-95 (1992)). PD-1 is a member of the CD28/CTLA-4 family of T-cell regulators, and is expressed on activated T-cells, B-cells, and myeloid lineage cells (Greenwald et al., Annu. Rev. Immunol., 23: 515-548 (2005); and Sharpe et al., Nat. Immunol., 8: 239-245 (2007)). PD-1 is an inhibitory member of the CD28 family of receptors, that also includes CD28, CTLA-4, ICOS and BTLA, PD-1 is expressed on activated B cells, T cells, and myeloid cells (Agata et al., supra; Okazaki et al. (2002) Curr. Opin. Immunol 14:391779-82; Bennett et al. (2003) J Immunol 170:711-8).

Two ligands for PD-1 have been identified, PD ligand 1 (PD-L1) and PD ligand 2 (PD-L2), both of which belong to the B7 protein superfamily (Greenwald et al. supra). PD-L1 is expressed in a variety of cell types, including cells of the lung, heart, thymus, spleen, and kidney (see, e.g., Freeman et al., J. Exp. Med., 192(7): 1027-1034 (2000); and Yamazaki et al, J. Immunol., 169(10): 5538-5545 (2002)). PD-L1 expression is upregulated on macrophages and dendritic cells (DCs) in response to lipopolysaccharide (LPS) and GM-CSF treatment, and on T-cells and B-cells upon signaling via T-cell and B-cell receptors. PD-L1 also is expressed in a variety of murine tumor cell lines (see, e.g., Iwai et al., Proc. Natl Acad. Sci. USA, 99(9): 12293-12297 (2002); and Blank et al., Cancer Res., 64(3): 1140-1145 (2004)). In contrast, PD-L2 exhibits a more restricted expression pattern and is expressed primarily by antigen presenting cells (e.g., dendritic cells and macrophages), and some tumor cell lines (see, e.g., Latchman et al., Nat. Immunol., 2(3): 261-238 (2001)). High PD-L1 expression in tumors, whether on the tumor cell, stroma, or other cells within the tumor microenvironment, correlates with poor clinical prognosis, presumably by inhibiting effector T cells and upregulating regulatory T cells (Treg) in the tumor.

PD-1 negatively regulates T-cell activation, and this inhibitory function is linked to an immunoreceptor tyrosine-based switch motif (ITSM) in the cytoplasmic domain (see, e.g., Greenwald et al., supra: and Parry et al., Mol. Cell. Biol., 25: 9543-9553 (2005)). PD-1 deficiency can lead to autoimmunity. For example, C57BL/6 PD-1 knockout mice have been shown to develop a lupus-like syndrome (see, e.g., Nishimura et al., Immunity. 11: 141-1151 (1999)). In humans, a single nucleotide polymorphism in the PD-1 gene is associated with higher incidences of systemic lupus erythematosus, type 1 diabetes, rheumatoid arthritis, and progression of multiple sclerosis (see, e.g., Nielsen et al., Tissue Antigens, 62(6): 492-497 (2003); Bertsias et al., Arthritis Rheum., 60(1): 207-218 (2009); Ni et al, Hum. Genet., 121(2): 223-232(2007); Tahoori et al., Clin. Exp. Rheumatol., 29(5): 763-767 (2011); and Kroner et al., Ann. Neurol., 58(1): 50-57 (2005)). Abnormal PD-1 expression also has been implicated in T-cell dysfunctions in several pathologies, such as tumor immune evasion and chronic viral infections (see, e.g., Barber et al., Nature, 439: 682-687 (2006); and Sharpe et al., supra).

Recent studies demonstrate that T-cell suppression induced by PD-1 also plays a role in the suppression of anti-tumor immunity. For example, PD-L1 is expressed on a variety of human and mouse tumors, and binding of PD-1 to PD-L1 on tumors results in T-cell suppression and tumor immune evasion and protection (Dong et al., Nat. Med., 8: 793-800 (2002)). Expression of PD-L1 by tumor cells has been directly associated with their resistance to lysis by anti-tumor T-cells in vitro (Dong et al., supra; and Blank et al., Cancer Res., 64: 1140-1145 (2004)). PD-1 knockout mice are resistant to tumor challenge (Iwai et al., Int. Immunol., 17: 133-144 (2005)), and T-cells from PD-1 knockout mice are highly effective in tumor rejection when adoptively transferred to tumor-bearing mice (Blank et al., supra). Blocking PD-1 inhibitory signals using a monoclonal antibody can potentiate host anti-tumor immunity in mice (Iwai et al., supra; and Hirano et al. Cancer Res., 65: 1089-1096 (2005)), and high levels of PD-L1 expression in tumors are associated with poor prognosis for many human cancer types (Hamanishi et al., Proc. Natl. Acad. Sci. USA. 104: 3360-335 (2007), Brown et al, J. Immunol., 170:1257-1266 (2003); and Flies et al., Yale Journal of Biology and Medicine, 84(4): 409-421 (2011)).

In view of the foregoing, strategies for inhibiting PD-1 activity to treat various types of cancer and for immunopotentiation (e.g., to treat infectious diseases) have been developed (see, e.g., Ascierto et al., Clin. Cancer. Res., 19(5): 1009-1020 (2013)). In this respect, monoclonal antibodies targeting PD-1 have been developed for the treatment of cancer (see, e.g., Weber, Semin. Oncol., 37(5): 430-4309 (2010); and Tang et al., Current Oncology Reports, 15(2): 98-104 (2013)). For example, nivolumab (also known as BMS-936558) produced complete or partial responses in non-small-cell lung cancer, melanoma, and renal-cell cancer in a Phase I clinical trial (see, e.g., Topalian, New England. Med. 366: 2443-2454 (2012)), and is currently in Phase III clinical trials. MK-3575 is a humanized monoclonal antibody directed against PD-1 that has shown evidence of antitumor activity in Phase I clinical trials (see, e.g., Patnaik et al., 2012 American Society of Clinical Oncology (ASCO) Annual Meeting, Abstract #2512). In addition, recent evidence suggests that therapies which target PD-1 may enhance immune responses against pathogens, such as HIV (see, e.g., Porichis et al., Curr. HIV/AIDS Rep., 9(1): 81-90 (2012)). Despite these advances, however, the efficacy of these potential therapies in humans may be limited.

Agents that Inhibit PD-1 Signaling

The present disclosure provides methods of treating cancer that include administering compositions that deliver programmed death-1 protein (PD-1) signaling agents according to regimens that may achieve clinical benefit(s).

Agents that inhibit PD-1 signaling for use in therapies of the present disclosure include those that bind to and block PD-1 receptors on T cells without triggering inhibitory signal transduction, agents that bind to PD-1 ligands to prevent their binding to PD-1, agents that do both, and agents that prevent expression of genes that encode either PD-1 or natural ligands of PD-1. Compounds that bind to natural ligands of PD-1 include PD-1 itself, as well as active fragments of PD-1, and in the case of the B7-H1 ligand, B7.1 proteins and fragments. Such antagonists include proteins, antibodies, anti-sense molecules and small organics.

The present disclosure describes, at least in part, PD-1 agents (e.g., PD-1 agents or PD-L1 agents) and various compositions and methods relating thereto. In some embodiments, a PD-1 signaling agent (e.g., anti-PD-1 antibody agent) binds an epitope of PD-1 which blocks the binding of PD-1 to any one or more of its putative ligands.

In some embodiments, an agent that inhibits PD-1 signaling agent is for use in combination therapies of the present disclosure is an antibody agent. In some embodiments, a PD-1 antibody agent binds an epitope of PD-1 which blocks the binding of PD-1 to any one or more of its putative ligands. In some embodiments, a PD-1 antibody agent binds an epitope of PD-1 which blocks the binding of PD-1 to two or more of its putative ligands. In embodiments, a PD-1 antibody agent binds an epitope of a PD-1 protein which blocks the binding of PD-1 to PD-L1 and/or PD-L2. PD-1 antibody agents of the present disclosure may comprise a heavy chain constant region (Fc) of any suitable class. In some embodiments, a PD-1 antibody agent comprises a heavy chain constant region that is based upon wild-type IgG1, IgG2, or IgG4 antibodies, or variants thereof.

In some embodiments, an agent that inhibits PD-1 signaling is a monoclonal antibody, or a fragment thereof. In some embodiments, an antibody agent that inhibits PD-1 signaling is a PD-1 antibody or fragment thereof. Monoclonal antibodies that target PD-1 that have been tested in clinical studies and/or received marketing approval in the United States. Examples of antibody agents that target PD-1 signaling include, for example, any of the antibody agents listed in the following Table 2:

TABLE 2
Anti-PD-1 antibody agents
Antibody Agent Target (Format)

Opdivo Nivolumab
PD-1 (Human IgG4)
Keytruda Pembrolizumab
PD-1 (Humanized IgG4)
Tecentriq Atezolizumab
PD-L1 (Human IgG1)
Imfinzi Durvalumab
PD-L1 (Human IgG1)
Bavencio Avelumab
PD-L1 (Human IgG1)
PDR001
PD-1 (Humanized IgG4)
Cemiplimab
PD-1 (fully human IgG4)
BGB-A317
PD-1 (Humanized IgG4) engineered
to not bind FcγRI
LY3300054
PD-L1
BI 754091
(anti-PD-1)
IBI308
(anti-PD-1)
INCSHR-1210
(anti-PD-1)
JNJ-63723283
(anti-PD-1)
JS-001
(anti-PD-1)
MEDI0680 (AMP-514)
anti-PD-1 (Humanized IgG4)
MGA-012
(anti-PD-1)
PF-06801591
(anti-PD-1)
Dostarlimab (TSR-042)
anti-PD-1 (Humanized IgG4)
CX-072
anti-PD-L1
FAZ053
anti-PD-L1
PD-L1 millamolecule

In some embodiments, the PD-1 agent is selected from a PD-1 agent provided in Table 2.

In some embodiments, an antibody agent that inhibits PD-1 signaling is atezolizumab, avelumab, BGB-A317, BI 754091, CX-072, durvalumab, FAZ053, IBI308, INCSHR-1210, JNJ-63723283, JS-001, MEDI-0680, MGA-012, nivolumab, PDR001, pembrolizumab, PF-06801591, cemiplimab, dostarlimab, any of the antibodies disclosed in WO2014/179664, or derivatives thereof. In some embodiments, an antibody agent that inhibits PD-1 signaling is a PD-1 antibody selected from the group consisting of BGB-A317, BI 754091, CX-072, FAZ053, IBI308, INCSHR-1210, JNJ-63723283, JS-001, LY3300054, MEDI-0680, MGA-012, nivolumab, PD-L1 millamolecule, PDR001, pembrolizumab, PF-06801591, cemiplimab, and dostarlimab. In some embodiments, an antibody agent that inhibits PD-1 signaling is a PD-1 antibody selected from the group consisting of nivolumab, pembrolizumab, and dostarlimab.

In some embodiments, a PD-1 signaling agent is pembrolizumab, nivolumab, atezolizumab, durvalumab, avelumab, dostarlimab, PDR-001, tislelizumab (BGB-A317), cemiplimab (REGN2810). LY-3300054, JNJ-63723283, MGA012, BI-754091, IBI-308, camrelizumab (HR-301210). BCD-100, JS-001, CX-072, BGB-A333, AMP-514 (MEDI-0680), AGEN-2034, CS1001, Sym-021, SHR-1316, PF-06801591, LZM009, KN-035, AB122, genolimzumab (CBT-501), FAZ-053, CK-301, AK 104, or GLS-010, or any of the PD-1 antibodies disclosed in WO2014/179664. In embodiments, an immune checkpoint inhibitor is a PD-1 inhibitor. In embodiments, a PD-1 inhibitor is a PD-1 signaling agent (e.g., an antibody, an antibody conjugate, or an antigen-binding fragment thereof). In embodiments, a PD-1 inhibitor is a PD-L1 or PD-L2 binding agent is durvalumab, atezolizumab, avelumab, BGB-A333, SHR-1316, FAZ-053, CK-301, or, PD-L1 millamolecule, or derivatives thereof.

In some embodiments, a PD-1-binding agent (e.g., anti-PD-1 antibody agent) binds an epitope of PD-1 which blocks the binding of PD-1 to two or more of its putative ligands. In some embodiments, a PD-1-binding agent (e.g., anti-PD-1 antibody agent) binds an epitope of a PD-1 protein which blocks the binding of PD-1 to PD-L1 and/or PD-L2, PD-1-binding agents (e.g., anti-PD-1 antibody agents) of the present disclosure may comprise a heavy chain constant region (Fc) of any suitable class. In some embodiments, a PD-1-binding agent (e.g., anti-PD-1 antibody agent) comprises a heavy chain constant region that is based upon wild-type IgG1, IgG2, or IgG4 antibodies, or variants thereof. In some embodiments, a PD-1-binding agent is a monoclonal antibody.

In some embodiments a PD-1-binding agent is or comprises an immunoglobulin G4 (IgG4) humanized monoclonal antibody (mAb). In some embodiments, a PD-1-binding agent comprises a human IGHG4*01 polypeptide. In some embodiments, a PD-1-binding agent comprises one or more mutations within the IgG heavy chain region. In some embodiments, a PD-1-binding agent comprises an IgG4 heavy chain constant region having one or more mutations in the heavy chain constant region. In some embodiments, a PD-1-binding agent comprises an IgG4 heavy chain constant region having one or more mutations in hinge region. It is envisioned that in some embodiments, a mutation in the IgG4 hinge region may prevent half molecule exchange with other IgG4 molecules. In some embodiments, the one or more mutations in hinge region of IgG4 may include a serine to proline stabilizing mutation that prevents half molecule exchange with other IgG4 molecules. In some embodiments, the one or more mutations in hinge region of IgG4 may include an S228P mutation. See, e.g., J. Biol. Chem. 2015; 290(9):5462-5469. Without wishing to be bound by theory, it is envisioned that this point mutation serves to stabilize the hinge of the antibody heavy chain.

In some embodiments, a PD-1-binding agent is nivolumab, pembrolizumab, atezolizumab, durvalumab, avelumab, or any of the antibodies disclosed in WO2014/179664.

Pembrolizumab is an anti-PD-1 monoclonal antibody (“mAb”) (also known as MK-3475, SCH 9000475, Keytruda). Pembrolizumab is an immunoglobulin G4/kappa isotype humanized mAb. The mechanism of pembrolizumab consists of the mAb binding to the PD-1 receptor of lymphocytes to block the interaction of PD-1 with PD-L1 and PD-L2 ligands produced by other cells in the body, including tumor cells of certain cancers.

Similarly to pembrolizumab, nivolumab (also known as BMS-936558, Opdivo) was first approved by the FDA in 2014 to treat melanoma that cannot be surgically removed or has metastasized following treatment with ipilimumab and a BRAF inhibitor where appropriate.

In some embodiments, a PD-1 antibody agent is as disclosed in International Patent Application Publication WO2014/179664, the entirety of which is incorporated herein.

Exemplary PD-1 agents are described in Table 3.

In embodiments, a PD-1 agent is any of PD-1 agent nos. 1-94 of Table 3.

In some embodiments, an agent that inhibits PD-1 signaling binds to human PD-1. In some embodiments, an agent that inhibits PD-1 signaling binds to human PD-L1.

Exemplary PD-L1 agents are described in Table 4.

In embodiments, a PD-L1 agent is any of PD-L1 agent nos. 1-89 of Table 4.

TABLE 3
PD-1 Agents

| PD-1 Agent No. | Drug Name and Synonyms |
|---|---|
| 1 | pembrolizumab |
| | Keytruda |
| | lambrolizumab |
| | MK 3475 |
| | MK-3475 |
| | MK3475 |
| | SCH 900475 |
| | SCH-900475 |
| | SCH900475 |
| 2 | nivolumab |
| | anti-PD-1 MAb, Medarex |
| | anti-PD-1 MAb, Ono |
| | BMS 936558 |
| | BMS-936558 |
| | MDX-1106 |
| | NSC 748726 |
| | NSC-748726 |
| | ONO 4538 |
| | ONO-4538 |
| | Opdivo |
| 3 | Tripolibamab |
| | anti PD-1 monoclonal antibody, |
| | Shanghai Junshi Biosciences |
| | JS 001 |
| | JS-001 |
| | JS001 |
| | TAB 001 |
| | TAB-001 |
| | TAB001 |
| 4 | sintilimab |
| | anti-PD1 MAb, Innovent Biologies |
| | IBI-308 |
| | IBI308 |
| 5 | cemiplimab |
| | REGN 2810 |
| | REGN-2810 |
| | REGN2810 |
| | SAR-439684 |
| | SAR439684 |
| 6 | spartalizumab |
| | anti-PD-1 antibody, CoStim |
| | anti-PD-1 antibody, Novartis |
| | cancer immunotherapies, CoStim |
| | PDR-001 |
| | PDR001 |
| 7 | camrelizumab |
| | HR-301210 |
| | HR301210 |
| | INCSHR-1210 |
| | INCSHR1210 |
| | SHR-1210 |
| | SHR1210 |
| 8 | BGB-A317 |
| | BGB A317 |
| | BGBA317 |
| | PD-1 MAb, BeiGene |
| | Tislelizumab |
| 9 | BCD-100, Biocad |
| | anti-PD1 monoclonal antibody, Biocad |
| | BCD100, Biocad |
| 10 | MGA-012 |
| | INCMGA 00012 |
| | INCMGA 0012 |
| | INCMGA-00012 |
| | INCMGA-0012 |
| | INCMGA00012 |
| | INCMGA0012 |
| | MGA 012 |
| | MGA012 |
| 11 | MEDI-0680 |
| | AMP-514 |
| | AMP514 |
| | MEDI0680 |
| 12 | JNJ-63723283 |
| | JNJ-3283 |
| | JNJ3283 |
| | JNJ63723283 |
| 13 | genolimzumab |
| | CBT-501 |
| | CBT501 |
| | GB-226 |
| | GB226 |
| | recombinant PD-1 humanized |
| | monoclonal antibody, Genor Biopharma |
| 14 | AGEN-2034 |
| | AGEN 2034 |
| | AGEN2034 |
| | PD-1 antagonist, Agenus |
| 15 | XmAb-20717 |
| | PD-1/CTLA-4 bispecific antibody, |
| | Xencor |
| | XmAb20717 |
| 16 | dostarlimab (TSR-042) |
| 17 | Sym-021 |
| | anti-PD-1, Symphogen |
| | Sym021 |
| 18 | PF-06801591 |
| | anti-PD-1 antibody, Pfizer |
| | PF06801591 |
| 19 | MGD-013 |
| | MGD013 |
| 20 | LZM-009 |
| | LZM009 |
| 21 | iPD1 CD19 eCAR T cells, Marino |
| | Biotechnology |
| | autologous Anti-CD19 4-1BB CAR T |
| | Cells, Marino Biotechnology |
| 22 | HX-008 |
| | HX008 |
| | recombinant humanized anti-PD-1 |
| | monoclonal antibody |
| 23 | HLX-10 |
| | anti-PD-1 monoclonal antibody, Henlius |
| | HLX10 |
| 24 | GLS-010 |
| | AB 122 |
| | AB-122 |
| | AB122 |
| | GLS010 |
| | GLS010 |
| 25 | Cytoplasmic activated PD-1 CAR19 T |
| | cell therapy, Pregene |
| | CAR19 T cells carrying cytoplasmic |
| | activated PD1 therapy, Pregene |
| 26 | CS-1003 |
| | CS1003 |
| 27 | BI-754091 |
| | anti- PD-1 monoclonal antibody, |
| | Boehringer Ingelheim |
| | BI 754091 |
| 28 | AK-105, Akeso Biopharma |
| | AK105, Akeso Biopharma |
| 29 | AK-104 |
| | AK104 |
| | PDCD1 antibody, Akeso |
| 30 | AK-103 |
| | AK103 |
| 31 | ABBV-181 |
| | ABBV 181 |
| | ABBV181 |
| 32 | XmAb-23104 |
| | CD278/PDCD1 antibody, Xencor |
| | XmAb 23104 |
| | XmAb23104 |
| 33 | SNA-01 |
| | SNA 01 |
| | SNA01 |
| 34 | sd-rxRNA anticancer therapy, RXi |
| | Pharmaceuticals |
| | SD-RXRNA, RXi Pharmaceuticals |
| 35 | RXI-762 |
| | cancer immunotherapies, MirImmune |
| | cancer immunotherapies, RXi |
| | Pharmaceuticals |
| 36 | RXI762 |
| | RB-M1 |
| | anti-mesothelin CAR-T therapy, Refuge |
| | Biotech |
| | RB M1 |
| | RBM1 |
| 37 | RB-H21 |
| | anti-HER2 CAR-T therapy, Refuge |
| | Biotech |
| | RB H21 |
| | RBH21 |
| 38 | RB-1916 |
| | RB 1916 |
| | RB1916 |
| 39 | PRS-332 |
| | PDCD1 antibody, Pieris |
| | PRS 332 |
| | PRS332 |
| 40 | PEG-interferon-alpha2b + PD-1 |
| | inhibitor, PharmaEssentia |
| | P-1101 + PD-1 inhibitor, PharmaEssentia |
| | P1101 + PD-1 inhibitor, PharmaEssentia |
| 41 | PD-1/PD-L1 immune checkpoint |
| | inhibitors, ImmunoBrain Checkpoint |
| | PD-1/PD-L1 immune checkpoint |
| | inhibitors, Lundbeck |
| 42 | PD-1 knockout CAR, Cellular |
| | Biomedicine Group |
| | PD-1 knockout chimeric antigen |
| | receptor, Cellular Biomedicine Group |
| 43 | PD-1 inhibitors, Globavir |
| 44 | mimotope vaccine, Imugene |
| 45 | MGD-019 |
| | MGD019 |
| | PD-1/CTLA4, MacroGenics |
| | MCLA-134 |
| 46 | MCLA134 |
| | PDCD1/HAVCR1 antibody, Merus |
| 47 | MAX-10129 |
| | MAX 10129 |
| | MAX10129 |
| 48 | KF-082 |
| | KF 082 |
| | KF082 |
| | PD1 antibody, Akeso |
| 49 | JTX-4014 |
| | JTX4014 |
| 50 | JBI-426 |
| | JBI 426 |
| | JBI426 |
| 51 | immuno-oncology therapy, Olipass |
| 52 | IKT-202 |
| | IKT202 |
| 53 | ENUM-388D4 |
| | anti-PD-1 antibodies, Enumeral |
| | Biomedical |
| | ENUM 388D4 |
| 54 | DARPin anticancer therapy, Molecular |
| | Partners |
| | PD-1/VEGF multi-DARPin cancer |
| | therapy, Molecular Partners |
| 55 | CX-188 |
| | CX188 |
| | PD-1 probodies, CytomX Therapeutics |
| 56 | CS-4100 |
| | CS 4100 |
| | CS4100 |
| 57 | CAB-PD-1 |
| | CAB-PD 1 |
| | CABPD1 |
| 58 | BH-2950 |
| | BH2950 |
| | PD-1/ERRB2 antibody, Hanmi |
| 59 | BH-2941 |
| | anti PD-1/PD-L1 BsAb, Hanmi |
| | BH2941 |
| | PD-1/PD-L1 antibody, Hanmi |
| 60 | AT-16201 |
| | AT 16201 |
| | AT16201 |
| 61 | anticancer therapeutics, Bristol-Myers |
| | Squibb |
| | anticancer therapeutics, Halozyme |
| | nivolumab, Bristol-Myers Squibb |
| | nivolumab, Halozyme |
| 62 | anti-PD1 MAbs, Aduro BioTech |
| 63 | anti-PD-1 scFv + anti-CTLA4 scFv, |
| | Anaeropharma |
| | anti-CTLA4 scFv + anti-PD-1 scFv, |
| | Anaeropharma |
| 64 | anti-PD-1 antibody, Tikcro |
| 65 | anti PD-1/TAA-3 antibody, Hanmi |
| | anti TAA-3/PD-1 antibody, Hanmi |
| | PD1/TAA3 antibody, Hanmi |
| 66 | anti PD-1/TAA-2 antibody, Hanmi |
| | anti TAA-2/PD-1 antibody, Hanmi |
| | PD1/TAA2 antibody, Hanmi |
| 67 | anti PD-1/TAA-1 antibody, Hanmi |
| | anti PD-1/TAA-1 antibody, Innovent |
| | immuno-oncology programme, Hanmi |
| 68 | AM-0001 |
| | AM 0001 |
| | anti-PDl antibody, ARMO BioSciences |
| 69 | AK-123 |
| | AK 123 |
| | AK123 |
| | PD1 bispecific antibody, Akeso |
| 70 | AK-112 |
| | AK112 |
| | PD-1 bispecific antibody, Akeso |
| 71 | ENUM-244C8 |
| | anticancer MAbs, Enumeral Biomedical |
| | ENUM 244C8 |
| 72 | Sym-016 |
| | anticancer therapies, Symphogen-2 |
| | Sym016 |
| 73 | STI-A1110 |
| | STI-1110 |
| | STI-A110X |
| | STI1110 |
| | STIA110X |
| | STIA1110 |
| 74 | PEG-MP7 |
| | PEGMP7 |
| 75 | PD-1 peptide antagonists, Aurigene |
| | Discovery Technologies |
| 76 | mAb B60-55 |
| | B60 55 |
| | B60-55 |
| | mAb B60 55 |
| 77 | INDUS-903 |
| | INDUS903 |
| 78 | immune checkpoint inhibitors, |
| | TheraVectys |
| 79 | IMM-1802 |
| | IMM 1802 |
| | IMM1802 |
| 80 | cancer immunotherapies, Immunotherapy |
| | Nantibody |
| 81 | bispecific PD-1/OX40 antibody, Immune |
| | Pharmaceuticals |
| | TNFRSF4/PD1 antibody, Immune |
| | Pharmaceuticals |
| 82 | BH-2922 |
| | BH 2922 |
| | BH2922 |
| | EGFR/PD-1 antibody, Hanmi |
| 83 | AUNP-12 |
| | AUNP 12 |
| | AUNP12 |
| | AUR 012 |
| | AUR-012 |
| | W-014A |
| | W014A |
| 84 | AP-106 |
| | AP106 |
| 85 | anticancers, ArQule |
| | anticancers, Beryllium |
| 86 | anti-PD1 antibody, Premier Biomedical |
| 87 | anti-PD-1 MAbs, PxRadia |
| | anticancer immunotherapy, PxRadia |
| 88 | anti-PD-1 MAbs, Kadmon |
| | Pharmaceuticals |
| 89 | anti-PD-1 MAb, Immunovo |
| 90 | anti-PD-1 bispecific antibodies, Innovent |
| | Biologics |
| | anti-PD-1 bispecific antibodies, Eli Lilly |
| | bispecific molecules, Eli Lilly |
| | bispecific molecules, Innovent Biologics |
| | PD-1 bispecific antibody, Innovent |
| | Biologics |
| 91 | anti PD-1 MAbs, Mabquest |
| | anti PD-1 MAbs, Cellectis |
| 92 | ANB-011 |
| | ANA-011 |
| | ANA011 |
| | ANB011 |
| 93 | AN-2005 |
| | AN2005 |
| 94 | AMP-224 |
| | AMP224 |
| | GSK 2661380 |
| | GSK-2661380 |
| | GSK2661380 |

In some embodiments, the PD-1 signaling agent is a PD-L1 inhibitor provided in Table 4.

TABLE 4
PD-L1 inhibitors

| PD-L1 Agent No. | Drug Name and Synonyms |
|---|---|
| 1 | durvalumab |
| | Imfinzi |
| | MEDI 4736 |
| | MEDI-4736 |
| | MEDI4736 |
| 2 | avelumab |
| | anti PD-L1, Merck KGaA |
| | Bavencio |
| | MSB-0010718C |
| | MSB0010718C |
| | PF 06834635 |
| | PF-06834635 |
| 3 | atezolizumab |
| | MPDL-3280A |
| | MPDL3280A |
| | RG 7446 |
| | RG-7446 |
| | RG7446 |
| | RG7746 |
| | RO-5541267 |
| | RO5541267 |
| | Tecentriq |
| 4 | WBP- 3155 |
| | CS 1001 |
| | CS-1001 |
| | CS1001 |
| | WBP 3155 |
| 5 | IO-103 |
| | IO 103 |
| | IO103 |
| 6 | CX-072 |
| | CX072 |
| | PD-L1 probodies, CytomX Therapeutics |
| 7 | CA-170 |
| | AUPM-170 |
| | AUPM170 |
| | PD-L1 inhibitors, Aurigene Discovery |
| | Technologies |
| | PD-L1 inhibitors, Curis |
| 8 | BGB-A333 |
| | anti PD-L1 MAb, BeiGene |
| | BGB A333 |
| | BGBA333 |
| 9 | SHR-1316 |
| | HTI-1088 |
| | HTI1088 |
| | SHR1316 |
| 10 | MSB-2311 |
| | MSB 2311 |
| | MSB2311 |
| 11 | M-7824 |
| | M7824 |
| | MSB0011359C |
| 12 | LY-3300054 |
| | anti PD-L1 Mab, Eli Lilly |
| | LY 3300054 |
| | LY3300054 |
| 13 | KN-035 |
| | 3D-2-02-0015 |
| | KN035 |
| 14 | FS-118 |
| | FS118 |
| | LAG3/CD274 antibody, f-star |
| 15 | FAZ-053 |
| | anti-PD-L1 antibody, Novartis-2 |
| | FAZ053 |
| 16 | CK-301 |
| | anti-PD-L1 antibody, Checkpoint |
| | Therapeutics |
| | anti-PD-L1 antibody, TG Therapeutics |
| | CK301 |
| 17 | BMS-936559 |
| | anti-PD-L1 antibody, Bristol-Myers |
| | Squibb |
| | BMS936559 |
| | MDX-1105 |
| | MDX1105 |
| 18 | BCD-135 |
| | BCD135 |
| 19 | AK-106, Akeso |
| | AK106, Akeso |
| 20 | trastuzumab/PD-L1 fusion protein, |
| | Avacta |
| 21 | STI-A1014 |
| | STI-1014 |
| | STI1014 |
| | STIA1014 |
| | ZKAB-001 |
| | ZKAB001 |
| 22 | SNA-02 |
| | anti-PD-L1 aptamer, Fountain BioPharma |
| | SNA 02 |
| | SNA02 |
| 23 | sd-rxRNA anticancer therapy, RXi |
| | Pharmaceuticals |
| | SD-RXRNA, RXi Pharmaceuticals |
| 24 | rituximab/PD-L1 fusion protein, Avacta |
| 25 | PD-L1/LAG-3 bispecific programme, |
| | Avacta |
| 26 | PD-L1 inhibitors, Hitgen |
| | PDL1 inhibitors, Hitgen |
| 27 | PD-L1 gene therapy, enGene |
| 28 | PD-L1 CAR.TNK, Sorrento Therapeutics |
| | PDL1.taNK, NantKwest |
| | PDL1.taNK, Sorrento Therapeutics |
| 29 | PD-L1 antagonist, Arbutus Biopharma |
| | checkpoint inhibitors, Arbutus Biopharma |
| 30 | PD-1/PD-L1 immune checkpoint |
| | inhibitors, ImmunoBrain Checkpoint |
| | PD-1/PD-L1 immune checkpoint |
| | inhibitors, Lundbeck |
| 31 | OT-2 |
| | OT 2 |
| | OT2 |
| 32 | ND-021 |
| | ND 021 |
| | ND021 |
| | PD-L1 antibody, Numab |
| 33 | MT-5050 |
| | MT 5050 |
| | MT5050 |
| 34 | MEDI-1109 |
| | MEDI 1109 |
| | MEDI1109 |
| | TNFRSF4/PD-L1 antibody, AstraZeneca |
| 35 | MCLA-145 |
| | CD274 antibody, Merus |
| | MCLA145 |
| 36 | MAX-10129 |
| | MAX 10129 |
| | MAX10129 |
| 37 | KY-1043 |
| | KY1043 |
| 38 | KY-1003 |
| | KY1003 |
| 39 | KD-033 |
| | KD033 |
| 40 | JS-003 |
| | CD274 antibody, Shanghai |
| | JS003 |
| 41 | JBI-426 |
| | JBI 426 |
| | JBI426 |
| 42 | ipilimumab/PD-L1, Avacta |
| | ipilimumab/PD-L1 fusion protein, Avacta |
| 43 | immuno-oncology therapy, Olipass |
| 44 | immuno-oncology programme, Avacta |
| 45 | IMM-25 |
| | IMM 25 |
| | IMM25 |
| 46 | IMC-2102 |
| | CD274 antibody, ImmuneOncia-2 |
| | IMC 2102 |
| 47 | IMC-2101 |
| | CD274/U1 bispecific antibody, |
| | ImmuneOncia |
| | IMC 2101 |
| 48 | IMC-001 |
| | IMC 001 |
| | IMC001 |
| | STI-A 1015 |
| | STI-A-1015 |
| | STI-A1015 |
| | STIA1015 |
| 49 | IKT-703 |
| | IKT 703 |
| | IKT703 |
| 50 | IKT-201 |
| | IKT201 |
| 51 | HLX-20 |
| | HLX20 |
| 52 | GX-P2 |
| | GXP2 |
| 53 | FPT-155 |
| | CD80-Fc, Five Prime |
| | FPT155 |
| 54 | CS-4100 |
| | CS 4100 |
| | CS4100 |
| 55 | CBT-502 |
| | CBT502 |
| | TQB-2450 |
| | TQB2450 |
| 56 | cancer therapy, VLP Therapeutics |
| 57 | BH-2941 |
| | anti-PD-1/PD-L1 BsAb, Hanmi |
| | BH2941 |
| | PD-1/PD-L1 antibody, Hanmi |
| 58 | bevacizumab/PD-L1, Avacta |
| | bevacizumab/PD-L1 fusion protein, |
| | Avacta |
| 59 | AVA-004 |
| | anti PD-L1 affimers, Avacta |
| | AVA004 |
| | PDL1-182 Fc, Avacta |
| 60 | AUPM-327 |
| | AUPM327 |
| | CA-327 |
| | CA327 |
| | PD-L1/TIM3 dual inhibitors, Aurigene |
| | PD-L1/TIM3 dual inhibitors, Curis |
| | TIM3/PD-L1 dual inhibitors, Aurigene |
| | TIM3/PD-L1 dual inhibitors, Curis |
| 61 | AP-105, AP Biosciences |
| | AP105, AP Biosciences |
| 62 | anti-PD-L1 RRV, Tacogen |
| | cancer-selective RRVs, Tocagen |
| 63 | anti-PD-L1 antibody, Tikcro |
| 64 | ALPN-202 |
| | ALPN202 |
| 65 | ALN-PDL |
| | PD-L1 siRNA, Alnylam |
| 66 | triple modulator, Fate Therapeutics |
| 67 | Toca-531 |
| | Toca 531 |
| 68 | STI-B010X |
| | STIB010X |
| 69 | STI-A1010 |
| | STI-A100X |
| | STI-A1011 |
| | STI-A1012 |
| | STIA100X |
| | STIA1010 |
| | STIA1011 |
| | STIA1012 |
| 70 | programmed death ligand (PD-L1) |
| | inhibitors, Regeneron |
| | programmed death ligand (PD-L1) |
| | inhibitors, Sanofi |
| 71 | PD-L1 × CTLA-4 immune checkpoints |
| | antibodies, IGM Biosciences |
| 72 | PD-L1 MAb, Kadmon Corporation |
| | PD-L1 MAb, Nantong Jinghua |
| | Pharmaceuticals |
| 73 | PD-L1 MAb, Abeome |
| 74 | PD-L1 inhibitor, Bristol-Myers Squibb |
| 75 | PD-L1 immune checkpoint antibodies, |
| | IGM Biosciences |
| 76 | mAb B60-55 |
| | B60 55 |
| | B60-55 |
| | mAb B60 55 |
| 77 | immune checkpoint inhibitors, |
| | TheraVectys |
| 78 | IMM-2504 |
| | IMM 2504 |
| | IMM2504 |
| 79 | IMM-2503 |
| | IMM 2503 |
| | IMM2503 |
| 80 | IMM-2502 |
| | IMM 2502 |
| | IMM2502 |
| 81 | HTI-1316 |
| | HTI1316 |
| 82 | cancer immunotherapies, Immunotherapy |
| | Nantibody |
| 83 | bispecific PD-L1/BCMA antibody, |
| | Immune Pharmaceuticals |
| 84 | BIPI |
| | atezolizumab/IL-2 immunocytomkine, |
| | Shanghai Zhangjiang Biotech |
| 85 | BGB-108 |
| | BGB108 |
| | PD-L1 MAb |
| | PDL1 MAb |
| 86 | BBI-801 |
| | BBI801 |
| 87 | anticancers, ArQule |
| | anticancers, Beryllium |
| 88 | anti-PD-L1 antibody, CoStim |
| | anti-PD-L1 antibody, Novartis |
| 89 | anti-c-Met/PD-L1 bispecific antibody, |
| | Sorrento Therapeutics |
| | MET/PD-L1 antibody, Sorrento |

In some embodiments, a PD-1-binding agent is glycosylated and one or more sites. As used herein, “glycan” is a sugar polymer (moiety) component of a glycoprotein. The term “glycan” encompasses free glycans, including glycans that have been cleaved or otherwise released from a glycoprotein. In some embodiments, present disclosure provides a composition comprising one or more glycoforms of a heavy chain, light chain, and/or antibody agent as described herein. In some embodiments, a glycan is N-linked to an Fc region. In some embodiments, a PD-1-binding agent is glycosylated at Asn297 (Kabat numbering).

The term “glycoform” is used herein to refer to a particular form of a glycoprotein. That is, when a glycoprotein includes a particular polypeptide that has the potential to be linked to different glycans or sets of glycans, then each different version of the glycoprotein (i.e., where the polypeptide is linked to a particular glycan or set of glycans) is referred to as a “glycoform.” In some embodiments, a provided composition comprises a plurality of glycoforms of one or more of an heavy chain, light chain, and/or antibody agent as described herein.

In some embodiments a PD-1-binding agent binds with high affinity to human and cynomolgus monkey PD-1. In some embodiments, binding of a PD-1-binding agent can be characterized by surface plasma resonance (SPR). In some embodiments, SPR measurements may demonstrate or confirm binding of a PD-1 signaling agent a to human and/or a cynomolgus monkey PD-1 Fc fusion. In some embodiments, a PD-1-binding agent binds human and cynomolgus PD-1 with a fast association rate, slow dissociation rate, and high affinity.

In some embodiments, antagonist activity of a PD-1-binding agent in blocking the PD-1/PD-L1 or PD-L2 interaction may be confirmed or determined using a flow cytometry-based assay that measured binding of labeled PD-L1 and PD-L2 expressed as a mouse IgG1 Fc fusion proteins (PD-L1 mFc or PD-L2 mFc) to PD-1-expressing cells. In some embodiments, a PD-1-binding agent can efficiently block PD-1/PD-L1 and PD-1/PD-L2 binding compared to an IgG4 isotype control.

In some embodiments, a PD-1-binding agent can effectively neutralize PD-1 activity (e.g., can inhibit binding of PD-1 to PD-L1 and PD-L2). In some embodiments, functional antagonist activity of a PD-1-binding agent may be confirmed or determined in a mixed lymphocyte reaction (MLR) demonstrating enhanced interleukin (IL)-2 production upon addition of a PD-1-binding agent. In some embodiments, a MLR assay may be carried out using primary human CD4+ T cells as responders and human dendritic cells as stimulators.

In some embodiments, a PD-1 signaling agent is expressed from a vector comprising one or more nucleic acid sequences encoding a PD-1-binding immunoglobulin heavy chain variable domain polypeptide and/or a PD-1-binding immunoglobulin light chain variable domain polypeptide. In some embodiments, a PD-1 signaling agent is expressed from a vector comprising one or more nucleic acid sequences encoding a PD-1-binding immunoglobulin heavy chain polypeptide and/or a PD-1-binding immunoglobulin light chain polypeptide. The vector can be, for example, a plasmid, episome, cosmid, viral vector (e.g., retroviral or adenoviral), or phage. Suitable vectors and methods of vector preparation are well known in the art (see, e.g., Sambrook et al., Molecular Cloning, a Laboratory Manual, 3rd edition, Cold Spring Harbor Press, Cold Spring Harbor, N.Y. (2001), and Ausubel et al, Current Protocols in Molecular Biology, Greene Publishing Associates and John Wiley & Sons, New York, N.Y. (1994)).

In some embodiment, vector(s) for expression of PD-1-binding agents further comprises expression control sequences, such as promoters, enhancers, polyadenylation signals, transcription terminators, internal ribosome entry sites (IRES), and the like, that provide for the expression of the coding sequence in a host cell. Exemplary expression control sequences are known in the art and described in, for example, Goeddel, Gene Expression Technology: Methods in Enzymology, Vol. 185, Academic Press, San Diego, Calif. (1990).

The vector(s) comprising the nucleic acid(s) encoding PD-1-binding agents of the present disclosure can be introduced into a host cell that is capable of expressing the polypeptides encoded thereby, including any suitable prokaryotic or eukaryotic cell. Some preferable qualities of host cells include easy and reliable growth, a reasonably last growth rate, having well-characterized expression systems, and/or ease/efficient transformation or transfection.

In some embodiments, mammalian cells are utilized. A number of suitable mammalian host cells are known in the art, and many are available from the American Type Culture Collection (ATCC, Manassas, Va.). Examples of suitable mammalian cells include, but are not limited to, Chinese hamster ovary cells (CHO) (ATCC No. CCL61). CHO DHFR-cells (Urlaub et al, Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70).

Further exemplary mammalian host cells include primate cell lines and rodent cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, mouse L-929 cells, and BHK or HaK hamster cell lines, all of which are available from the ATCC. Methods for selecting suitable mammalian host cells and methods for transformation, culture, amplification, screening, and purification of cells are known in the art.

In some embodiments, the mammalian cell is a human cell. For example, the mammalian cell can be a human lymphoid or lymphoid derived cell line, such as a cell line of pre-B lymphocyte origin. Examples of human lymphoid cells lines include, without limitation, RAMOS (CRL-1596), Daudi (CCL-213), EB-3 (CCL-85), DT40 (CRL-2111), 18-81 (Jack et al, Proc. Natl. Acad. Sci. USA, 85: 1581-1585 (1988)), Raji cells (CCL-86), and derivatives thereof.

In some embodiments, a PD-1-binding agent is formulated as a pharmaceutical composition, containing one or a combination of monoclonal antibodies, or antigen-binding portion(s) thereof, formulated with a pharmaceutically acceptable carrier. An anti-PD-1 antibody agent may be formulated alone or in combination with other drugs (e.g., as an adjuvant). For example, a PD-1-binding agent can be administered in combination with other agents for the treatment or prevention of the diseases disclosed herein (e.g., cancer).

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it may be useful to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the ease of sterile powders is the preparation of sterile injectable solutions, such methods of preparation may include vacuum drying and freeze-drying (lyophilization) to yield a powder of the active ingredient plus any additional desired ingredient from a previously sterile-filtered solution thereof.

In some embodiments, a therapeutic composition is formulated as a sterile liquid. In some embodiments, the composition is free from visible particles. In some embodiments, the composition is formulated in a buffer (e.g., a citrate buffer). In some embodiments, the composition comprises a PD-1-binding agent and two or more of the following: citrate, arginine, sodium chloride and polysorbate 80.

In some embodiments, a therapeutic composition of the present disclosure (e.g., a PD-1 binding agent) is aseptically filled into a clear glass vial. In some embodiments, such a glass vial is stoppered with a chlorobutyl elastomer stopper laminated with fluoropolymer and sealed with an aluminum overseal.

In some embodiments, a PD-1 signaling agent is stored at 2-8° C. In some embodiments, a drug product of the present disclosure is free of preservatives.

### Administration of PD-1 Signaling Agents

As described herein, provided methods comprise administering a PD-1 signaling agent to a patient, a subject, or a population of subjects according to a regimen that achieves clinical benefit.

Provided methods can provide various benefits (e.g., a clinical benefit). In embodiments, a method described herein achieves a clinical benefit. In embodiments, a clinical benefit is stable disease (SD). In embodiments, a clinical benefit is a partial response (PR). IN embodiments, a clinical benefit is a complete response (CR).

In embodiments, a combination therapy achieves a clinical benefit for each therapy administered to a patient. For example, a combination therapy may improve a clinical benefit obtained with a PD-1 inhibitor (e.g., any anti-PD-1 antibody described herein).

In embodiments, a patient or subject is an animal. In embodiments, a patient or subject is a human.

In some embodiments, the regimen comprises at least one parental dose of a PD-1 binding agent. In some embodiments, the regimen comprises a plurality of parental doses.

In some embodiments, the parental dose is an amount of a PD-1 signaling agent is within a range of about 5 to about 5000 mg (e.g., about 5 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 2000 mg, about 3000 mg, about 4000 mg, about 5000 mg, or a range defined by any two of the foregoing values). In some embodiments, the parental dose of a PD-1 signaling agent is 500 mg or 1000 mg.

In some embodiments, the dose is in an amount relative to body weight. In some embodiments, the parental dose of a PD-1 signaling agent is within a range of about 0.01 mg/kg to 100 mg/kg of animal or human body weight; however, doses below or above this exemplary range are within the scope of the invention. The daily parenteral dose can be about 0.01 mg/kg to about 50 mg/kg of total body weight (e.g., about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12 mg/kg, about 15 mg/kg, about 20 mg/kg, or a range defined by any two of the foregoing values).

In some embodiments, a composition that delivers a PD-1-binding agent (e.g., an anti-PD-1 antibody) is administered to a patient at a dose of about 1, 3 or 10 mg/kg. In some embodiments, a PD-1-binding agent (e.g., an anti-PD-1 antibody) is administered according to a regimen that delivers a dose of about 1, 3 or 10 mg/kg every two weeks. In some embodiments, a PD-1-binding agent (e.g., an anti-PD-1 antibody) is administered according to a regimen that delivers a dose of about 1, 3 or 10 mg/kg every three weeks. In some embodiments, a PD-1-binding agent (e.g., an anti-PD-1 antibody) is administered according to a regimen that delivers a dose of about 1, 3 or 10 mg/kg every four weeks. In some embodiments, a PD-1-binding agent (e.g., an anti-PD-1 antibody) is administered according to a regimen that delivers a dose of about 1 mg/kg every three weeks. In some embodiments, a PD-1-binding agent (e.g., an anti-PD-1 antibody) is administered according to a regimen that delivers a dose of about 3 mg/kg every three weeks. In some embodiments, a PD-1-binding agent (e.g., an anti-PD-1 antibody) is administered according to a regimen that delivers a dose of about 10 mg/kg every three weeks.

In some embodiments, a composition that delivers a PD-1-binding agent (e.g., an anti-PD-1 antibody) is administered to a patient at a dose of about 400 mg. In some embodiments, a PD-1-binding agent (e.g., an anti-PD-1 antibody) is administered according to a regimen that delivers a dose of about 400 mg every two weeks. In some embodiments, a PD-1-binding agent (e.g., an anti-PD-1 antibody) is administered according to a regimen that delivers a dose of about 400 mg every three weeks. In some embodiments, a PD-1-binding agent (e.g., an anti-PD-1 antibody) is administered according to a regimen that delivers a dose of about 400 mg every four weeks.

In some embodiments, a composition that delivers a PD-1-binding agent (e.g., an anti-PD-1 antibody) is administered to a patient at a dose of about 500 mg. In some embodiments, a PD-1-binding agent (e.g., an anti-PD-1 antibody) is administered according to a regimen that delivers a dose of about 500 mg every two weeks. In some embodiments, a PD-1-binding agent (e.g., an anti-PD-1 antibody) is administered according to a regimen that delivers a dose of about 500 mg every three weeks. In some embodiments, a PD-1-binding agent (e.g., an anti-PD-1 antibody) is administered according to a regimen that delivers a dose of about 500 mg every four weeks.

In some embodiments, a composition that delivers a PD-1-binding agent (e.g., an anti-PD-1 antibody) is administered to a patient at a dose of about 800 mg. In some embodiments, a PD-1-binding agent (e.g., an anti-PD-1 antibody) is administered according to a regimen that delivers a dose of about 800 mg every three weeks. In some embodiments, a PD-1-binding agent (e.g., an anti-PD-1 antibody) is administered according to a regimen that delivers a dose of about 800 mg every four weeks. In some embodiments, a PD-1-binding agent (e.g., an anti-PD-1 antibody) is administered according to a regimen that delivers a dose of about 800 mg every six weeks. In some embodiments, a PD-1-binding agent (e.g., an anti-PD-1 antibody) is administered according to a regimen that delivers a dose of about 800 mg every eight weeks.

In some embodiments, a composition that delivers a PD-1-binding agent (e.g., an anti-PD-1 antibody) is administered to a patient at a dose of about 1,000 mg. In some embodiments, a PD-1-binding agent (e.g., an anti-PD-1 antibody) is administered according to a regimen that delivers a dose of about 1,000 mg every three weeks. In some embodiments, a PD-1-binding agent (e.g., an anti-PD-1 antibody) is administered according to a regimen that delivers a dose of about 1,000 mg every four weeks. In some embodiments, a PD-1-binding agent (e.g., an anti-PD-1 antibody) is administered according to a regimen that delivers a dose of about 1,000 mg every five weeks. In some embodiments, a PD-1-binding agent (e.g., an anti-PD-1 antibody) is administered according to a regimen that delivers a dose of about 1.000 mg every six weeks. In some embodiments, a PD-1-binding agent (e.g., an anti-PD-1 antibody) is administered according to a regimen that delivers a dose of about 1,000 mg every seven weeks. In some embodiments, a PD-1-binding agent (e.g., an anti-PD-1 antibody) is administered according to a regimen that delivers a dose of about 1.000 mg every eight weeks. In some embodiments, a PD-1-binding agent (e.g., an anti-PD-1 antibody) is administered according to a regimen that delivers a dose of about 1.000 mg every nine weeks.

In some embodiments, a PD-1-binding agent (e.g., an anti-PD-1 antibody) is administered according to a regimen that delivers a dose of about 500 mg every three weeks. In some embodiments, a PD-1-binding agent (e.g., an anti-PD-1 antibody) is administered according to a regimen that delivers a dose of about 1000 mg every six weeks.

In some embodiments, a PD-1-binding agent (e.g., an anti-PD-1 antibody) is administered according to a regimen that delivers a first dose of PD-1-binding agent for the first 2-6 dosing cycles (e.g., the first 3, 4, or 5 dosing cycles), and then delivers a second dose of a PD-1-binding agent for the subsequent dosing cycles until therapy is discontinued (e.g., due to disease progression or an adverse effect or as directed by a physician). In some embodiments, the duration of the first set of 2-6 dosing cycles (e.g., the first 3, 4, or S dosing cycles) is different from the duration of the subsequent dosing cycles. In embodiments, a PD-1-binding agent (e.g., an anti-PD-1 antibody) is administered according to a regimen that delivers a first dose of PD-1-binding agent once every three weeks for the first three dosing cycles, and then delivers a second dose of a PD-1-binding agent once every six weeks or more for the remaining dosing cycles (e.g., a second dose of a PD-1-binding agent once every six weeks for the remaining dosing cycles). In embodiments, a PD-1-binding agent (e.g., an anti-PD-1 antibody) is administered according to a regimen that delivers a first dose of PD-1-binding agent once every three weeks for the first four dosing cycles, and then delivers a second dose of a PD-1-binding agent once every six weeks or more for the remaining dosing cycles (e.g., a second dose of a PD-1-binding agent once every six weeks for the remaining dosing cycles). In embodiments, a PD-1-binding agent (e.g., an anti-PD-1 antibody) is administered according to a regimen that delivers a first dose of PD-1-binding agent once every three weeks for the first five dosing cycles, and then delivers a second dose of a PD-1-binding agent once every six weeks or for the remaining dosing cycles (e.g., a second dose of a PD-1-binding agent once every six weeks for the remaining dosing cycles). In some embodiments, a PD-1-binding agent (e.g., an anti-PD-1 antibody) is administered according to a regimen that delivers a first dose of PD-1-binding agent once every three weeks for the first 2-6 dosing cycles (e.g., the first 3,4, or 5 dosing cycles), and then delivers a second dose of a PD-1-binding agent once every six weeks or until therapy is discontinued (e.g., due to disease progression or an adverse effect or as directed by a physician). In some embodiments, a PD-1-binding agent (e.g., an anti-PD-1 antibody) is administered according to a regimen that delivers a first dose of a PD-1-binding agent once every three weeks for the first 3, 4, or 5 dosing cycles (e.g., the first 4 dosing cycles), and then delivers a second dose of a PD-1-binding agent once every six weeks or more until therapy is discontinued (e.g., due to disease progression or an adverse effect or as directed by a physician). In embodiments, the method comprises delivering a second dose of PD-1 signaling agent once every six weeks until therapy is discontinued.

In some embodiments the first and/or second dose of a PD-1-binding agent (e.g., an anti-PD-1 antibody) is about 100 mg to about 2,000 mg (e.g., about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg). In some embodiments the first dose and the second dose are the same. In some embodiments, the first dose and the second dose are different. In embodiments, the first dose is about 500 mg of a PD-1-binding agent (e.g., an anti-PD-1 antibody). In embodiments, the first dose is about 1000 mg of a PD-1-binding agent (e.g., an anti-PD-1 antibody).

In some embodiments, a PD-1-binding agent (e.g., an anti-PD-1 antibody) is administered according to a regimen that comprises administering an about 500 mg dose every 3 weeks for four doses followed by administering at least one about 1,000 mg dose every six weeks after the fourth dose of about 500 mg. In some embodiments, additional about 1.000 mg doses are administered every six weeks after the first about 1000 mg dose until no further clinical benefit is achieved. In some particular embodiments, a PD-1 signaling agent (e.g., an anti-PD1 antibody) is administered according to a dosing regimen that includes 500 mg for 4 cycles Q3W followed by 1000 mg Q6W.

In some embodiments, a PD-1-binding agent (e.g., an anti-PD-1 antibody) is administered according to a regimen that comprises administering a 400 mg dose every 3 weeks for four doses followed by administering at least one 800 mg dose every six weeks after the fourth 400 mg dose. In some embodiments, additional 800 mg doses are administered every six weeks after the first 800 mg dose until no further clinical benefit is achieved. In some particular embodiments, a PD-1 signaling agent (e.g., an anti-PD1 antibody) is administered according to a dosing regimen that includes 400 mg for 4 cycles Q3W followed by 800 mg Q6W.

Therapeutic or prophylactic efficacy can be monitored by periodic assessment of treated patients. For repeated administrations over several days or longer, depending on the condition, the treatment can be repeated until a desired suppression of disease symptoms occurs. However, other dosage regimens may be useful and are within the scope of the invention.

The desired dosage can be delivered by a single bolus administration of the composition, by multiple bolus administrations of the composition, or by continuous infusion administration of the composition.

In some embodiments, a PD-1 signaling agent is administered to a patient or population of subjects who has exhibited response to prior therapy. In some embodiments, the patient or population of subjects has exhibited response to a prior cancer therapy.

In some embodiments, a PD-1 signaling agent is administered to a patient or population of subjects who has not exhibited response to prior therapy. In some embodiments, the patient or population of subjects has not received or exhibited response to a prior cancer therapy.

In embodiments, a subject is resistant to treatment with an agent that inhibits PD-1. In embodiments, a subject is refractory to treatment with an agent that inhibits PD-1. In embodiments, a method described herein sensitizes the subject to treatment with an agent that inhibits PD-1.

### Combination Therapy

Provided herein are methods that comprise administering a first therapeutic agent (e.g., an immune checkpoint inhibitor) in combination with one or more additional therapeutic agents.

In embodiments, an anti-PD-1 therapy as described herein is administered in combination with one or more additional therapies (e.g., therapies as described herein). That is, a subject is treated with an anti-PD-1 therapy and one or more additional therapies is administered to a subject such that the subject receives each therapy.

In embodiments, an additional therapy is surgery. In embodiments, an additional therapy is radiotherapy. In embodiments, an additional therapy is chemotherapy. In embodiments, an additional therapy is immunotherapy.

In some embodiments, a PD-1 signaling agent is administered simultaneously or sequentially with an additional therapeutic agent, such as, for example, another antibody agent (e.g., an antibody agent that binds a checkpoint inhibitor and/or a chemotherapeutic agent). In some embodiments, a PD-1 signaling agent is administered before, during, or after administration of an additional therapeutic agent. In some embodiments, a PD-1 signaling agent is administered before, during, or after administration of a chemotherapeutic agent.

An anti-PD-1 antibody agent may be administered alone or in combination with other drugs (e.g., as an adjuvant). For example, the PD-1 binding agent can be administered in combination with other agents for the treatment or prevention of the diseases disclosed herein (e.g., cancer). In this respect, the PD-1 binding agent can be used in combination with at least one other anticancer agent including, for example, any chemotherapeutic agent known in the art, ionization radiation, small molecule anticancer agents, cancer vaccines, biological therapies (e.g., other monoclonal antibodies, cancer-killing viruses, gene therapy, and adoptive T-cell transfer), and/or surgery.

Administration of a PD-1 signaling agent simultaneously or sequentially with an additional therapeutic agent is referred to herein as “combination therapy.” In combination therapy, a PD-1 signaling agent can be administered prior to (e.g., 5 minutes, 15 minutes, 30 minutes, 45 minutes, 1 hour, 2 hours, 4 hours, 6 hours, 12 hours, 24 hours, 48, hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the additional therapeutic agent to a subject in need thereof. In some embodiments a PD-1 signaling agent and an additional therapeutic agent are administered 1 minute apart, 10 minutes apart, 30 minutes apart, less than 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart, or no more than 48 hours apart.

Checkpoint Inhibitors

In embodiments, an additional therapy is an immunotherapy. In embodiments, an immunotherapy comprises administration of one or more further immune checkpoint inhibitors (e.g., administration of one, two, three, four, or more further immune checkpoint inhibitors).

Exemplary immune checkpoint targets for inhibition include: PD-1 (e.g., inhibition via anti-PD-1, anti-PD-L1, or anti-PD-L2 therapies), CTLA-4, TIM-3, TIGIT, LAGs (e.g., LAG-3), CEACAM (e.g., CEACAM-1, -3 and/or -5), VISTA, BTLA, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, TGFR (e.g., TGFR beta), B7-H1, B7-H4 (VTCN1), OX-40, CD137, CD40, IDO, and CSF-1R. Accordingly, agents that inhibit of any of these molecules can be used in combination with an anti-PD-1 therapy described herein.

In embodiments, an immune checkpoint inhibitor is a CTLA-4 inhibitor (e.g., an antibody, an antibody conjugate, or an antigen-binding fragment thereof). In embodiments, a CTLA-4 inhibitor is a small molecule, a nucleic acid, a polypeptide (e.g., an antibody), a carbohydrate, a lipid, a metal, or a toxin. In embodiments, a CTLA-4 inhibitor is a small molecule. In embodiments, a CTLA-4 inhibitor is a CTLA-4 binding agent. In embodiments, a CTLA-4 inhibitor is an antibody, an antibody conjugate, or an antigen-binding fragment thereof.

In embodiments, a CTLA-4 inhibitor is a CTLA-4 antibody described herein. In embodiments, a CTLA-4 inhibitor is ipilimumab (Yervoy), AGEN1884, or tremelimumab.

In embodiments, a checkpoint inhibitor is a small molecule, a nucleic acid, a polypeptide (e.g., an antibody), a carbohydrate, a lipid, a metal, a toxin, or a binding agent. In embodiments, a checkpoint inhibitor is an antibody, an antibody conjugate, or an antigen-binding fragment thereof.

In embodiments, an immune checkpoint inhibitor is an agent that inhibits TIM-3, CTLA-4, LAG-3, TIGIT, IDO or CSF1R.

For female patients of childbearing potential, it is preferable that the patient have a negative serum pregnancy test within 72 hours prior to the date of administration of the first dose of an anti-PD-1 binding agent. It is also preferable that female patients of childbearing potential and male patients agree to use 2 adequate methods of contraception with their partner. In some embodiments, a patient agrees to use 2 methods of contraception starting with the screening visit through 150 days after the last dose of study therapy.

### V. Compositions

In some aspects, also provided herein are compositions (e.g., pharmaceutical composition) comprising any of the anti-CTLA4 binding proteins described herein. In some aspects, also provided herein are compositions (e.g., pharmaceutical composition) comprising any of the PD-1 signaling agents described herein. In some embodiments, the anti-CTLA4 binding proteins described herein and the PD-1 signaling agents described herein are formulated in the same composition.

Therapeutic formulations are prepared for storage by mixing the active ingredient having the desired degree of purity with optional pharmaceutically acceptable carriers, excipients or stabilizers (Remington: The Science and Practice of Pharmacy, 20th Ed., Lippincott Williams & Wilkins, Pub., Gennaro Ed., Philadelphia, Pa. 2000). Acceptable carriers, excipients, or stabilizers are nontoxic to recipients at the dosages and concentrations employed, and include buffers antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers can be used to control the pH in a range which optimizes the therapeutic effectiveness, especially if stability is pH dependent. Buffers can be present at concentrations ranging from about 20 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may be comprised of histidine and trimethylamine salts such as Tris.

Preservatives can be added to prevent microbial growth, and are typically present in a range from about 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Tonicity agents can be present in any amount between about 0.1% to about 25% by weight or between about 1 to about 5% by weight, taking into account the relative amounts of the other ingredients. In some embodiments, tonicity agents include polyhydric sugar alcohols, trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, omithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin ordextran.

Non-ionic surfactants or detergents (also known as “wetting agents”) can be present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/mi or about 0.07 mg/ml to about 0.2 mg/ml. In some embodiments, non-ionic surfactants are present in a range of about 0.001% to about 0.1% w/v or about 0.01% to about 0.1% w/v or about 0.01% to about 0.025% w/v.

Suitable non-ionic surfactants include polysorbates (20, 40, 60, 65, 80, etc.), poloxamers (184, 188, etc.), PLURONIC® polyols, TRITON®, polyoxyethylene sorbitan monoethers (TWEEN®-20, TWEEN-80, etc.), lauromacrogol 400, polyoxyl 40 stearate, polyoxyethylene hydrogenated castor oil 10, 30 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the formulations to be used for in vivo administration, they must be sterile. The formulation may be rendered sterile by filtration through sterile filtration membranes. The therapeutic compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

An anti-CTLA4 binding protein described herein (e.g., anti-CTLA4 antibody or antigen-binding fragment thereof) can be used alone or in combination with other therapeutic agents such is in the methods described herein. The term “in combination with” encompasses two or more therapeutic agents (e.g., an anti-CTLA4 binding protein and a therapeutic agent) that are included in the same or separate formulations. In some embodiments, “in combination with” refers to “simultaneous” administration, in which case administration of the anti-CTLA4 binding protein of the invention occurs simultaneously to the administration of the one or more additional therapeutic agents (e.g., at the same time or within one hour between administration(s) of the anti-CTLA4 binding protein and administration of the one or more additional therapeutic agents). In some embodiments, “in combination with” refers to sequential administration, in which case administration of the anti-CTLA4 binding protein of the invention occurs prior to and/or following, administration of the one or more additional therapeutic agents (e.g., greater than one hour between administration(s) of the anti-CTLA4 binding protein and administration of the one or more additional therapeutic agents). Agents contemplated herein include, but are not limited to, a cytotoxic agent, a cytokine, an agent targeting an immune checkpoint molecule, an agent targeting an immune stimulatory molecule, or a growth inhibitory agent.

The formulation herein may also contain more than one active compound as necessary for the particular indication being treated, preferably those with complementary activities that do not adversely affect each other. Alternatively, or in addition, the composition may comprise a cytotoxic agent, cytokine, agent targeting an immune checkpoint molecule or stimulatory molecule, or growth inhibitory agent. Such molecules are suitably present in combination in amounts that are effective for the purpose intended.

### VI. Articles of Manufacture or Kits

In another aspect, an article of manufacture or kit is provided which comprises an anti-CTLA4 binding protein (e.g., anti-CTLA4 antibody or antigen-binding fragment thereof) and a PD-1 signaling agent (e.g., PD-1 inhibitor or PD-L1 inhibitor) described herein. The article of manufacture or kit may further comprise instructions for use of the binding proteins in the methods of the invention. Thus, in certain embodiments, the article of manufacture or kit comprises instructions for the use of an anti-CTLA4 binding protein (e.g., anti-CTLA4 antibody or antigen-binding fragment thereof) and a PD-1 signaling agent (e.g., PD-1 inhibitor or PD-L1 inhibitor) in methods for treating or preventing a disorder (e.g., a cancer) in an individual comprising administering to the individual an effective amount of an anti-CTLA4 binding protein (e.g., anti-CTLA4 antibody or antigen-binding fragment thereof) and a PD-1 signaling agent (e.g., PD-1 inhibitor or PD-L1 inhibitor). In certain embodiments, the individual is a human. In some embodiments, the individual has a disease selected from the group consisting of include leukemia, lymphoma, head and neck cancer, colorectal cancer, prostate cancer, pancreatic cancer, melanoma, breast cancer, neuroblastoma, lung cancer, ovarian cancer, osteosarcoma, bladder cancer, cervical cancer, liver cancer, kidney cancer, skin cancer or testicular cancer.

The article of manufacture or kit may further comprise a container. Suitable containers include, for example, bottles, vials (e.g., dual chamber vials), syringes (such as single or dual chamber syringes) and test tubes. The container may be formed from a variety of materials such as glass or plastic. The container holds the formulation. In some embodiments, the formulation is a lyophilized formulation.

The article of manufacture or kit may further comprise a label or a package insert, which is on or associated with the container, may indicate directions for reconstitution and/or use of the formulation. The label or package insert may further indicate that the formulation is useful or intended for subcutaneous, intravenous, or other modes of administration for treating or preventing a disorder (e.g., a cancer) in an individual. The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

In a specific embodiment, the present invention provides kits for a single dose-administration unit. Such kits comprise a container of an aqueous formulation of therapeutic antibody, including both single or multi-chambered pre-filled syringes. Exemplary pre-filled syringes are available from Vetter GmbH, Ravensburg, Germany.

The article of manufacture or kit herein optionally further comprises a container comprising a second medicament, wherein the anti-CTLA4 binding protein (e.g., anti-CTLA4 antibody or antigen-binding fragment thereof) is a first medicament, and which article or kit further comprises instructions on the label or package insert for treating the subject with the second medicament (e.g., a PD-1 signaling agent (e.g., PD-1 inhibitor or PD-L1 inhibitor)), in an effective amount.

In another embodiment, provided herein is an article of manufacture or kit comprising the formulations described herein for administration in an auto-injector device. An auto-injector can be described as an injection device that upon activation, will deliver its contents without additional necessary action from the patient or administrator. They are particularly suited for self-medication of therapeutic formulations when the delivery rate must be constant and the time of delivery is greater than a few moments.

### Examples

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Anti-CTLA4 Antibodies in Combination with and PD-1 Antibodies

The present example demonstrates combination therapy of anti-CTLA-4 (e.g., Antibody B) and a PD-1 signaling agent in B-hCTLA4 mice bearing advanced MC38 tumors.

MC38 a murine colon carcinoma cells are maintained under aseptic conditions in Dulbecco's Modified Eagle's medium (DMEM) supplemented with 10% Fetal Bovine Serum (FBS). 0.1 mM nonessential amino acids, 1 mM sodium pyruvate and 10 mM HEPES in a humidified incubator at 37° C. in an atmosphere with 5% CO₂. Upon reaching 50-70% confluence, cells are passaged for a total of three passages, prior to in vivo implantation.

Female B-hCTLA4 mice (13-14-weeks old) are subcutaneously injected with MC38 tumor cells (0.5×10^{f}) in 0.1 mL serum-free medium in the right flank for tumor development. Tumor-bearing animals are randomized study groups when the mean tumor size reach approximately 150 mm³. Groups are Isotype Control (G1: 10 mg/kg), Antibody B monotherapy (G2: 0.3 mg/kg, G3: 1.0 mg/kg, G7: 0.1 mg/kg, G8: 0.03 mg/kg, G9: 0.01 mg/kg), anti-PD-1 monotherapy (G4: 10 mg/kg), Combination therapy with anti-PD-1 (10 mg/kg) with Antibody B (G5: 0.3 mg/kg, G6: 1.0 mg/kg, G10: 0.1 mg/kg, G11: 0.03 mg/kg, G12: 0.01 mg/kg). Isotype control, and Antibody B are given to animals as a single IV injection, whereas anti-PD-1 doses are given Q3D three times by IP injection.

Tumor volume (TV) and body weight (BW) are measured and recorded 2-3 times a week throughout the study. Beyond the dosing phase, animals are monitored until the study endpoint and individual animals are euthanized as each reached TV 2000 mm³ or any other humane endpoint. The end date of each animal is recorded for survival analysis.

Immune activation is measured following combination treatment with anti-CTLA4 Antibody B and anti-PD1 antibody.

| SEQ ID NO | SEQUENCE |
|---|---|
| CDR-L1 Antibody 1 | Gln Ser Leu Leu Asn Ser Asp Gly Asn Thr Tyr |
| 1 | |
| CDR-L2 Antibody 1 | Leu Val Ser |
| 2 | |
| CDR-L3 Antibody 1 | Val Gln Gly Thr His Asp Pro |
| 3 | |
| CDR-H1 Antibody 1 | Tyr His Thr Ile Thr Ser Gly Tyr |
| 4 | |
| CDR-H2 Antibody 1 | Ile Ser Tyr Ser Gly Asn Thr |
| 5 | |
| CDR-H3 Antibody 1 | Ala Ser Met Met Val Pro His Tyr Tyr Val Met Asp Ala |
| 6 | |
| CDR-L1 (Kabat) | Arg Ser Ser Gln Ser Leu Leu Mn Ser Asp Gly Asn Thr Tyr Leu Tyr |
| Antibody 1 | |
| 7 | |
| CDR-L2 (Kabat) | Leu Val Ser Lys Leu Gly Ser |
| Antibody 1 | |
| 8 | |
| CDR-L3 (Kabat) | Val Gln Gly Thr His Asp Pro Trp Thr |
| Antibody 1 | |
| 9 | |
| CDR-H1 (Kabat) | Ser Gly Tyr Asp Trp Thr |
| Antibody 1 | |
| 10 | |
| CDR-H2 (Kabat) | Tyr Ile Ser Tyr Ser Gly Asn Thr Asn Tyr Asn Pro Ser Leu Lys Ser |
| Antibody 1 | |
| 11 | |
| CDR-H3 (Kabat) | Met Met Val Pro His Tyr Tyr Val Met Asp Ala |
| Antibody 1 | |
| 12 | |
| CDR-L1 Antibody 2 | Ser Ala Leu Ser Tyr Met |
| 13 | |
| CDR-L2 Antibody 2 | Gly Thr Ser |
| 14 | |
| CDR-L3 Antibody 2 | His His Trp Ser Asn Thr Gln |
| 15 | |
| CDR-H1 Antibody 2 | Gly Tyr Thr Phe Thr Asn Tyr Phe |
| 16 | |
| CDR-H2 Antibody 2 | Val Asp Pro Glu Gln Gly Arg Ala Asp |
| 17 | |
| CDR-H3 Antibody 2 | Arg Arg Ala Met Asp Asn Tyr Gly Phe Ala Tyr |
| 18 | |
| CDR-L1 (Kabat) | Ser Ala Asn Ser Ala Leu Ser Tyr Met Tyr |
| Antibody 2 | |
| 19 | |
| CDR-L2 (Kabat) | Gly Thr Ser Asn Leu Ala Ser |
| Antibody 2 | |
| 20 | |
| CDR-L3 (Kabat) | His His Trp Ser Asn Thr Gln Trp Thr |
| Antibody 2 | |
| 21 | |
| CDR-H1 (Kabat) | Asn Tyr Phe Met Asn |
| Antibody 2 | |
| 22 | |
| CDR-H2 (Kabat) | Arg Val Asp Pro Glu Gln Gly Arg Ala Asp Tyr Ala Glu Lys Phe Lys |
| Antibody 2 | Lys |
| 23 | |
| CDR-H3 (Kabat) | Arg Ala Met Asp Asn Tyr Gly Phe Ala Tyr |
| Antibody 2 | (Antibody 1) |
| 24 | |
| VL domain | Asp Ile Val Met Thr Gln Thr Pro Leu Ser Leu Ser Val Thr Pro Gly |
| 25 | Gln Pro Ala Ser Ile Ser Cys Arg Ser Ser Gln Ser Leu Leu Asn Ser |
| | Asp Gly Asn Thr Tyr Leu Tyr Trp Tyr Leu Gln Lys Pro Gly Gln Ser |
| | Pro Gln Leu Leu Ile Tyr Leu Val Ser Lys Leu Gly Ser Gly Val Pro |
| | Asn Arg Phe Ser Gly Ser Gly Ser Gly Thr Asp Phe Thr Leu Lys Ile |
| | Ser Arg Val Glu Ala Glu Asp Val Gly Val Tyr Tyr Cys Val Gln Gly |
| | Thr His Asp Pro Trp Thr Phe Gly Gly Gly Thr Lys Val Glu Ile Lys |
| | Arg |
| | (Antibody 1) |
| VH domain | Gln Val Gln Leu Gln Glu Ser Gly Pro Gly Leu Val Lys Pro Ser Glu |
| 26 | Thr Leu Ser Leu Thr Cys Ser Val Thr Tyr His Thr Ile Thr Ser Gly |
| | Tyr Asp Trp Thr Trp Ile Arg Lys Pro Pro Gly Lys Gly Met Glu Trp |
| | Ile Gly Tyr Ile Ser Tyr Ser Gly Asn Thr Asn Tyr Asn Pro Ser Leu |
| | Lys Ser Arg Val Thr Ile Ser Arg Asp Thr Ser Lys Asn Gln Phe Phe |
| | Leu Lys Leu Ser Ser Val Thr Ala Ala Asp Thr Ala Val Tyr Tyr Cys |
| | Ala Ser Met Met Val Pro His Tyr Tyr Val Met Asp Ala Trp Gly Gln |
| | Gly Thr Leu Val Thr Val Ser Ser Ala |
| Light chain | Asp Ile Val Met Thr Gln Thr Pro Leu Ser Leu Ser Val Thr Pro Gly |
| (Antibody 1) | Gln Pro Ala Ser Ile Ser Cys Arg Ser Ser Gln Ser Leu Leu Asn Ser |
| 27 | Asp Gly Asn Thr Tyr Leu Tyr Trp Tyr Leu Gln Lys Pro Gly Gln Ser |
| | Pro Gln Leu Leu Ile Tyr Leu Val Ser Lys Leu Gly Ser Gly Val Pro |
| | Asn Arg Phe Ser Gly Ser Gly Ser Gly Thr Asp Phe Thr Leu Lys Ile |
| | Ser Arg Val Glu Ala Glu Asp Val Gly Val Tyr Tyr Cys Val Gln Gly |
| | Thr His Asp Pro Trp Thr Phe Gly Gly Gly Thr Lys Val Glu lie Lys |
| | Arg Thr Val Ala Ala Pro Ser Val Phe Ile Phe Pro Pro Ser Asp Glu |
| | Gln Leu Lys Ser Gly Thr Ala Ser Val Val Cys Leu Leu Asn Asn Phe |
| | Tyr Pro Arg Glu Ala Lys Val Gln Trp Lys Val Asp Asn Ala Leu Gln |
| | Ser Gly Asn Ser Gln Glu Ser Val Thr Glu Gln Asp Ser Lys Asp Ser |
| | Thr Tyr Ser Leu Ser Ser Thr Leu Thr Leu Ser Lys Ala Asp Tyr Glu |
| | Lys His Lys Val Tyr Ala Cys Glu Val Thr His Gln Gly Leu Ser Ser |
| | Pro Val Thr Lys Ser Phe Asn Arg Gly Glu Cys |
| | (Antibody 1) |
| Heavy chain | Gln Val Gln Leu Gln Glu Ser Gly Pro Gly Leu Val Lys Pro Ser Glu |
| 28 | Thr Leu Ser Leu Thr Cys Ser Val Thr Tyr His Thr lie Thr Ser Gly |
| | Tyr Asp Trp Thr Trp Ile Arg Lys Pro Pro Gly Lys Gly Met Glu Trp |
| | Ile Gly Tyr Ile Ser Tyr Ser Gly Asn Thr Asn Tyr Asn Pro Ser Leu |
| | Lys Ser Arg Val Thr Ile Ser Arg Asp Thr Ser Lys Asn Gln Phe Phe |
| | Leu Lys Leu Ser Set Val Thr Ala Ala Asp Thr Ala Val Tyr Tyr Cys |
| | Ala Ser Met Met Val Pro His Tyr Tyr Val Met Asp Ala Trp Gly Gln |
| | Gly Thr Leu Val Thr Val Ser Ser Ala Ser Thr Lys Gly Pro Ser Val |
| | Phe Pro Leu Ala Pro Ser Ser Lys Ser Thr Ser Gly Gly Thr Ala Ala |
| | Leu Gly Cys Leu Val Lys Asp Tyr Phe Pro Glu Pro Val Thr Val Ser |
| | Trp Asn Ser Gly Ala Leu Thr Ser Gly Val His Thr Phe Pro Ala Val |
| | Leu Gln Ser Ser Gly Leu Tyr Ser Leu Ser Ser Val Val Thr Val Pro |
| | Ser Ser Ser Leu Gly Thr Gln Thr Tyr lie Cys Asn Val Asn His Lys |
| | Pro Ser Asn Thr Lys Val Asp Lys Lys Val Glu Pro Lys So. Cys Asp |
| | Lys Thr His Thr Cys Pro Pro Cys Pro Ala Pro Glu Leu Leu Gly Gly |
| | Pro Ser Val Phe Leu Phe Pro Pro Lys Pro Lys Asp Thr Leu Met Ile |
| | Ser Arg Thr Pro Glu Val Thr Cys Val Val Val Asp Val Ser His Glu |
| | Asp Pro Glu Val Lys Phe Asn Trp Tyr Val Asp Gly Val Glu Val His |
| | Asn Ala Lys Thr Lys Pro Arg Glu Glu Gln Tyr Asn Ser Thr Tyr Arg |
| | Val Val Ser Val Leu Thr Val Leu His Gln Asp Trp Leu Asn Gly Lys |
| | Glu Tyr Lys Cys Lys Val Ser Asn Lys Ala Leu Pro Ala Pro Ile Glu |
| | Lys Thr Ile Ser Lys Ala Lys Gly Gln Pro Arg Glu Pro Gln Val Tyr |
| | Thr Leu Pro Pro Ser Arg Asp Glu Leu Thr Lys Asn Gln Val Ser Leu |
| | Thr Cys Len Val Lys Gly Phe Tyr Pro Ser Asp Ile Ala Val Glu Trp |
| | Glu Ser Asn Gly Gln Pro Glu Asn Asn Tyr Lys Thr Thr Pro Pro Val |
| | Leu Asp Ser Asp Gly Ser Phe Phe Leu Tyr Ser Lys Leu Thr Val Asp |
| | Lys Ser Arg Trp Gln Gln Gly Asn Val Phe Ser Cys Ser Val Met His |
| | Glu Ala Leu His Asn His Tyr Thr Gln Lys Ser Leu Ser Leu Ser Pro |
| | Gly Lys |
| Heavy chain | (Antibody 1-S239D, 1332E) |
| 29 | Gln Val Gln Leu Gln Glu Ser Gly Pro Gly Leu Val Lys Pro Ser Glu |
| | Thr Leu Ser Leu Thr Cys Ser Val Thr Tyr His Thr lie Thr Ser Gly |
| | Tyr Asp Trp Thr Trp Ile Arg Lys Pro Pro Gly Lys Gly Met Glu Trp |
| | Ile Gly Tyr Ile Ser Tyr Ser Gly Asn Thr Asn Tyr Asn Pro Ser Leu |
| | Lys Ser Arg Val Thr Ile Ser Arg Asp Thr Ser Lys Asn Gln Phe Phe |
| | Leu Lys Leu Ser Ser Val Thr Ala Ala Asp Thr Ala Val Tyr Tyr Cys |
| | Ala Ser Met Met Val Pro His Tyr Tyr Val Met Asp Ala Trp Gly Gln |
| | Gly Thr Len Val Thr Val Ser Ser Ala Ser Thr Lys Gly Pro Ser Val |
| | Phe Pro Leu Ala Pro Ser Ser Lys Ser Thr Ser Gly Gly Thr Ala Ala |
| | Leu Gly Cys Leu Val Lys Asp Tyr Phe Pro Glu Pro Val Thr Val Ser |
| | Trp Asn Ser Gly Ala Leu Thr Ser Gly Val His Thr Phe Pro Ala Val |
| | Leu Gln Ser Ser Gly Leu Tyr Ser Leu Ser Ser Val Val Thr Val Pro |
| | Ser Ser Ser Leu Gly Thr Gln Thr Tyr Ile Cys Asn Val Asn His Lys |
| | Pro Ser Asn Thr Lys Val Asp Lys Lys Val Glu Pro Lys Ser Cys Asp |
| | Lys Thr His Thr Cys Pro Pro Cys Pro Ala Pro Glu Leu Leu Gly Gly |
| | Pro Asp Val Phe Leu Phe Pro Pro Lys Pro Lys Asp Thr Leu Met Ile |
| | Ser Arg Thr Pro Glu Val Thr Cys Val Val Val Asp Val Ser His Glu |
| | Asp Pro Glu Val Lys Phe Asn Trp Tyr Val Asp Gly Val Glu Val His |
| | Asn Ala Lys Thr Lys Pro Arg Glu Glu Gln Tyr Asn Ser Thr Tyr Arg |
| | Val Val Ser Val Leu Thr Val Leu His Gln Asp Trp Leu Asn Gly Lys |
| | Glu Tyr Lys Cys Lys Val Ser Asn Lys Ala Leu Pro Ala Pro Glu Glu |
| | Lys Thr Ile Ser Lys Ala Lys Gly Gln Pro Arg Glu Pro Gln Val Tyr |
| | Thr Leu Pro Pro Ser Arg Asp Glu Leu Thr Lys Asn Gln Val Ser Leu |
| | Thr Cys Leu Val Lys Gly Phe Tyr Pro Ser Asp Ile Ala Val Glu Trp |
| | Glu Ser Asn Gly Gln Pro Glu Asn Asn Tyr Lys Thr Thr Pro Pro Val |
| | Leu Asp Ser Asp Gly Ser Phe Phe Leu Tyr Ser Lys Leu Thr Val Asp |
| | Lys Ser Arg Trp Gln Gln Gly Asn Val Phe Ser Cys Ser Val Met His |
| | Glu Ala Leu His Asn His Tyr Thr Gln Lys Ser Leu Ser Leu Ser Pro |
| | Gly Lys |
| | (Antibody 2) |
| VL domain | Glu Ile Val Leu Thr Gln Ser Pro Asp Phe Gln Ser Val Thr Pro Lys |
| 30 | Glu Lys Val Thr Ile Thr Cys Ser Ala Asn Ser Ala Leu Ser Tyr Met |
| | Tyr Trp Tyr Gln Gln Lys Pro Asp Gln Ser Pro Lys Leu Trp Val His |
| | Gly Thr Ser Asn Leu Ala Ser Gly Val Pro Ser Arg Phe Ser Gly Ser |
| | Gly Ser Gly Thr Asp Phe Thr Leu Thr Ile Asn Ser Leu Glu Ala Glu |
| | Asp Ala Ala Thr Tyr Tyr Cys His His Trp Ser Asn Thr Gln Trp Thr |
| | Phe Gly Gly Gly Thr Lys Val Glu Ile Lys Arg |
| | (Antibody 2) |
| VH domain | Gln Val Gln Leu Val Gln Ser Gly Ala Glu Val Lys Lys Pro Gly Ser |
| 31 | Ser Val Lys Val Ser Cys Lys Ala Ser Gly Tyr Thr Phe Thr Asn Tyr |
| | Phe Met Asn Trp Val Arg Gln Ala Pro Gly Gln Gly Leu Glu Trp Met |
| | Gly Arg Val Asp Pro Glu Gln Gly Arg Ala Asp Tyr Ala Glu Lys Phe |
| | Lys Lys Arg Val Thr Ile Thr Ala Asp Lys Ser Thr Ser Thr Ala Tyr |
| | Met Glu Leu Ser Ser Leu Arg Ser Glu Asp Thr Ala Val Tyr Tyr Cys |
| | Ala Arg Arg Ala Met Asp Asn Tyr Gly Phe Ala Tyr Trp Gly Gln Gly |
| | Thr Leu Val Thr Val Ser Ser Ala |
| | (Antibody 2) |
| Light chain | Glu Ile Val Leu Thr Gln Ser Pro Asp Phe Gln Ser Val Thr Pro Lys |
| 32 | Glu Lys Val Thr Ile Thr Cys Ser Ala Asn Ser Ala Leu Ser Tyr Met |
| | Tyr Trp Tyr Gln Gln Lys Pro Asp Gln Ser Pro Lys Leu Trp Val His |
| | Gly Thr Ser Asn Leu Ala Ser Gly Val Pro Ser Arg Phe Ser Gly Ser |
| | Gly Ser Gly Thr Asp Phe Thr Leu Thr Ile Asn Ser Leu Glu Ala Glu |
| | Asp Ala Ala Thr Tyr Tyr Cys His His Trp Ser Asn Thr Gln Trp Thr |
| | Phe Gly Gly Gly Thr Lys Val Glu Ile Lys Arg Thr Val Ala Ala Pro |
| | Ser Val Phe Ile Phe Pro Pro Ser Asp Glu Gln Leu Lys Ser Gly Thr |
| | Ala Ser Val Val Cys Leu Len Asn Asn Phe Tyr Pro Arg Glu Ala Lys |
| | Val Gln Trp Lys Val Asp Asn Ala Leu Gln Ser Gly Asn Ser Gln Glu |
| | Ser Val Thr Glu Gln Asp Ser Lys Asp Ser Thr Tyr Ser Leu Ser Ser |
| | Thr Leu Thr Leu Ser Lys Ala Asp Tyr Glu Lys His Lys Val Tyr Ala |
| | Cys Glu Val Thr His Gln Gly Leu Ser Ser Pro Val Thr Lys Ser Phe |
| | Asn Arg Gly Glu Cys |
| | (Antibody 2) |
| Heavy chain | Gln Val Gln Leu Val Gln Ser Gly Ala Glu Val Lys Lys Pro Gly Ser |
| 33 | Ser Val Lys Val Ser Cys Lys Ala Ser Gly Tyr Thr Phe Thr Asn Tyr |
| | Phe Met Asn Trp Val Arg Gln Ala Pro Gly Gln Gly Leu Glu Trp Met |
| | Gly Arg Val Asp Pro Glu Gln Gly Arg Ala Asp Tyr Ala Glu Lys Phe |
| | Lys Lys Arg Val Thr Ile Thr Ala Asp Lys Ser Thr Ser Thr Ala Tyr |
| | Met Glu Leu Ser Ser Leu Arg Ser Glu Asp Thr Ala Val Tyr Tyr Cys |
| | Ala Arg Arg Ala Met Asp Asn Tyr Gly Phe Ala Tyr Trp Gly Gln Gly |
| | Thr Leu Val Thr Val Ser Ser Ala Ser Thr Lys Gly Pro Ser Val Phe |
| | Pro Leu Ala Pro Ser Ser Lys Ser Thr Ser Gly Gly Thr Ala Ala Leu |
| | Gly Cys Leu Val Lys Asp Tyr Phe Pro Glu Pro Val Thr Val Ser Trp |
| | Asn Ser Gly Ala Leu Thr Ser Gly Val His Thr Phe Pro Ala Val Leu |
| | Gln Ser Ser Gly Leu Tyr Ser Leu Ser Ser Val Val Thr Val Pro Ser |
| | Ser Ser Leu Gly Thr Gln Thr Tyr Ile Cys Asn Val Asn his Lys Pro |
| | Ser Asn Thr Lys Val Asp Lys Lys Val Glu Pro Lys Ser Cys Asp Lys |
| | Thr His Thr Cys Pro Pro Cys Pro Ala Pro Glu Leu Leu Gly Gly Pro |
| | Ser Val Phe Leu Phe Pro Pro Lys Pro Lys Asp Thr Leu Met Ile Ser |
| | Arg Thr Pro Glu Val Thr Cys Val Val Val Asp Val Ser His Glu Asp |
| | Pro Glu Val Lys Phe Asn Trp Tyr Val Asp Gly Val Glu Val His Asn |
| | Ala Lys Thr Lys Pro Arg Glu Glu Gln Tyr Asn Ser Thr Tyr Arg Val |
| | Val Ser Val Leu Thr Val Leu His Gln Asp Trp Leu Asn Gly Lys Glu |
| | Tyr Lys Cys Lys Val Ser Asn Lys Ala Leu Pro Ala Pro Ile Glu Lys |
| | Thr Ile Ser Lys Ala Lys Gly Gln Pro Arg Glu Pro Gln Val Tyr Thr |
| | Leu Pro Pro Ser Arg Asp Glu Leu Thr Lys Asn Gln Val Ser Leu Thr |
| | Cys Leu Val Lys Gly Phe Tyr Pro Ser Asp Ile Ala Val Glu Trp Glu |
| | Ser Asn Gly Gln Pro Glu Asn Asn Tyr Lys Thr Thr Pro Pro Val Leu |
| | Asp Ser Asp Gly Ser Phe Phe Leu Tyr Ser Lys Leu Thr Val Asp Lys |
| | Ser Arg Trp Gln Gln Gly Asn Val Phe Ser Cys Ser Val Met His Glu |
| | Ala Leu His Asn His Tyr Thr Gln Lys Ser Leu Ser Leu Ser Pro Gly |
| | Lys |
| | (Antibody 2-S239D, 1332E) |
| Heavy chain | Gln Val Gln Leu Val Gln Ser Gly Ala Glu Val Lys Lys Pro Gly Ser |
| 34 | Ser Val Lys Val Ser Cys Lys Ala Ser Gly Tyr Thr Phe Thr Asn Tyr |
| | Phe Met Asn Trp Val Arg Gln Ala Pro Gly Gln Gly Leu Glu Trp Met |
| | Gly Arg Val Asp Pro Glu Gln Gly Arg Ala Asp Tyr Ala Glu Lys Phe |
| | Lys Lys Arg Val Thr Ile Thr Ala Asp Lys Ser Thr Ser Thr Ala Tyr |
| | Met Glu Leu Ser Ser Leu Arg Ser Glu Asp Thr Ala Val Tyr Tyr Cys |
| | Ala Arg Arg Ala Met Asp Asn Tyr Gly Phe Ala Tyr Trp Gly Gln Gly |
| | Thr Leu Val Thr Val Ser Ser Ala Ser Thr Lys Gly Pro Ser Val Phe |
| | Pro Leu Ala Pro Ser Ser Lys Ser Thr Ser Gly Gly Thr Ala Ala Leu |
| | Gly Cys Leu Val Lys Asp Tyr Phe Pro Glu Pro Val Thr Val Ser Trp |
| | Asn Ser Gly Ala Leu Thr Ser Gly Val His Thr Phe Pro Ala Val Leu |
| | Gln Ser Ser Gly Leu Tyr Ser Leu Ser Ser Val Val Thr Val Pro Ser |
| | Ser Ser Leu Gly Thr Gln Thr Tyr Ile Cys Asn Val Asn His Lys Pro |
| | Ser Asn Thr Lys Val Asp Lys Lys Val Glu Pro Lys Ser Cys Asp Lys |
| | Thr His Thr Cys Pro Pro Cys Pro Ala Pro Glu Leu Leu Gly Gly Pro |
| | Asp Val Phe Leu Phe Pro Pro Lys Pro Lys Asp Thr Leu Met Ile Ser |
| | Arg Thr Pro Glu Val Thr Cys Val Val Val Asp Val Ser His Glu Asp |
| | Pro Glu Val Lys Phe Asn Trp Tyr Val Asp Gly Val Glu Val His Asn |
| | Ala Lys Thr Lys Pro Arg Glu Glu Gln Tyr Asn Ser Thr Tyr Arg Val |
| | Val Ser Val Leu Thr Val Leu His Gln Asp Trp Leu Asn Gly Lys Glu |
| | Tyr Lys Cys Lys Val Ser Asn Lys Ala Leu Pro Ala Pro Glu Glu Lys |
| | Thr Ile Ser Lys Ala Lys Gly Gln Pro Arg Glu Pro Gln Val Tyr Thr |
| | Leu Pro Pro Ser Arg Asp Glu Leu Thr Lys Asn Gln Val Ser Leu Thr |
| | Cys Leu Val Lys Gly Phe Tyr Pro Ser Asp Ile Ala Val Glu Trp Glu |
| | Ser Asn Gly Gln Pro Glu Asn Asn Tyr Lys Thr Thr Pro Pro Val Leu |
| | Asp Ser Asp Gly Ser Phe Phe Leu Tyr Ser Lys Leu Thr Val Asp Lys |
| | Ser Arg Trp Gln Gln Gly Asn Val Phe Ser Cys Ser Val Met His Glu |
| | Ala Leu His Asn His Tyr Thr Gln Lys Ser Leu Ser Leu Ser Pro Gly |
| | Lys |
| Heavy Chain | Ser Thr Lys Gly Pro Ser Val Phe Pro Leu Ala Pro Ser Ser Lys Ser |
| Constant Domain | Thr Ser Gly Gly Thr Ala Ala Leu Gly Cys Leu Val Lys Asp Tyr Phe |
| 35 | Pro Glu Pro Val Thr Val Ser Trp Asn Ser Gly Ala Leu Thr Ser Gly |
| | Val His Thr Phe Pro Ala Val Leu Gln Ser Ser Gly Leu Tyr Ser Leu |
| | Ser Ser Val Val Thr Val Pro Ser Ser Ser Leu Gly Thr Gln Thr Tyr |
| | Ile Cys Asn Val Asn His Lys Pro Ser Asn Thr Lys Val Asp Lys Lys |
| | Val Glu Pro Lys Ser Cys Asp Lys Thr His Thr Cys Pro Pro Cys Pro |
| | Ala Pro Glu Leu Leu Gly Gly Pro Ser Val Phe Leu Phe Pro Pro Lys |
| | Pro Lys Asp Thr Leu Met Ile Ser Arg Thr Pro Glu Val Thr Cys Val |
| | Val Val Asp Val Ser His Glu Asp Pro Glu Val Lys Phe Asn Trp Tyr |
| | Val Asp Gly Val Glu Val His Asn Ala Lys Thr Lys Pro Arg Glu Glu |
| | Gln Tyr Asn Ser Thr Tyr Arg Val Val Ser Val Leu Thr Val Leu His |
| | Gln Asp Trp Leu Asn Gly Lys Glu Tyr Lys Cys Lys Val Ser Asn Lys |
| | Ala Leu Pro Ala Pro Ile Glu Lys Thr Ile Ser Lys Ala Lys Gly Gln |
| | Pro Arg Glu Pro Gln Val Tyr Thr Leu Pro Pro Ser Arg Asp Glu Leu |
| | Thr Lys Asn Gln Val Ser Leu Thr Cys Leu Val Lys Gly Phe Tyr Pro |
| | Ser Asp Ile Ala Val Glu Trp Glu Ser Asn Gly Gln Pro Glu Asn Asn |
| | Tyr Lys Thr Thr Pro Pro Val Leu Asp Ser Asp Gly Ser Phe Pk Leu |
| | Tyr Ser Lys Leu Thr Val Asp Lys Ser Arg Trp Gln Gln Gly Asn Val |
| | Phe Ser Cys Ser Val Met His Glu Ala Leu His Asn His Tyr Thr Gln |
| | Lys Ser Leu Ser Leu Ser Pro Gly Lys |
| Heavy Chain | Ser Thr Lys Gly Pro Ser Val Phe Pro Leu Ala Pro Ser Ser Lys Ser |
| Constant Domain | Thr Ser Gly Gly Thr Ala Ala Leu Gly Cys Leu Val Lys Asp Tyr Phe |
| S239 | Pro Glu Pro Val Thr Val Ser Trp Asn Ser Gly Ala Leu Thr Ser Gly |
| 36 | Val His Thr Phe Pro Ala Val Leu Gln Ser Ser Gly Leu Tyr Ser Leu |
| | Ser Ser Val Val Thr Val Pro Ser Ser Ser Leu Gly Thr Gln Thr Tyr |
| | Ile Cys Asn Val Asn His Lys Pro Ser Asn Thr Lys Val Asp Lys Lys |
| | Val Glu Pro Lys Ser Cys Asp Lys Thr His Thr Cys Pro Pro Cys Pro |
| | Ala Pro Glu Leu Leu Gly Gly Pro Asp Val Phe Leu Phe Pro Pro Lys |
| | Pro Lys Asp Thr Leu Met Ile Ser Arg Thr Pro Glu Val Thr Cys Val |
| | Val Val Asp Val Ser His Glu Asp Pro Glu Val Lys Phe Asn Trp Tyr |
| | Val Asp Gly Val Glu Val His Asn Ala Lys Thr Lys Pro Arg Glu Glu |
| | Gln Tyr Asn Ser Thr Tyr Arg Val Val Ser Val Leu Thr Val Leu His |
| | Gln Asp Trp Leu Asn Gly Lys Glu Tyr Lys Cys Lys Val Ser Asn Lys |
| | Ala Leu Pro Ala Pro Ile Glu Lys Thr Ile Ser Lys Ala Lys Gly Gln |
| | Pro Arg Glu Pro Gln Val Tyr Thr Leu Pro Pro Ser Arg Asp Glu Leu |
| | Thr Lys Asn Gln Val Ser Leu Thr Cys Leu Val Lys Gly Phe Tyr Pro |
| | Ser Asp Ile Ala Val Glu Trp Glu Ser Asn Gly Gln Pro Glu Asn Asn |
| | Tyr Lys Thr Thr Pro Pro Val Leu Asp Ser Asp Gly Ser Phe Phe Leu |
| | Tyr Ser Lys Leu Thr Val Asp Lys Ser Arg Trp Gln Gln Gly Asn Val |
| | Phe Ser Cys Ser Val Met His Glu Ala Leu His Asn His Tyr Thr Gln |
| | Lys Ser Leu Ser Leu Ser Pro Gly Lys |
| | I332E |
| Heavy Chain | Ser Thr Lys Gly Pro Ser Val Phe Pro Leu Ala Pro Ser Ser Lys Ser |
| Constant Domain | Thr Ser Gly Gly Thr Ala Ala Leu Gly Cys Leu Val Lys Asp Tyr Phe |
| 37 | Pro Glu Pro Val Thr Val Ser Trp Asn Ser Gly Ala Leu Thr Ser Gly |
| | Val His Thr Phe Pro Ala Val Leu Gln Ser Ser Gly Leu Tyr Ser Leu |
| | Ser Ser Val Val Thr Val Pro Ser Ser Ser Leu Gly Thr Gln Thr Tyr |
| | Ile Cys Asn Val Asn His Lys Pro Ser Asn Thr Lys Val Asp Lys Lys |
| | Val Glu Pro Lys Ser Cys Asp Lys Thr His Thr Cys Pro Pro Cys Pro |
| | Ala Pro Glu Leu Leu Gly Gly Pro Ser Val Phe Leu Phe Pro Pro Lys |
| | Pro Lys Asp Thr Leu Met Ile Ser Arg Thr Pro Glu Val Thr Cys Val |
| | Val Val Asp Val Ser His Glu Asp Pro Glu Val Lys Phe Asn Trp Tyr |
| | Val Asp Gly Val Glu Val His Asn Ala Lys Thr Lys Pro Arg Glu Glu |
| | Gln Tyr Asn Ser Thr Tyr Arg Val Val Ser Val Leu Thr Val Leu His |
| | Gln Asp Trp Leu Asn Gly Lys Glu Tyr Lys Cys Lys Val Ser Asn Lys |
| | Ala Leu Pro Ala Pro Glu Glu Lys Thr Ile Ser Lys Ala Lys Gly Gln |
| | Pro Arg Glu Pro Gln Val Tyr Thr Leu Pro Pro Ser Arg Asp Glu Leu |
| | Thr Lys Asn Gln Val Ser Leu Thr Cys Leu Val Lys Gly Phe Tyr Pro |
| | Ser Asp lie Ala Val Glu Trp Glu Ser Asn Gly Gln Pro Glu Asn Asn |
| | Tyr Lys Thr Thr Pro Pro Val Leu Asp Ser Asp Gly Ser Phe Phe Leu |
| | Tyr Ser Lys Leu Thr Val Asp Lys Ser Arg Trp Gln Gln Gly Asn Val |
| | Phe Ser Cys Ser Val Met His Glu Ala Leu His Asn His Tyr Thr Gln |
| | Lys Ser Leu Ser Leu Ser Pro Gly Lys |
| | S239D, I332E |
| HEavy Chain | Ser Thr Lys Gly Pro Ser Val Phe Pro Leu Ala Pro Ser Ser Lys Ser |
| Constant Domain | Thr Ser Gly Gly Thr Ala Ala Leu Gly Cys Leu Val Lys Asp Tyr Phe |
| 38 | Pro Glu Pro Val Thr Val Ser Trp Asn Ser Gly Ala Leu Thr Ser Gly |
| | Val His Thr Phe Pro Ala Val Leu Gln Ser Ser Gly Leu Tyr Ser Leu |
| | Ser Ser Val Val Thr Val Pro Ser Ser Ser Leu Gly Thr Gln Thr Tyr |
| | Ile Cys Asn Val Asn His Lys Pro Ser Asn Thr Lys Val Asp Lys Lys |
| | Val Glu Pro Lys Ser Cys Asp Lys Thr His Thr Cys Pro Pro Cys Pro |
| | Ala Pro Glu Leu Leu Gly Gly Pro Asp Val Phe Leu Phe Pro Pro Lys |
| | Pro Lys Asp Thr Leu Met Ile Ser Arg Thr Pro Glu Val Thr Cys Val |
| | Val Val Asp Val Ser His Glu Asp Pro Glu Val Lys Phe Asn Trp Tyr |
| | Val Asp Gly Val Glu Val His Asn Ala Lys Thr Lys Pro Arg Glu Glu |
| | Gln Tyr Asn Ser Thr Tyr Arg Val Val Ser Val Leu Thr Val Leu His |
| | Gln Asp Trp Leu Asn Gly Lys Glu Tyr Lys Cys Lys Val Ser Asn Lys |
| | Ala Leu Pro Ala Pro Glu Glu Lys Thr Ile Ser Lys Ala Lys Gly Gln |
| | Pro Arg Glu Pro Gln Val Tyr Thr Leu Pro Pro Ser Arg Asp Glu Leu |
| | Thr Lys Asn Gln Val Ser Leu Thr Cys Leu Val Lys Gly Phe Tyr Pro |
| | Ser Asp lie Ala Val Glu Trp Glu Ser Asn Gly Gln Pro Glu Asn Asn |
| | Tyr Lys Thr Thr Pro Pro Val Leu Asp Ser Asp Gly Ser Phe Phe Leu |
| | Tyr Ser Lys Leu Thr Val Asp Lys Ser Arg Trp Gln Gln Gly Asn Val |
| | Phe Ser Cys Ser Val Met His Glu Ala Leu His Asn His Tyr Thr Gln |
| | Lys Ser Leu Ser Leu Ser Pro Gly Lys |
| Light chain | Thr Val Ala Ala Pro Ser Val Phe Ile Phe Pro Pro Ser Asp Glu Gln |
| Constant Domain | Leu Lys Ser Gly Thr Ala Ser Val Val Cys Leu Leu Asn Asn Phe Tyr |
| 39 | Pro Arg Glu Ala Lys Val Gln Trp Lys Val Asp Asn Ala Leu Gln Ser |
| | Gly Asn Ser Gln Glu Ser Val Thr Glu Gln Asp Ser Lys Asp Ser Thr |
| | Tyr Ser Leu Ser Ser Thr Leu Thr Leu Ser Lys Ala Asp Tyr Glu Lys |
| | His Lys Val Tyr Ala Cys Glu Val Thr His Gln Gly Leu Ser Ser Pro |
| | Val Thr Lys Ser Phe Asn Arg Gly Glu Cys |

1. A method of treating cancer in a subject, the method comprising administering to the subject:
(a) an anti-CTLA4 antibody comprising a heavy chain variable region (vH) CDR1 comprising NYFMN (SEQ ID NO: 22), a vH CDR2 comprising RVDPEQGRADYAEKFKK (SEQ ID NO: 23), a vH CDR3 comprising RAMDNYGFAY (SEO ID NO: 24); and
a light chain variable region (vL) CDR1 comprising SANSALSYMY (SEO ID NO: 19), a vL CDR2 comprising GTSNLAS (SEQ ID NO: 20), a vL CDR3 comprising HHWSNTQWT (SEQ ID NO: 21);
(b) a PD-1 or PD-L1 inhibitor.

2. The method of claim 1, wherein the anti-CTLA4 antibody is administered at an effective dose of between about 0.1-20 mg/kg.

3. (canceled)

4. The method of claim 1, wherein the anti-CTLA4 antibody comprises a heavy chain constant domain comprising amino acid substitutions S239D or I332E or both, wherein the amino acid residues are numbered according to the EU index as in Kabat.

5. The method of claim 1, wherein the anti-CTLA4 antibody comprises a vH that is at least 90% identical to SEQ ID NO: 31.

6. The method of claim 1, wherein the anti-CTLA4 antibody comprises a vL that is at least 90% identical to SEQ ID NO: 30.

7.-8. (canceled)

9. The method of claim 1, wherein the anti-CTLA4 antibody or antigen-binding fragment thereof is conjugated to an agent, wherein the agent is an inhibitor of tubulin polymerization, a DNA damaging agent, or a DNA synthesis inhibitor.

10. The method of claim 1, wherein the agent is a maytansinoid, an auristatin, a pyrrolobenzodiazepine (PBD) dimer, a calicheamicin, a duocarmycin, an indo-linobenzodiazepine dimer, or exatecan derivative Dxd.

11. The method claim 1, wherein the PD-1/PD-L1 inhibitor is an antibody.

12. The method of claim 1, wherein the PD-1/PD-L1 inhibitor is a PD-1 antibody.

13. The method of claim 12, wherein the anti-PD-1 antibody is selected from nivolumab, pembrolizumab, and cemiplimab.

14. The method of claim 12, wherein the effective dose of the PD-1 antibody is between 1-10 mg/kg.

15.-18. (canceled)

19. The method of claim 12, wherein the anti-PD-1 antibody is administered weekly, every other week, every 3 weeks, every 4 weeks, every 5 weeks, every 6 weeks, or monthly.

20. The method of claim 1, wherein the PD-1/PD-L1 inhibitor is a PD-L1 antibody.

21. The method of claim 20, wherein the anti-PD-L1 antibody is selected from atezolizumab, avelumab, durvalumab.

22. The method of claim 21, wherein the anti-PD-L1 antibody is administered at an effective dose of between 200-2000 mg.

23. The method of claim 20, wherein the anti-PD-L1 antibody is administered weekly, every other week, every 3 weeks, every 4, weeks, every 6 weeks or monthly.

24. The method of claim 1, wherein the anti-PD-1 and anti-CTLA4 antibodies are formulated for intravenous administration.

25. The method of claim 1, wherein the anti-PD-1 and anti-CTLA4 antibodies are formulated together in the same composition.

26. The method of claim 1, wherein the anti-PD-1 and anti-CTLA4 antibodies are formulated separately.

27. The method of claim 1, wherein the anti-PD-1 antibody is administered concurrently with the anti-CTLA4 antibody.

28. The method of claim 1, wherein the cancer is leukemia, lymphoma, head and neck cancer, colorectal cancer, prostate cancer, pancreatic cancer, melanoma, myeloma, breast cancer, neuroblastoma, lung cancer, ovarian cancer, osteosarcoma, bladder cancer, cervical cancer, liver cancer, kidney cancer, skin cancer, testicular cancer, or cutaneous squamous cell carcinoma (CSCC).

29.-31. (canceled)
